SEQUENCE LISTING (23 sequences)

SEQ ID NO: 1 - protein, 920 aa, Artificial Sequence
  Synthetic polypeptide
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 2 - protein, 644 aa, Artificial Sequence
  Synthetic polypeptide
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGEKFRVGNCKHLKMTRP

SEQ ID NO: 3 - protein, 9 aa, Artificial Sequence
  Synthetic polypeptide
YPYDVPDYA

SEQ ID NO: 4 - protein, 943 aa, Artificial Sequence
  Synthetic polypeptide
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEEIPEERDSGNSLSGLSTLVFVLPGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 5 - protein, 640 aa, Artificial Sequence
  Synthetic polypeptide
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKL

SEQ ID NO: 6 - protein, 648 aa, Artificial Sequence
  Synthetic polypeptide
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGAAVVVSERILRVFGVSEWLP

SEQ ID NO: 7 - protein, 687 aa, Artificial Sequence
  Synthetic polypeptide
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGGKQKYLCASRNDCTIDKFRRKNCPSCRLRKCYEAGMTLGAAVVVSERILRVFGVSEWLP

SEQ ID NO: 8 - protein, 701 aa, Artificial Sequence
  Synthetic polypeptide
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGFFRMNKLKESSDTNPKPYCMAAPMGLTENNRNRKKSYRETNLKAVSWPLNHTGKQKYLCASRNDCTIDKFRRKNCPSCRLRKCYEAGMTLGAAVVVSERILRVFGVSEWLP

SEQ ID NO: 9 - protein, 740 aa, Artificial Sequence
  Synthetic polypeptide
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGGFFRMNKLKESSDTNPKPYCMAAPMGLTENNRNRKKSYRETNLKAVSWPLNHTGKQKYLCASRNDCTIDKFRRKNCPSCRLRKCYEAGMTLGAAVVVSERILRVFGVSEWLP

SEQ ID NO: 10 - protein, 629 aa, Artificial Sequence
  Synthetic polypeptide
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGD

SEQ ID NO: 11 - protein, 634 aa, Artificial Sequence
  Synthetic polypeptide
MEVQLGLGRVYPRPPSKTYRGAFQNLFQSVREVIQNPGPRHPEAASAAPPGASLLLLEASGCHYGALTCGSCKVFFKRAAEGKQKYLCASRNDCTIDKFRRKNCPSCRLRKCYEAGMTLGAGSRVSQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGD

SEQ ID NO: 12 - protein, 638 aa, Artificial Sequence
  Synthetic polypeptide
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGGFDNLCELSS

SEQ ID NO: 13 - protein, 651 aa, Artificial Sequence
  Synthetic polypeptide
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGDNLPEQAAFWRHLHIFWDHVVKK

SEQ ID NO: 14 - protein, 665 aa, Artificial Sequence
  Synthetic polypeptide
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTPSSGTNSVFLPHRDVVRTGCRSNSGYHSCSCEYHDYCFL

SEQ ID NO: 15 - protein, 648 aa, Artificial Sequence
  Synthetic polypeptide
MEVQLGLGRVYPRPPSKTYRGAFQNLFQSVREVIQNPGPRHPEAASAAPPGASLLLLPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGGKILFFLFLLLPLSPFSLIFQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLEL

SEQ ID NO: 16 - protein, 734 aa, Artificial Sequence
  Synthetic polypeptide
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPDCERAASVHF

SEQ ID NO: 17 - protein, 819 aa, Artificial Sequence
  Synthetic polypeptide
MEVQLGLGRVYPRPPSKTYRGAFQNLFQSVREVIQNPGPRHPEAASAAPPGASLLLLEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSINHTQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGD

SEQ ID NO: 18 - protein, 877 aa, Artificial Sequence
  Synthetic polypeptide
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPITPDAMYL

SEQ ID NO: 19 - protein, 851 aa, Artificial Sequence
  Synthetic polypeptide
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSISPVKEQRDKKSRGHDTLYFTSSRQMNVESIKTQWN

SEQ ID NO: 20 - protein, 895 aa, Artificial Sequence
  Synthetic polypeptide
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMITPDAMYL

SEQ ID NO: 21 - protein, 846 aa, Artificial Sequence
  Synthetic polypeptide
MEVQLGLGRVYPRPPSKTYRGAFQNLFQSVREVIQNPGPRHPEAASAAPPGASLLLLPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSISSRSHLMPCTCERGCSFVLEALSEQTRHLDQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLEL

SEQ ID NO: 22 - protein, 572 aa, Artificial Sequence
  Synthetic polypeptide
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRNTRRKRLWKLIIRSINSCICSPRETEVPVRQQK

SEQ ID NO: 23 - protein, 388 aa, Artificial Sequence
  Synthetic polypeptide
MILWLHSLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

(19) United States
(12) Patent Application Publication
(10) Pub. No.: US 2019/0060296 A1
(43) Pub. Date: Feb. 28, 2019

(54) DISCOVERY OF SMALL MOLECULES THAT TARGET THE ANDROGEN RECEPTOR AND USES

(71) Applicant: Massachusetts Institute of Technology, Cambridge, MA (US)

(72) Inventors: Angela N. Koehler, Belmont, MA (US); Marius S. Pop, Waltham, MA (US)

(21) Appl. No.: 16/048,088

(22) Filed: Jul. 27, 2018

Related U.S. Application Data
(60) Provisional application No. 62/538,415, filed on Jul. 28, 2017.

(51) Int. Cl. A61K 31/445; A61K 31/519; A61K 31/5513; A61K 31/506

(52) U.S. Cl. CPC A61K 31/445; A61K 31/506; A61K 31/5513; A61K 31/519

(57) ABSTRACT

Provided herein are methods useful for identifying compounds that modulate the activity of an androgen receptor, or variant thereof. In particular, this invention encompasses reagents and strategies for identifying compounds that target specific androgen receptor splice variants, for example, AR-v7, that are important for prostate cancer progression and metastasis, for example in the development of castration-resistant prostate cancer. Also provided herein are specific compounds that modulate the activity of an androgen receptor, or variant thereof, pharmaceutical compositions, and methods of using these compounds and pharmaceutical compositions for modulating the activity of an androgen receptor, or variant thereof in vitro and in vivo.

### BACKGROUND OF THE INVENTION

Prostate cancer is the second leading cause of cancer-related mortality of men in the United States according to the American Cancer Society [Ferlay, J., Autier, P., Boniol, M., Heanue, M., Colombet, M., and Boyle, P. (2007) Estimates of the cancer incidence and mortality in Europe in 2006. Annals of Oncology, 18: 581-92]. A contributing factor is that tumors develop resistance to current treatments, especially in therapies that involve targeting the androgen receptor (AR). Treatments generally rely upon continual androgen deprivation therapy via direct AR antagonism (e.g., enzalutimde, bicalutamide) or decrease in adrenal androgen production (e.g., abiraterone acetate). However, acquired resistance to these treatments potentially leads to castration-resistant prostate cancer (CRPC) and an overall death rate of 1 in 38 men diagnosed with prostate cancer [Cancer Facts & FIGS. 2016: www.cancer.org/acs/groups/cid/documents/webcontent/003134-pdf.pdf; Antonarakis, E. S., Feng, Z., Trock, B. J., Humphreys, E. B., Carducci, M. A., Partin, A. W., Walsh, P. C., and Eisenberger, M. A. (2012) The natural history of metastatic progression in men with prostate-specific antigen recurrence after radical prostatectomy: long-term follow-up. BJU Int, 109: 32-39]. Studies of CRPC demonstrate that despite low levels of circulating androgens, AR-mediated gene expression is often maintained by AR splice variants (AR-vs) that do not rely upon androgen signaling [Yuan, X. and Balk, S. P. (2009) Mechanisms mediating androgen receptor reactivation after castration. Urol Oncol, 27: 36-41]. Due to variations in their C-terminus, these AR-vs are commonly constitutively active and effectively mimic full-length AR (AR-FL) in their ability to transactivate androgen response elements (ARE) without androgen stimulation [Watson, P. A., Arora, V. K., and Sawyers, C. L. (2015) Emerging mechanisms of resistance to androgen receptor inhibitors in prostate cancer. Nat Rev Cancer, 15: 701-711].

AR-FL is a steroid receptor transcription factor composed of a N-terminal domain (NTD), a DNA binding domain (DBD), and a C-terminal domain (CTD) comprising a hinge region and a ligand binding domain (LBD) [Dehm, S. M. and Tindall, D. J. (2007) Androgen receptor structural and functional elements: role and regulation in prostate cancer. Mol Endocrinol, 21: 2855-2863]. In order for AR-FL to translocate to the nucleus to participate in mechanisms controlling gene expression, an androgen binds to the LBD and induces a structural change to expose the nuclear localization signal (NLS), which initiates importation into the nucleus. Drugs such as enzalutamide act in a mechanism that targets the LBD and thus prevents nuclear translocation and AR-mediated gene expression. Androgen receptor splice variants (AR-vs) arise from alternative splicing of the AR gene by insertion of “intronic” cryptic exons downstream of exons that encode the DNA-binding domain, thereby preventing ligand binding domain (LBD) incorporation and resulting in a shortened AR construct characterized by ligand-independent function [Hu, R., Dunn, T. A., Wei, S., Isharwal, S., Veltri, R. W., Humphreys, E., Han, M., Partin, A. W., Vessella, R. L., Isaacs, W. B., et al. (2009) Ligand-independent androgen receptor variants derived from splicing of cryptic exons signify hormone-refractory prostate cancer. Cancer Res, 69: 16-22; Guo, Z., Yang, X., Sun, F., Jiang, R., Linn, D. E., Chen, H., Chen, H., Kong, X., Melamed, J., Tepper, C. G., et al. (2009) A novel androgen receptor splice variant is up-regulated during prostate cancer progression and promotes androgen depletion-resistant growth. Cancer Res, 69: 2305-2313; Dehm, S. M., Schmidt, L. J., Heerners, H. Y., Vessella, R. L., and Tindall, D. J. (2008) Splicing of a novel androgen receptor exon generates a constitutively active androgen receptor that mediates prostate cancer therapy resistance. Cancer Res, 68: 5469-5477]. AR-vs have a basal level of nuclear localization and remain capable of transcriptional activity even in the absence of the LBD [Chan, S. C., Li, Y., and Dehm, S. M. (2012) Androgen receptor splice variants activate androgen receptor target genes and support aberrant prostate cancer cell growth independent of canonical androgen receptor nuclear localization signal. J Biol Chem, 287: 19736-19749]. This loss of the LBD, in turn, provides a resistance pathway for current prostate cancer drug regimens.

Of the 20 known AR-v isoforms, AR-v7 is the most widely identified and clinically important variant in prostate cancer [Sun, S., Sprenger, C. C. T., Vessella, R. L., Haugk, K., Soriano, K., Mostaghel, E. A., Page, S. T., Coleman, I. M., Nguyen, H. M., Sun, H., et al. (2010) Castration resistance in human prostate cancer is conferred by a frequently occurring androgen receptor splice variant. J Clin Invest, 120: 2715-2730; Lu, J., Van der Steen, T., and Tindall, D. J. (2015) Are androgen receptor variants a substitute for the full-length receptor? Nature Rev, 12: 137-144]. AR-v7 is characterized by an unperturbed NTD and DBD with a portion of the NLS and a unique 16-amino acid sequence in its CTD derived from a cryptic exon incorporation [Hu, R., Dunn, T. A., Wei, S., Isharwal, S., Veltri, R. W., Humphreys, E., Han, M., Partin, A. W., Vessella, R. L., Isaacs, W. B., et al. (2009) Ligand-independent androgen receptor variants derived from splicing of cryptic exons signify hormone-refractory prostate cancer. Cancer Res, 69:16-22; Guo, Z., Yang, X., Sun, F., Jiang, R., Linn, D. E., Chen, H., Chen, H., Kong, X., Melamed, J., Tepper, C. G., et al. (2009) A novel androgen receptor splice variant is up-regulated during prostate cancer progression and promotes androgen depletion-resistant growth. Cancer Res, 69: 2305-2313]. AR-v7 is constitutively active in the absence of androgens and can form homodimers and heterodimers with AR-FL to promote canonical AR-mediated gene expression, while also providing an expression profile unique to that of AR-FL, which includes expression of the prostate cancer relevant oncogene AKT1 [Le Page, C., Koumakpayi, I. H., Alam-Fahmy, M., Mes-Masson, A. M., and Saad, F. (2006) Expression and localisation of Akt-1, Akt-2 and Akt-3 correlate with clinical outcome of prostate cancer patients. Br J Cancer, 94: 1906-1912; Ciccarese, C., Santoni, M., Brunelli, M., Buti, S., Modena, A., Nabissi, M., Artibani, W., Martignoni, G., Montironi, R., Tortora, G., et al. (2016) AR-v7 and prostate cancer: the watershed for treatment selection? Cancer Treat Rev, 43: 27-35]. Single-cell RNA-sequence analysis on circulating tumor cells (CTCs) revealed that approximately 43% of CTCs patients with CRPC expressed at least one type of AR splice variant, wherein AR-v7 and ARv567es (AR-v12) were the most prominently expressed [Miyamoto, D. T., Zheng, Y., Wittner, B. S., Lee, R. J., Zhu, H., Broderick, K. T., Desai, R., Fox, D. B., Brannigan, B. W., Trautwein, J., et al. (2015) RNA-Seq of single prostate CTCs implicates noncanonical Wnt signaling in antiandrogen resistance. Science, 349:1351-1356]. Additionally, the AR-v7 expression level in primary tumor cells derived from metastatic prostate cancer and CRPC patients is significantly higher than that in cells derived from patients with localized prostate cancer, and consequently higher AR-v7 expression can be correlated to shorter survival likelihood in CRPC patients (p<0.001) [Qu, Y., Dai, B., Ye, D., Kong, Y., Chang, K., Jia, Z., Yang, X., Zhang, H., Zhu, Y., and Shi, G. (2015) Constitutively active AR-v7 plays an essential role in the development and progression of castration-resistant prostate cancer. Sci Rep, 5: doi: 10.1038/srep07654].

Transcription factors that become overactive in cancers are promising yet untested targets for cancer therapeutics [Koehler A. N. (2010) A complex task? Direct modulation of transcription factors with small molecules. Curr Opin Chem Bio, 14: 331-340; Darnell, J. E. (2002) Transcription factors as targets for cancer therapy. Nat Rev Cancer, 2: 740-749]. These proteins mediate the excessive transcription of genes whose products are required for tumor growth and metastasis. In particular, prostate cancer progression is often driven by deregulation events affecting transcription factors such as ETS (E26 transformation-specific) family members and AR [Baena, E., Shao, Z., Linn, D. E., Glass, K., Hamblen, M. J., Fujiwara, Y., Kim, J., Nguyen, M., Zhang, X., Godinho, F. J., et al. (2013) ETV I directs androgen metabolism and confers aggressive prostate cancer in targeted mice and patients. Genes Dev, 27: 683-698; Chen, Y., Chi, P., Rockowitz, S., laquinta, P. J., Shamu, T., Shukla, S., Gao, D., Sirota, I., Carver, B. S., Wongvipat, J., et al. (2013) ETS factors reprogram the androgen receptor cistrome and prime prostate tumorigenesis in response to PTEN loss. Nat Med, 19: 1023-1029; Dehm, S. M. and Tindall, D. J. (2011) Alternatively spliced androgen receptor variants. Endocr Relat Cancer, 18: R183-R196; Knudsen, K. E. and Penning, T. M. (2010) Partners in crime: deregulation of AR activity and androgen synthesis in prostate cancer. Trends Endocrinol Metab, 21: 315-324]. Directly modulating the function of a transcription factor requires disruption or recruitment of DNA-protein or protein-protein interactions. The discovery or design of small molecules that specifically disrupt or promote these interactions has thus far proven to be a significant challenge, and the protein class is often perceived to be difficult to drug. General and systematic strategies for discovery direct probes of transcription factors remain elusive [Berg T. (2008) Small-molecule inhibitors of protein-protein interactions. Curr Opin Drug Disc Devel, 11: 666-674; Erkizan, H. V., Kong, Y. L., Merchant, M., Schlottmann, S., Barber-Rotenberg, J. S., Yuan, L. S., Abaan, O. D., Chou, T. H., Dakshanamurthy, S., Brown, M. L., et al. (2009) A small molecule blocking oncogenic protein EWS-FL11 interaction with RNA helicase A inhibits growth of Ewing's sarcoma. Nat Med, 15: 750-756; Ng, P. Y., Tang, Y. C., Knosp, W. M., Stadler, H. S., and Shaw, J. T. (2007) Synthesis of diverse lactam carboxamides leading to the discovery of a new transcription-factor inhibitor. Angew Chem Int Ed Engl, 46: 5352-5325; Hammoudeh, D. I., Follis, A. V., Prochownik, E. V., and Metallo, S. J. (2009) Multiple independent binding sites for small-molecule inhibitors on the oncoprotein c-Myc. J Am Chem Soc, 131: 7390-7401]. Due to the limitations of current therapies for treating metastatic and castration-resistant prostate cancer (CRPC), there remains a need for drugs that target AR-vs.

### SUMMARY OF THE INVENTION

The present disclosure provides compounds identified by screening a plurality of compounds for binding to an androgen receptor (e.g., the clinically relevant androgen receptor variant (AR-v) in a given patient). The screen provides the advantage of being able to identify modulators that interact with the domains present in the AR-v promoting resistance to traditional prostate cancer therapies. Despite the fact that many transcription factors (e.g., AR, ETV1, MECP2, FOX family proteins, STAT family proteins, HOX family proteins) are clinically significant due to their association with specific diseases (e.g., diabetes, autoimmune diseases, cancer), it has traditionally been difficult to modulate the function of transcription factors. The screening methods disclosed herein represent a powerful tool for drug discovery of compounds that can modulate the function of proteins that have been traditionally difficult to target. As described herein, the screening methods can be applied to a wide variety of androgen receptor targets, including AR-v7. A number of diseases are associated with aberrant androgen receptor function. The present disclosure provides a method for screening a plurality of compounds against an androgen receptor, or variant thereof.

In one aspect, the present disclosure provides methods of identifying an androgen receptor modulator, the method comprising: (a) exposing a plurality of compounds to an androgen receptor, or variant thereof; (b) selecting one or more compounds that bind to the androgen receptor, or variant thereof; (c) exposing the one or more selected compounds that bind the androgen receptor, or fragment thereof, individually to cells expressing an androgen receptor, or fragment thereof; and (d) selecting one or more compounds that reduce prostate-specific antigen (PSA) expression in the cells. In certain embodiments, the plurality of compounds is provided as a small molecule microarray (SMM). The small molecule microarray may contain approximately 50,000 compounds for screening against the androgen receptor, or variant thereof. In certain embodiments, one or more compounds having an affinity of at least 1 mM for the androgen receptor (e.g., AR-FL, AR-v7), or variant thereof, are selected in step (b) and/or (d). In yet another aspect, step (d) of the method further comprises (e) measuring a first PSA expression level and a second PSA expression level with qPCR, wherein the first PSA expression level is the PSA expression level of cells exposed to a selected compound, and the second PSA expression level is the PSA expression level of untreated cells; (f) comparing said first PSA expression level and said second PSA expression level; and (g) selecting one or more compounds that reduce the first PSA expression level by at least 10% compared to the second PSA expression level.

In one aspect, the present disclosure provides methods of identifying an androgen receptor modulator, the method comprising: (a) contacting a plurality of compounds with an androgen receptor, or variant thereof; (b) selecting one or more compounds that bind to the androgen receptor, or variant thereof; (c) contacting the one or more selected compounds that bind the androgen receptor, or fragment thereof, individually with cells expressing an androgen receptor, or fragment thereof; and (d) selecting one or more compounds that reduce prostate-specific antigen (PSA) expression in the cells. In certain embodiments, the plurality of compounds is provided as a small molecule microarray (SMM). The small molecule microarray may contain approximately 50,000 compounds for screening against the androgen receptor, or variant thereof. In certain embodiments, one or more compounds having an affinity of at least 1 mM for the androgen receptor (e.g., AR-FL, AR-v7), or variant thereof, are selected in step (b) and/or (d). In yet another aspect, step (d) of the method further comprises (e) measuring a first PSA expression level and a second PSA expression level with qPCR, wherein the first PSA expression level is the PSA expression level of cells contacted with a selected compound, and the second PSA expression level is the PSA expression level of untreated cells; (f) comparing said first PSA expression level and said second PSA expression level; and (g) selecting one or more compounds that reduce the first PSA expression level by at least 10% compared to the second PSA expression level.

In another aspect, the present disclosure provides methods of identifying an androgen receptor modulator, the method comprising: (a) exposing a plurality of compounds to an androgen receptor, or variant thereof; (b) selecting one or more compounds that bind to the androgen receptor, or variant thereof; (c) exposing the one or more selected compounds that bind the androgen receptor, or variant thereof, individually to cells expressing: i) an androgen receptor, or variant thereof, and ii) a reporter protein; and (d) selecting one or more compounds that reduce reporter protein activity level. In certain embodiments, the plurality of compounds is provided in a small molecule microarray (SMM). In certain embodiments, one or more compounds having an affinity of at least 1 mM for the androgen receptor (e.g., AR-FL, AR-v7), or variant thereof, are selected in step (b) and/or (d). In yet another aspect, step (d) of the method further comprises (e) measuring a first reporter protein activity level and a second reporter protein activity level, wherein the first reporter protein activity level is the reporter protein activity level of cells treated with a compound selected for binding to an androgen receptor, or variant thereof, and the second reporter protein activity level is the reporter protein activity level of untreated cells, and wherein the first reporter protein activity level and the second reporter activity level are relative luciferase activity; (f) comparing said first reporter protein activity level and said second reporter protein activity level; and (g) selecting one or more compounds that reduce the first reporter protein activity level by at least 10% compared to the second reporter protein activity level. In an embodiment, the reporter protein is a luciferase (e.g., firefly luciferase, Renilla luciferase), beta-galactosidase, or a fluorescent protein (e.g., green fluorescent protein (GFP), red fluorescent protein (RFP), etc.). In an embodiment, the reporter protein is a luciferase. In certain embodiments, the luciferase is firefly luciferase. In certain embodiments, the luciferase is Renilla luciferase. In an embodiment, more than one type of reporter protein may be expressed in a cell (e.g., a reporter system). In an embodiment, the first reporter protein activity level and the second reporter protein activity level are measured using bioluminescence. In certain embodiments, the first reporter protein activity level and the second reporter protein activity level are measured using a bioluminescence signal from luciferase. The relative luciferase activity is the luciferase activity of a first luciferase as compared to the activity of a second luciferase, wherein expression of the first luciferase is under control of the androgen receptor, or variant thereof, and expression of the second luciferase is under control of a constitutively active promoter. In certain embodiments, the first luciferase is firefly luciferase. In certain embodiments, the second luciferase is Renilla luciferase. In certain embodiments, the firefly luciferase is under control of an MMTV promoter that contains one or more androgen response element (ARE). In certain embodiments, the Renilla luciferase is under the control of a constitutively active CMV promoter.

In another aspect, the present disclosure provides methods of identifying an androgen receptor modulator, the method comprising: (a) contacting a plurality of compounds with an androgen receptor, or variant thereof; (b) selecting one or more compounds that bind to the androgen receptor, or variant thereof; (c) contacting the one or more selected compounds that bind the androgen receptor, or variant thereof, individually with cells expressing: i) an androgen receptor, or variant thereof, and ii) a reporter protein; and (d) selecting one or more compounds that reduce reporter protein activity level. In certain embodiments, the plurality of compounds is provided in a small molecule microarray (SMM). In certain embodiments, one or more compounds having an affinity of at least 1 mM for the androgen receptor (e.g., AR-FL, AR-v7), or variant thereof, are selected in step (b) and/or (d). In yet another aspect, step (d) of the method further comprises (e) measuring a first reporter protein activity level and a second reporter protein activity level, wherein the first reporter protein activity level is the reporter protein activity level of cells treated with a compound selected for binding to an androgen receptor, or variant thereof, and the second reporter protein activity level is the reporter protein activity level of untreated cells, and wherein the first reporter protein activity level and the second reporter activity level are relative luciferase activity; (f) comparing said first reporter protein activity level and said second reporter protein activity level; and (g) selecting one or more compounds that reduce the first reporter protein activity level by at least 10% compared to the second reporter protein activity level. In an embodiment, the reporter protein is a luciferase (e.g., firefly luciferase, Renilla luciferase), beta-galactosidase, or a fluorescent protein (e.g., green fluorescent protein (GFP), red fluorescent protein (RFP), etc.). In an embodiment, the reporter protein is a luciferase. In certain embodiments, the luciferase is firefly luciferase. In certain embodiments, the luciferase is Renilla luciferase. In an embodiment, more than one type of reporter protein may be expressed in a cell (e.g., a reporter system). In an embodiment, the first reporter protein activity level and the second reporter protein activity level are measured using bioluminescence. In certain embodiments, the first reporter protein activity level and the second reporter protein activity level are measured using a bioluminescence signal from luciferase. The relative luciferase activity is the luciferase activity of a first luciferase as compared to the activity of a second luciferase, wherein expression of the first luciferase is under control of the androgen receptor, or variant thereof, and expression of the second luciferase is under control of a constitutively active promoter. In certain embodiments, the first luciferase is firefly luciferase. In certain embodiments, the second luciferase is Renilla luciferase. In certain embodiments, the firefly luciferase is under control of an MMTV promoter that contains one or more androgen response element (ARE). In certain embodiments, the Renilla luciferase is under the control of a constitutively active CMV promoter.

In yet another aspect, the present disclosure provides methods of identifying an androgen receptor modulator, the method comprising: (a) exposing a plurality of compounds to an androgen receptor, or variant thereof; (b) selecting one or more compounds that bind to the androgen receptor, or variant thereof; (c) exposing the one or more selected compounds that bind the androgen receptor, or variant thereof, individually to cells expressing an androgen receptor, or variant thereof; (d) selecting one or more compounds that reduce prostate-specific antigen (PSA) expression in the cells; (e) exposing the one or more compounds selected to reduce PSA expression individually to cells expressing: i) an androgen receptor, or variant thereof, and ii) a reporter protein; and (f) selecting one or more compounds that reduce reporter protein activity level. In an embodiment, the androgen receptor (e.g., AR-FL, AR-v7) is provided in the form of purified androgen receptor, or variant thereof. In an embodiment, the androgen receptor (e.g., AR-FL, AR-v7) is provided in the form of a cell lysate comprising the androgen receptor, or variant thereof. In certain embodiments, the plurality of compounds is provided in a small molecule microarray (SMM). In certain embodiments, one or more compounds having an affinity of at least 1 mM for the androgen receptor (e.g., AR-FL, AR-v7), or variant thereof, are selected in step (b), (d), and/or (f). In yet another aspect, step (d) of the method further comprises (g) measuring a first PSA expression level and a second PSA expression level with qPCR, wherein the first PSA expression level is the PSA expression level of cells exposed to a selected compound, and the second PSA expression level is the PSA expression level of untreated cells; (h) comparing said first PSA expression level and said second PSA expression level; and (i) selecting one or more compounds that reduce the first PSA expression level by at least 10% compared to the second PSA expression level. In yet another aspect, step (f) of the method further comprises (j) measuring a first reporter protein activity level and a second reporter protein activity level, wherein the first reporter protein activity level is the reporter protein activity level of cells treated with a compound selected for binding to an androgen receptor, or variant thereof, and the second reporter protein activity level is the reporter protein activity level of untreated cells, and wherein the first reporter protein activity level and the second reporter activity level are relative luciferase activity; (k) comparing said first reporter protein activity level and said second reporter protein activity level; and (l) selecting one or more compounds that reduce the first reporter protein activity level by at least 10% compared to the second reporter protein activity level. In an embodiment, the reporter protein is a luciferase (e.g., firefly luciferase, Renilla luciferase), beta-galactosidase, or a fluorescent protein (e.g., green fluorescent protein (GFP), red fluorescent protein (RFP), etc.). In certain embodiments, the luciferase is firefly luciferase. In certain embodiments, the luciferase is Renilla luciferase. In an embodiment, more than one type of reporter protein may be expressed in a cell (e.g., a reporter system). In an embodiment, the first reporter protein activity level and the second reporter protein activity level are measured using bioluminescence. In certain embodiments, the first reporter protein activity level and the second reporter protein activity level are measured using a bioluminescence signal from luciferase. The relative luciferase activity is the luciferase activity of a first luciferase as compared to the activity of a second luciferase, wherein expression of the first luciferase is under control of the androgen receptor, or variant thereof, and expression of the second luciferase is under control of a constitutively active promoter. In certain embodiments, the first luciferase is firefly luciferase. In certain embodiments, the second luciferase is Renilla luciferase. In certain embodiments, the firefly luciferase is under control of an MMTV promoter that contains one or more androgen response element (ARE). In certain embodiments, the Renilla luciferase is under the control of a constitutively active CMV promoter.

In yet another aspect, the present disclosure provides methods of identifying an androgen receptor modulator, the method comprising: (a) contacting a plurality of compounds with an androgen receptor, or variant thereof; (b) selecting one or more compounds that bind to the androgen receptor, or variant thereof; (c) contacting the one or more selected compounds that bind the androgen receptor, or variant thereof, individually with cells expressing an androgen receptor, or variant thereof; (d) selecting one or more compounds that reduce prostate-specific antigen (PSA) expression in the cells; (e) contacting the one or more compounds selected to reduce PSA expression individually with cells expressing: i) an androgen receptor, or variant thereof, and ii) a reporter protein; and (f) selecting one or more compounds that reduce reporter protein activity level. In an embodiment, the androgen receptor (e.g., AR-FL, AR-v7) is provided in the form of purified androgen receptor, or variant thereof. In an embodiment, the androgen receptor (e.g., AR-FL, AR-v7) is provided in the form of a cell lysate comprising the androgen receptor, or variant thereof. In certain embodiments, the plurality of compounds is provided in a small molecule microarray (SMM). In certain embodiments, one or more compounds having an affinity of at least 1 mM for the androgen receptor (e.g., AR-FL, AR-v7), or variant thereof, are selected in step (b), (d), and/or (f). In yet another aspect, step (d) of the method further comprises (g) measuring a first PSA expression level and a second PSA expression level with qPCR, wherein the first PSA expression level is the PSA expression level of cells contacted with a selected compound, and the second PSA expression level is the PSA expression level of untreated cells; (h) comparing said first PSA expression level and said second PSA expression level; and (i) selecting one or more compounds that reduce the first PSA expression level by at least 10% compared to the second PSA expression level. In yet another aspect, step (f) of the method further comprises (j) measuring a first reporter protein activity level and a second reporter protein activity level, wherein the first reporter protein activity level is the reporter protein activity level of cells treated with a compound selected for binding to an androgen receptor, or variant thereof, and the second reporter protein activity level is the reporter protein activity level of untreated cells, and wherein the first reporter protein activity level and the second reporter activity level are relative luciferase activity; (k) comparing said first reporter protein activity level and said second reporter protein activity level; and (l) selecting one or more compounds that reduce the first reporter protein activity level by at least 10% compared to the second reporter protein activity level. In an embodiment, the reporter protein is a luciferase (e.g., firefly luciferase, Renilla luciferase), beta-galactosidase, or a fluorescent protein (e.g., green fluorescent protein (GFP), red fluorescent protein (RFP), etc.). In certain embodiments, the luciferase is firefly luciferase. In certain embodiments, the luciferase is Renilla luciferase. In an embodiment, more than one type of reporter protein may be expressed in a cell (e.g., a reporter system). In an embodiment, the first reporter protein activity level and the second reporter protein activity level are measured using bioluminescence. In certain embodiments, the first reporter protein activity level and the second reporter protein activity level are measured using a bioluminescence signal from luciferase. The relative luciferase activity is the luciferase activity of a first luciferase as compared to the activity of a second luciferase, wherein expression of the first luciferase is under control of the androgen receptor, or variant thereof, and expression of the second luciferase is under control of a constitutively active promoter. In certain embodiments, the first luciferase is firefly luciferase. In certain embodiments, the second luciferase is Renilla luciferase. In certain embodiments, the firefly luciferase is under control of an MMTV promoter that contains one or more androgen response element (ARE). In certain embodiments, the Renilla luciferase is under the control of a constitutively active CMV promoter.

In another aspect, the present disclosure provides methods for confirming the activity of one or more compounds having an affinity for an androgen receptor, or variant thereof, the methods comprising (a) exposing the one or more selected compounds that bind the androgen receptor, or variant thereof, individually to cells expressing an androgen receptor, or variant thereof; (b) measuring a first PSA expression level and a second PSA expression level with qPCR, wherein the first PSA expression level is the PSA expression level of cells exposed to a selected compound, and the second PSA expression level is the PSA expression level of untreated cells; (c) comparing said first PSA expression level and said second PSA expression level; and (d) determining that the first PSA expression level is lower compared to the second PSA expression level, thereby confirming the activity of one or more compounds. In certain embodiments, one or more compounds that reduce the first PSA expression level by at least 10% compared to the second PSA expression level are considered to be compounds that modulate the activity of the androgen receptor, or variant thereof.

In another aspect, the present disclosure provides methods for confirming the activity of one or more compounds having an affinity for an androgen receptor, or variant thereof, the methods comprising (a) contacting the one or more selected compounds that bind the androgen receptor, or variant thereof, individually with cells expressing an androgen receptor, or variant thereof; (b) measuring a first PSA expression level and a second PSA expression level with qPCR, wherein the first PSA expression level is the PSA expression level of cells contacted with a selected compound, and the second PSA expression level is the PSA expression level of untreated cells; (c) comparing said first PSA expression level and said second PSA expression level; and (d) determining that the first PSA expression level is lower compared to the second PSA expression level, thereby confirming the activity of one or more compounds. In certain embodiments, one or more compounds that reduce the first PSA expression level by at least 10% compared to the second PSA expression level are considered to be compounds that modulate the activity of the androgen receptor, or variant thereof.

In yet another aspect, the present disclosure provides methods for confirming the activity of one or more compounds with an affinity for an androgen receptor, or variant thereof, the methods comprising (a) exposing the one or more selected compounds that bind the androgen receptor, or variant thereof, individually to cells expressing: i) an androgen receptor, or variant thereof, and ii) a reporter protein; (b) measuring a first reporter protein activity level and a second reporter protein activity level, wherein the first reporter protein activity level is the reporter protein activity level of cells treated with a compound selected for binding to an androgen receptor, or variant thereof, and the second reporter protein activity level is the reporter protein activity level of untreated cells, and wherein the first reporter protein activity level and the second reporter activity level are relative luciferase activity; (c) comparing said first reporter protein activity level and said second reporter protein activity level; and (d) determining that the first reporter protein activity level is lower compared to the second reporter protein activity level, thereby confirming the activity of one or more compounds. In certain embodiments, one or more compounds that reduce the first reporter protein activity level by at least 10% compared to the second reporter activity level are considered to be compounds that modulate the activity of the androgen receptor.

In yet another aspect, the present disclosure provides methods for confirming the activity of one or more compounds with an affinity for an androgen receptor, or variant thereof, the methods comprising (a) contacting the one or more selected compounds that bind the androgen receptor, or variant thereof, individually with cells expressing: i) an androgen receptor, or variant thereof, and ii) a reporter protein; (b) measuring a first reporter protein activity level and a second reporter protein activity level, wherein the first reporter protein activity level is the reporter protein activity level of cells treated with a compound selected for binding to an androgen receptor, or variant thereof, and the second reporter protein activity level is the reporter protein activity level of untreated cells, and wherein the first reporter protein activity level and the second reporter activity level are relative luciferase activity; (c) comparing said first reporter protein activity level and said second reporter protein activity level; and (d) determining that the first reporter protein activity level is lower compared to the second reporter protein activity level, thereby confirming the activity of one or more compounds. In certain embodiments, one or more compounds that reduce the first reporter protein activity level by at least 10% compared to the second reporter activity level are considered to be compounds that modulate the activity of the androgen receptor.

Also provided herein are compounds identified using methods provided herein, wherein the compound is a modulator of an androgen receptor, or variant thereof. The compound may be useful in the treatment of proliferative diseases (e.g., cancer (e.g., breast cancer, prostate cancer)).

In one aspect, the present disclosure provides a method of treating a proliferative disease. In certain particular embodiments, the proliferative disease is prostate cancer. In certain embodiments, the compound is a modulator of an androgen receptor. In certain embodiments, the compound is selected from Table 2, infra, and pharmaceutically acceptable salts thereof.

In another aspect, the present disclosure provides a use of a compound, a pharmaceutically acceptable salt thereof, or a pharmaceutical composition therof, in treating a profilerative disease. The proliferative disease may be cancer (e.g., breast cancer, prostate cancer). In certain embodiments, the compound is selected from Table 2, and pharmaceutically acceptable salts thereof.

In certain embodiments, a compound of formula

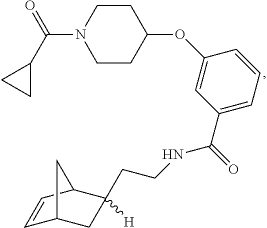 [SMILES: O=C(NCCC1CC2C=CC1C2)c1cccc(OC2CCN(C(=O)C3CC3)CC2)c1]

or a pharmaceutically acceptable salt thereof, for the treatment of prostate cancer is provided. In certain embodiments, a compound of formula

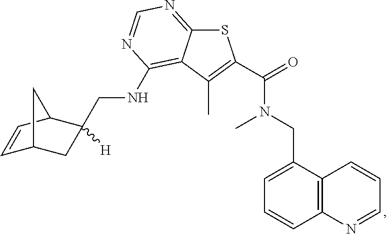 [SMILES: Cc1c(C(=O)N(C)Cc2cccc3ncccc23)sc2ncnc(NCC3CC4C=CC3C4)c12]

or a pharmaceutically acceptable salt thereof, for the treatment of prostate cancer is provided. In one aspect, the present disclosure provides one or more compounds identifed as an androgen receptor modulator, wherein the compounds are selected from Table 2, and pharmaceutically acceptable salts, solvates, hydrates, polymorphs, co-crystals, tautomers, stereoisomers, isotopically labeled derivatives, and prodrugs thereof.

In another aspect, described herein are pharmaceutical compositions comprising a compound described herein, or a pharmaceutically acceptable salt thereof, and optionally a pharmaceutically acceptable excipient. In certain embodiments, the pharmaceutical composition comprises a pharmaceutically acceptable excipitent and a compound selected from Table 2, and pharmaceutically acceptable salts thereof. In an embodiment, a pharmaceutical composition described herein includes a therapeutically or prophylactically effective amount of a compound identified by a method described herein. The pharmaceutical composition may optionally include an additional therapeutic agent. The pharmaceutical compositions may be useful for modulating an androgen receptor (e.g., AR-FL, AR-v7), or variant thereof, in a subject or cell, in treating a disease (e.g., a prostate cancer) in a subject in need thereof, or in preventing a disease in a subject in need thereof. In certain embodiments, the compound being administered or used modulates the activity of the androgen receptor (e.g., AR-FL, AR-v7), or variant thereof. In certain embodiments, the compound being administered or used is an inhibitor or antagonist of the androgen receptor (e.g., AR-FL, AR-v7), or variant thereof. In certain embodiments, the compound modulates gene expression in a subject or a cell, wherein the gene expression is under direct or indirect control of an androgen receptor, or variant thereof.

In one aspect, the present disclosure provides compositions and methods for modulating the activity of an androgen receptor, or variant thereof. In an embodiment, modulation of the activity of the androgen receptor, or variant thereof, comprises inhibiting the androgen receptor, or variant thereof. In certain embodiments, the compositions and methods are useful for modulating an androgen receptor, or variant thereof, wherein the androgen receptor, or variant thereof, is a human full-length androgen receptor (AR-FL) (SEQ ID NO: 1). In certain embodiments, the compositions and methods are useful for modulating an androgen receptor, or variant thereof, wherein the androgen receptor, or variant thereof, is at least 90% identical to the amino acid sequence of AR-FL (SEQ ID NO: 1), or a fragment thereof. In an embodiment, the present disclosure provides compositions and methods useful for modulating the activity of an androgen receptor, or variant thereof, wherein the androgen receptor, or variant thereof, is an androgen receptor variant (AR-v). In certain embodiments, the compositions and methods are useful for modulating an androgen receptor, or variant thereof, wherein the AR-v is one of the 21 known AR-vs, or a fragment thereof. The 21 known AR-vs are AR23, ARQ640X, AR-v1 (AR4), AR-v2, AR-v3 (AR1/2/2b/AR6), AR-v4 (AR1/2/3/2b, AR5), AR-v5, AR-v6, AR-v7 (AR3), AR-v8, AR-v9, AR-v10, AR-v11, AR-v12 (ARV567es), AR-v13, AR-v14, AR-v15, AR-v16, AR-v18, AR8, and AR45. The compositions and methods disclosed herein would also be applicable to any other androgen receptor or variant thereof that is not one of the known 21 AR-vs.

In an embodiment, the present disclosure provides compositions and methods for modulating the activity an androgen receptor, or variant thereof, wherein the androgen receptor, or variant thereof, is AR-v7 (SEQ ID NO: 2). In an embodiment, the compositions and methods are useful for modulating an androgen receptor, or variant thereof, wherein the androgen receptor, or variant thereof, is at least 90% identical to the amino acid sequence of AR-v7 (SEQ ID NO: 2).

In another aspect, the present disclosure provides a method of modulating the activity of an androgen receptor (e.g., AR-FL, AR-v7), or variant thereof, the method comprising exposing the androgen receptor, or variant thereof, to a compound, or pharmaceutically acceptable salt thereof, or pharmaceutical composition thereof, described herein. In an embodiment, the present disclosure provides a method of inhibiting or antagonizing an androgen receptor (e.g., AR-FL, AR-v7), or variant thereof, the method comprising exposing the androgen receptor, or variant thereof, to a compound, or pharmaceutical composition thereof, as described herein.

In another aspect, the present disclosure provides a method of modulating the activity of an androgen receptor (e.g., AR-FL, AR-v7), or variant thereof, the method comprising contacting the androgen receptor, or variant thereof, with a compound, or pharmaceutical composition thereof, described herein. In an embodiment, the present disclosure provides a method of inhibiting or antagonizing an androgen receptor (e.g., AR-FL, AR-v7), or variant thereof, the method comprising contacting the androgen receptor, or variant thereof, with a compound, or pharmaceutical composition thereof, as described herein.

In yet another aspect, the present disclosure provides methods of modulating the activity of an androgen receptor (e.g., AR-FL, AR-v7), or variant thereof, wherein the androgen receptor (e.g., AR-FL, AR-v7), or variant thereof, is submitted in a purified form, the methods comprising exposing the purified form of the androgen receptor (e.g., AR-FL, AR-v7), or variant thereof, to an effective amount of a compound, or a pharmaceutically acceptable salt thereof, or pharmaceutical composition thereof, described herein.

In yet another aspect, the present disclosure provides methods of modulating the activity of an androgen receptor (e.g., AR-FL, AR-v7), or variant thereof, wherein the androgen receptor (e.g., AR-FL, AR-v7), or variant thereof, is submitted in a purified form, the methods comprising contacting the purified form of the androgen receptor (e.g., AR-FL, AR-v7), or variant thereof, with an effective amount of a compound, or pharmaceutical composition thereof, described herein.

In yet another aspect, the present disclosure provides methods of modulating the activity of an androgen receptor (e.g., AR-FL, AR-v7), or variant thereof, in a biological sample, tissue, or cell, the methods comprising exposing the biological sample, tissue, or cell to an effective amount of a compound, or pharmaceutically acceptable salt thereof, or pharmaceutical composition thereof, described herein. In an embodiment, a compound, or pharmaceutically acceptable salt thereof, or pharmaceutical composition thereof, described herein is useful for modulating the activity of an androgen receptor (e.g., AR-FL, AR-v7), or variant thereof, in a prostate gland cell, prostate cancer cell, metastatic prostate cancer cell, castration-resistant prostate cancer cell, or a cell from an established prostate cancer cell line (e.g., LNCaP cell, VCaP cell, or DU145 cell). In certain embodiments, the cell is a metastatic prostate cancer cell. In certain embodiments, the cell is a castration-resistant prostate cancer cell. In certain embodiments, the cell is a LNCaP cell.

In yet another aspect, the present disclosure provides methods of modulating the activity of an androgen receptor (e.g., AR-FL, AR-v7), or variant thereof, in a biological sample, tissue, or cell, the methods comprising contacting the biological sample, tissue, or cell with an effective amount of a compound, or pharmaceutical composition thereof, described herein. In an embodiment, a compound, or pharmaceutical composition thereof, described herein is useful for modulating the activity of an androgen receptor (e.g., AR-FL, AR-v7), or variant thereof, in a prostate gland cell, prostate cancer cell, metastatic prostate cancer cell, castration-resistant prostate cancer cell, or a cell from an established prostate cancer cell line (e.g., LNCaP cell, VCaP cell, or DU145 cell). In certain embodiments, the cell is a metastatic prostate cancer cell. In certain embodiments, the cell is a castration-resistant prostate cancer cell. In certain embodiments, the cell is a LNCaP cell.

In another aspect, the present disclosure provides methods of modulating the activity of an androgen receptor, or variant thereof, the methods comprising exposing a cell expressing the androgen receptor (e.g., AR-FL, AR-v7), or variant thereof, to a compound, or pharmaceutically acceptable salt thereof, or pharmaceutical composition thereof, as described herein. The cell may be present in vivo or in vitro.

In another aspect, the present disclosure provides methods of modulating the activity of an androgen receptor, or variant thereof, the methods comprising contacting a cell expressing the androgen receptor (e.g., AR-FL, AR-v7), or variant thereof, with a compound, or pharmaceutical composition thereof, as described herein. The cell may be present in vivo or in vitro.

In another aspect, the present disclosure provides methods of modulating the expression of a gene in a cell, the methods comprising exposing a cell expressing the androgen receptor (e.g., AR-FL, AR-v7), or variant thereof, to a compound, or pharmaceutically acceptable salt thereof, or pharmaceutical composition thereof, described herein. In an embodiment, the gene may be a gene that is expressed in normal cells or a cancer driver gene. In an embodiment, the gene is any gene that may be expressed differently (e.g., overexpressed, underexpressed) in prostate cancer cells. In certain embodiments, the gene is any gene that may be differentially expressed (e.g., overexpressed or underexpressed) in prostate cancer cells compared to cells with a normal prostate gland cell phenotype (e.g., non-cancerous cells). In certain embodiments, the gene is underexpressed in prostate cancer cells. In certain embodiments, the gene is underexpressed in metastatic prostate cancer cells. In certain embodiments, the gene is underexpressed in castration-resistant prostate cancer cells. In certain embodiments, the gene is overexpressed in prostate cancer cells. In certain embodiments, the gene is overexpressed in metastatic prostate cancer cells. In certain embodiments, the gene is overexpressed in castration-resistant prostate cancer cells. In certain embodiments, the gene that is expressed differentially (e.g., overexpressed or underexpressed) in prostate cancer cells is a gene whose transcription is under control of the androgen receptor, or a variant thereof (e.g., androgen-receptor mediated). In certain embodiments, the gene is overexpressed in prostate cancer cells. In certain embodiments, the gene is overexpressed in castration resistant prostate cancer cells. In certain embodiments, the gene is a gene, wherein the expression of the gene is under control of the androgen receptor, or fragment thereof (e.g., androgen-receptor mediated gene expression). The gene may be under direct control of the androgen receptor (i.e., the gene has androgen response element (ARE) sequence). Alternatively, the gene may be under indirect control of the androgen receptor, where the androgen receptor may activate another protein that in turn activates gene expression. In certain embodiments, the gene is AKT1.

In one aspect, the present disclosure provides methods of modulating the expression of a gene in a cell, the methods comprising contacting a cell expressing the androgen receptor (e.g., AR-FL, AR-v7), or variant thereof, with a compound, or pharmaceutical composition thereof, described herein. In an embodiment, the gene may be a gene that is expressed in normal cells or a cancer driver gene. In an embodiment, the gene is any gene that may be expressed differently (e.g., overexpressed, underexpressed) in prostate cancer cells. In certain embodiments, the gene is any gene that may be differentially expressed (e.g., overexpressed or underexpressed) in prostate cancer cells compared to cells with a normal prostate gland cell phenotype (e.g., non-cancerous cells). In certain embodiments, the gene is underexpressed in prostate cancer cells. In certain embodiments, the gene is underexpressed in metastatic prostate cancer cells. In certain embodiments, the gene is underexpressed in castration-resistant prostate cancer cells. In certain embodiments, the gene is overexpressed in prostate cancer cells. In certain embodiments, the gene is overexpressed in metastatic prostate cancer cells. In certain embodiments, the gene is overexpressed in castration-resistant prostate cancer cells. In certain embodiments, the gene that is expressed differentially (e.g., overexpressed or underexpressed) in prostate cancer cells is a gene whose transcription is under control of the androgen receptor, or a variant thereof (e.g., androgen-receptor mediated). In certain embodiments, the gene is overexpressed in prostate cancer cells. In certain embodiments, the gene is overexpressed in castration resistant prostate cancer cells. In certain embodiments, the gene is a gene, wherein the expression of the gene is under control of the androgen receptor, or fragment thereof (e.g., androgen-receptor mediated gene expression). The gene may be under direct control of the androgen receptor (i.e., the gene has androgen response element (ARE) sequence). Alternatively, the gene may be under indirect control of the androgen receptor, where the androgen receptor may activate another protein that in turn activates gene expression. In certain embodiments, the gene is AKT1.

In another aspect, the present disclosure provides methods of modulating the activity of an androgen receptor (e.g., AR-FL, AR-v7), or a variant thereof, in a subject in need thereof, the methods comprising administering to the subject a therapeutically effective amount of a compound, or pharmaceutical composition thereof, as described herein. In certain embodiments, the subject is a subject with cancer (e.g., breast cancer, prostate cancer). In certain embodiments, the subject is a subject with prostate cancer. In certain embodiments, the subject is a subject with metastatic prostate cancer. In certain embodiments, the subject is a subject with castration-resistant prostate cancer (CRPC). The subject may be a mammal (e.g., human). The subject may be a male human.

In one aspect, the present disclosure provides compounds that may be used as a targeting moiety to target a target of interest (e.g., a protein). In certain embodiments, the compounds target the androgen receptor, or variant thereof. In certain embodiments, the compounds target the androgen receptor, or variant thereof, wherein the androgen receptor is AR-FL. In certain embodiments, the compounds target the androgen receptor, or variant thereof, wherein the androgen receptor is AR-v7. In certain embodiments, the compounds may be attached to a linker of any length and composition. In certain embodiments, the compounds may be attached to a linker of any length and composition, wherein the linker is further attached to a molecule of interest (e.g., an agent useful for diagnostic purposes). In certain embodiments, the compounds may be attached to a linker of any length and composition, wherein the linker is further attached to a diagnostic agent (e.g., a molecule used to detect a disease or abnormal function in a subject). Thus the compound may be used to identify the presence of AR-vs in a subject. The compound may also be used to deliver a therapeutic agent to a cell expressing an androgen receptor, or variant thereof, for example, a prostate cancer cell expressing AR-v7.

In another aspect, the present disclosure provides methods for diagnosing a patient, the methods comprising administering to the subject a therapeutically effective amount of a compound, or pharmaceutical composition thereof, described herein, wherein the compound is a compound comprising a targeting moiety that targets the androgen receptor, or variant thereof.

In certain embodiments, the compound being administered or used selectively modulates the activity of an androgen receptor, or variant thereof. In certain embodiments, the compound being administered or used selectively modulates the activity of an androgen receptor variant (e.g., AR-v7). When a compound, pharmaceutical composition, method, use, or kit is referred to as “selectively,” “specifically,” or “competitively” modulating the activity of a target (e.g., a protein), the compound, pharmaceutical composition, method, use, or kit modulates the activity of a particular target (e.g., a target protein) to a greater extent (e.g., not less than 2-fold, not less than 5-fold, not less than 10-fold, not less than 30-fold, not less than 100-fold, not less than 1,000-fold, or not less than 10,000-fold; and/or: not more than 2-fold, not more than 5-fold, not more than 10-fold, not more than 30-fold, not more than 100-fold, not more than 1,000-fold, or not more than 10,000-fold) than another protein.

The practice of certain aspects of the present invention may employ conventional techniques of molecular biology, cell culture, recombinant nucleic acid (e.g., DNA) technology, immunology, transgenic biology, microbiology, nucleic acid and polypeptide synthesis, detection, manipulation, and quantification, that are within the ordinary skill of the art. See, e.g., Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2012; Sambrook, Russell, and Sambrook, Molecular Cloning: A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988. Information regarding diagnosis and treatments of various diseases, including prostate cancer and related cancers, is found in Longo, D., et al. (eds.), Harrison's Principles of Internal Medicine, 18^{th} Ed.; McGraw-Hill Professional, 2011. Information regarding various therapeutic agents and human diseases, including cancer, is found in Brunton, L., et al. (eds.) Goodman and Gilman's The Pharmacological Basis of Therapeutics, 12^{th} Ed., McGraw Hill, 2010 and/or Katzung, B. (ed.) Basic and Clinical Pharmacology, McGraw-Hill/Appleton & Lange; 11^{th} edition (July 2009). All patents, patent applications, books, articles, documents, databases, websites, publications, references, etc., mentioned herein are incorporated by reference in their entirety. In case of a conflict between the specification and any of the incorporated references, the specification (including any amendments thereof), shall control. Applicants reserve the right to amend the specification based, e.g., on any of the incorporated material and/or to correct obvious errors. None of the content of the incorporated material shall limit the invention. Standard art-accepted meanings of terms are used herein unless indicated otherwise. Standard abbreviations for various terms are used herein.

The details of certain embodiments of the invention are set forth in the Detailed Description of Certain Embodiments, as described below. Other features, objects, and advantages of the invention will be apparent from the Definitions, Examples, Figures, and Claims.

### Definitions

Descriptions and certain information relating to various terms used in the present disclosure are collected herein for convenience.

The term “agent” is used herein to refer to any substance, compound (e.g., molecule), supramolecular complex, material, or combination or mixture thereof. A compound may be any agent that can be represented by a chemical formula, chemical structure, or sequence. Example of agents, include, e.g., small molecules, polypeptides, nucleic acids (e.g., RNAi agents, antisense oligonucleotide, aptamers), lipids, polysaccharides, etc. In general, agents may be obtained using any suitable method known in the art. The ordinary skilled artisan will select an appropriate method based, e.g., on the nature of the agent. An agent may be at least partly purified. In some embodiments an agent may be provided as part of a composition, which may contain, e.g., a counter-ion, aqueous or non-aqueous diluent or carrier, buffer, preservative, or other ingredient, in addition to the agent, in various embodiments. In some embodiments an agent may be provided as a salt, ester, hydrate, or solvate. In some embodiments an agent is cell-permeable, e.g., within the range of typical agents that are taken up by cells and acts intracellularly, e.g., within mammalian cells, to produce a biological effect. Certain compounds may exist in particular geometric or stereoisomeric forms. Such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof are encompassed by this disclosure in various embodiments unless otherwise indicated. Certain compounds may exist in a variety or protonation states, may have a variety of configurations, may exist as solvates [e.g., with water (i.e. hydrates) or common solvents] and/or may have different crystalline forms (e.g., polymorphs) or different tautomeric forms. Embodiments exhibiting such alternative protonation states, configurations, solvates, and forms are encompassed by the present disclosure where applicable.

The terms “assess,” “determine,” “evaluate,” and “assay” are used interchangeably herein to refer to any form of detection or measurement, and include determining whether a substance, signal, disease, condition, etc., is present or not. The result of an assessment may be expressed in qualitative and/or quantitative terms. Assessing may be relative or absolute. “Assessing the presence of” includes determining the amount of something that is present or determining whether it is present or absent.

“Identity” or “percent identity” is a measure of the extent to which the sequence of two or more nucleic acids or polypeptides is the same. The percent identity between a sequence of interest A and a second sequence B may be computed by aligning the sequences, allowing the introduction of gaps to maximize identity, determining the number of residues (nucleotides or amino acids) that are opposite an identical residue, dividing by the minimum of TG_{A} and TG_{B}(here TG_{A} and TG_{B} are the sum of the number of residues and internal gap positions in sequences A and B in the alignment), and multiplying by 100. When computing the number of identical residues needed to achieve a particular percent identity, fractions are to be rounded to the nearest whole number. Sequences can be aligned with the use of a variety of computer programs known in the art. For example, computer programs such as BLAST2, BLASTN, BLASTP, Gapped BLAST, etc., may be used to generate alignments and/or to obtain a percent identity. The algorithm of Karlin and Altschul (Karlin and Altschul, Proc Natl Acad Sci USA, 87: 22264-2268, 1990) modified as in Karlin and Altschul, Proc Natl Acad Sci USA, 90: 5873-5877, 1993 is incorporated into the NBLAST and XBLAST programs of Altschul et al. [Altschul, et al. (1990) J Mol Biol, 215: 403-410]. In some embodiments, to obtain gapped alignments for comparison purposes, Gapped BLAST is utilized as described in Altschul et al. [Altschul, et al. (1997) Nucleic Acids Res, 25: 3389-3402]. When utilizing BLAST and Gapped BLAST programs, the default parameters of the respective programs may be used. See the Web site having URL www.ncbi.nlm.nih.gov and/or McGinnis, S. and Madden, T L, W20-W25 Nucleic Acids Research, 2004, Vol. 32, Web server issue. Other suitable programs include CLUSTALW [Thompson, J. D., Higgins, D. G., and Gibson, T. J. (1994) Nuc Acid Res, 22: 4673-4680], CLUSTAL Omega [Sievers, F., Wilm, A., Dineen, D., et al. (2011) Fast, scalable generation of high-quality protein multiple sequence alignments using Clustal Omega. Mol Sys Biol, 7: doi:10.1038/msb.2011.75], and GAP (GCG Version 9.1; which implements the Needleman & Wunsch, 1970 algorithm [Needleman, S. B. and Wunsch, C. D. (1970) J. Mol Biol, 48: 443-453]). Percent identity may be evaluated over a window of evaluation. In some embodiments a window of evaluation may have a length of at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more, e.g., 100%, of the length of the shortest of the sequences being compared. In some embodiments a window of evaluation is at least 100; 200; 300; 400; 500; 600; 700; 800; 900; 1,000; 1,200; 1,500; 2,000; 2,500; 3,000; 3,500; 4,000; 4,500; or 5,000 amino acids. In some embodiments no more than 20%, 10%, 5%, or 1% of positions in either sequence or in both sequences over a window of evaluation are occupied by a gap. In some embodiments no more than 20%, 10%, 5%, or 1% of positions in either sequence or in both sequences are occupied by a gap.

“Detection reagent” refers to an agent that is useful to specifically detect a gene product, protein, or other analyte of interest, e.g., an agent that specifically binds to the gene product, protein, or other analyte. Examples of agents useful as detection reagents include, e.g., nucleic acid probes or primers that hybridize to RNA or DNA to be detected, antibodies, aptamers, or small molecule ligands that bind to polypeptides to be detected, and the like. In some embodiments a detection reagent comprises a label. In some embodiments a detection reagent is attached to a support. Such attachment may be covalent or noncovalent in various embodiments. Methods suitable for attaching detection reagents or analytes to supports will be apparent to those of ordinary skill in the art. A support may be a substantially planar or flat support or may be a particulate support, e.g., an approximately spherical support such as a microparticle (also referred to as a “bead”, “microsphere”), nanoparticle (or like terms), or population of microparticles. In some embodiments a support is a slide, chip, or filter. In some embodiments a support is at least a portion of an inner surface of a well or other vessel, channel, flow cell, or the like. A support may be rigid, flexible, solid, or semi-solid (e.g., gel). A support may be comprised of a variety of materials such as, for example, glass, quartz, plastic, metal, silicon, agarose, nylon, or paper. A support may be at least in part coated, e.g., with a polymer or substance comprising a reactive functional group suitable for attaching a detection reagent or analyte thereto. The term “detecting” encompasses any method that involves a detecting agent and any gene product, protein, or other analyte of interest.

An “effective amount” or “effective dose” of an agent (or composition containing such agent) refers to the amount sufficient to achieve a desired biological and/or pharmacological effect, e.g., when delivered to a cell or organism according to a selected administration form, route, and/or schedule. The phrases “effective amount” and “therapeutically effective amount” are used interchangeably. As will be appreciated by those of ordinary skill in this art, the absolute amount of a particular agent or composition that is effective may vary depending on such factors as the desired biological or pharmacological endpoint, the agent to be delivered, the target tissue, etc. Those of ordinary skill in the art will further understand that an “effective amount” may be exposed to cells or administered to a subject in a single dose, or through use of multiple doses, in various embodiments.

An “effective amount” or “effective dose” of an agent (or composition containing such agent) refers to the amount sufficient to achieve a desired biological and/or pharmacological effect, e.g., when delivered to a cell or organism according to a selected administration form, route, and/or schedule. The phrases “effective amount” and “therapeutically effective amount” are used interchangeably. As will be appreciated by those of ordinary skill in this art, the absolute amount of a particular agent or composition that is effective may vary depending on such factors as the desired biological or pharmacological endpoint, the agent to be delivered, the target tissue, etc. Those of ordinary skill in the art will further understand that an “effective amount” may be contacted with cells or administered to a subject in a single dose, or through use of multiple doses, in various embodiments.

The term “expression” encompasses the processes by which nucleic acids (e.g., DNA) are transcribed to produce RNA, and (where applicable) RNA transcripts are processed and translated into polypeptides.

“Nucleic acid” is used interchangeably with “polynucleotide” and encompasses polymers of nucleotides. “Oligonucleotide” refers to a relatively short nucleic acid, e.g., typically between about 4 and about 100 nucleotides (nt) long, e.g., between 8-60 nt or between 10-40 nt long. Nucleotides include, e.g., ribonucleotides or deoxyribonucleotides. In some embodiments a nucleic acid comprises or consists of deoxyribonucleic acid (DNA) or ribonucleic acid (RNA). In some embodiments a nucleic acid comprises or includes only standard nucleobases (often referred to as “bases”). The standard bases are cytosine, guanine, adenine (which are found in DNA and RNA), thymine (which is found in DNA) and uracil (which is found in RNA), abbreviated as C, G, A, T, and U, respectively. In some embodiments a nucleic acid may comprise one or more non-standard nucleobases, which may be naturally occurring or non-naturally occurring (i.e., artificial; not found in nature) in various embodiments. In some embodiments a nucleic acid may comprise one or more chemically or biologically modified bases (e.g., alkylated or methylated bases), modified sugars [e.g., 2′-O-alkyribose (e.g., 2′-O methylribose), 2′-fluororibose, arabinose, or hexose], modified phosphate groups or modified internucleoside linkages (i.e., a linkage other than a phosphodiester linkage between consecutive nucleosides, e.g., between the 3′ carbon atom of one sugar molecule and the 5′ carbon atom of another), such as phosphorothioates, 5′-N-phosphoramidites, alkylphosphonates, phosphorodithioates, phosphate esters, alkylphosphonothioates, phosphoramidates, carbamates, carbonates, phosphate triesters, acetamidates, carboxymethyl esters and peptide bonds). In some embodiments a modified base has a label (e.g., a small organic molecule such as a fluorophore dye) covalently attached thereto. In some embodiments the label or a functional group to which a label can be attached is incorporated or attached at a position that is not involved in Watson-Crick base pairing such that a modification at that position will not significantly interfere with hybridization. For example the C-5 position of UTP and dUTP is not involved in Watson-Crick base-pairing and is a useful site for modification or attachment of a label. Modifications may occur anywhere in a nucleic acid. A “modified nucleic acid” is a nucleic acid that may be modified throughout part or all of its length, may contain alternating modified and unmodified nucleotides or internucleoside linkages, or may contain one or more segments of unmodified nucleic acid and one or more segments of modified nucleic acid. A modified nucleic acid may contain multiple different modifications, which may be of different types. A modified nucleic acid may have increased stability (e.g., decreased susceptibility to spontaneous or nuclease-catalyzed hydrolysis) or altered hybridization properties (e.g., increased affinity or specificity for a target, e.g., a complementary nucleic acid), relative to an unmodified counterpart having the same nucleobase sequence. In some embodiments a modified nucleic acid comprises a modified nucleobase having a label covalently attached thereto. Non-standard nucleotides and other nucleic acid modifications known in the art as being useful in the context of nucleic acid detection reagents, RNA interference (RNAi), aptamer, or antisense-based molecules for research or therapeutic purposes are contemplated for use in various embodiments of the instant invention. See, e.g., The Molecular Probes® Handbook—A Guide to Fluorescent Probes and Labeling Technologies (cited above), Bioconjugate Techniques (cited above), Crooke, S. T. (ed.) Antisense drug technology: principles, strategies, and applications, Boca Raton: CRC Press, 2008; Kurrcek, J. (ed.) Therapeutic oligonucleotides, RSC biomolecular sciences. Cambridge: Royal Society of Chemistry, 2008. A nucleic acid can be single-stranded, double-stranded, or partially double-stranded. An at least partially double-stranded nucleic acid can have one or more overhangs, e.g., 5′ and/or 3′ overhang(s). Where a nucleic acid sequence is disclosed herein, it should be understood that its complement and double-stranded form is also disclosed.

The term “gene” refers to a locus (e.g., region) of DNA that is comprised of nucleotides. Generally, a gene contains multiple regions, including one or more upstream or downstream regulatory sequences (e.g., enhancer/silencer, promoter, 5′ non-coding sequences, 3′ non-coding sequences) that is normally required to initiate transcription, an open reading frame comprising one or more exons and one or more introns. An “exon” is any part of a gene that will encode part of the final mature RNA, which will be translated into a protein sequence. An “intron” is any part of a gene that is removed by RNA splicing during maturation of the final mature RNA. A “cryptic exon” is an exon that can introduce a premature translation stop codon into mature RNA or result in atypical splicing patterns. The term “gene” may refer to a nucleic acid fragment that expresses a protein, including regulatory sequences preceding (5′ non-coding sequences) and following (3′ non-coding sequences) the coding sequence. “Native gene” refers to a gene as found in nature with its own regulatory sequences. “Chimeric gene” or “chimeric construct” refers to any gene or a construct, not a native gene, comprising regulatory and coding sequences that are not found together in nature. Accordingly, a chimeric gene or chimeric construct may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but which is introduced into the host organism by gene transfer. Foreign genes can comprise native genes inserted into a non-native organism, or chimeric genes. A “transgene” is a gene that has been introduced into the genome by a transformation procedure.

A “hormone response element” is a short nucleotide sequence (e.g., DNA sequence) within the promoter region of a gene that is capable of interacting with (e.g., binding) a specific hormone receptor complex and thus regulate transcription and gene expression. A gene may have one hormone response element, or more than one different hormone response elements for complex control over transcription and gene expression. An androgen respose element (ARE) is capable of interacting with the androgen receptor, or variant thereof.

“Phenotypic assays” or “phenotypic screens” (used interchangeably herein) are used to identify or confirm that a compound (e.g., small molecule, peptide, RNAi, etc.) alters the phenotype of a cell or organism. A phenotype is any observable characteristic or trait (e.g., morphology, development, biochemical properties, physiological properties, etc.) or the composite thereof of an organism. An organism's phenotype results from the expression of its genetic code, i.e., its genotype. An organisms phenotype may also be influenced by environmental factors. In certain embodiments, compounds that cause a desirable change in phenotype are identified by any of the methods disclosed herein.

“Gain of function” generally refers to acquisition of a new, altered, and/or abnormal function or increased function as compared with a reference. The reference may be, e.g., a level or average level of function possessed by a normal gene product (e.g., a gene product whose sequence is the same as a reference sequence) or found in healthy cell(s) or subject(s). An average may be taken across any number of values. In certain embodiments the reference level may be the upper limit of a reference range. In certain embodiments the function may be increased by at least 10%, 20%, 30%, 40%, 50%, 60%, 70%, 80%, 90%, 95%, 99%, or 100% of the reference level. In certain embodiments the function may be increased by between 1 to 2-fold, 2 to 5-fold, 5 to 10-fold, 10 to 20-fold, 20 to 50-fold, 50 to 100-fold, or more, of the reference level. In certain embodiments the function may be increased to a level or within a range that has a statistically significant correlation with or demonstrated causative relationship with a neurodegenerative disease. A “gain of function” mutation in a gene results in a change in a gene product of the gene or increases the expression level of the gene product, such that it gains a new and abnormal function or an abnormally increased function as compared with a gene product of a normal gene. The function may be new in that it is distinct from the activit(ies) of the normal gene product or may result from an increase in or dysregulation of a normal activity of the gene product. The altered gene product encoded by a gene harboring a gain of function mutation may, for example, have one or more altered residues that causes the gene product to have the ability to interact with different cellular molecules or structures than does the normal gene product or causes the gene product to be mislocalized or dysregulated. For purposes hereof, gain of function mutations encompass dominant negative mutations. Dominant negative mutations result in an altered gene product that lacks a function of the normal gene product and acts antagonistically to the normal gene product by, for example, competing with the normal gene product in a context such as a binding partner, ligand, component of a multimolecular complex (e.g., an oligomer), or substrate but failing to fulfill the normal function of the gene product in that context. The altered gene product encoded by a gene harboring a dominant negative mutation may, for example, be a truncated or otherwise altered form of the normal gene product that retains sufficient structure to compete with the normal gene product. In some embodiments a phenotype or disease resulting from a gain of function mutation in a diploid cell or organism has an autosomal dominant inheritance pattern. A “function” may be any biological activity of a gene product. A biological activity may be, for example, catalyzing a particular reaction, binding to or transporting a particular molecule or complex, participating in or interfering with a biological process carried out by a cell or cells or within a subject, etc. The particular function(s) resulting from a gain of function mutation or lost due to a loss of function mutation may or may not be known.

The term “gene product” (also referred to herein as “gene expression product” or “expression product”) encompasses products resulting from expression of a gene, such as RNA transcribed from a gene and polypeptides arising from translation of such RNA. It will be appreciated that certain gene products may undergo processing or modification, e.g., in a cell. For example, RNA transcripts may be spliced, polyadenylated, etc., prior to mRNA translation, and/or polypeptides may undergo co-translational or post-translational processing such as removal of secretion signal sequences, removal of organelle targeting sequences, or modifications such as phosphorylation, fatty acylation, etc. The term “gene product” encompasses such processed or modified forms. Genomic, mRNA, polypeptide sequences from a variety of species, including human, are known in the art and are available in publicly accessible databases such as those available at the National Center for Biotechnology Information (www.ncbi.nih.gov) or Universal Protein Resource (www.uniprot.org). Databases include, e.g., GenBank, RefSeq, Gene, UniProtKB/SwissProt, UniProtKB/Trembl, and the like. In general, sequences, e.g., mRNA and polypeptide sequences, in the NCBI Reference Sequence database may be used as gene product sequences for a gene of interest. It will be appreciated that multiple alleles of a gene may exist among individuals of the same species. For example, differences in one or more nucleotides (e.g., up to about 1%, 2%, 3-5% of the nucleotides) of the nucleic acids encoding a particular protein may exist among individuals of a given species. Due to the degeneracy of the genetic code, such variations often do not alter the encoded amino acid sequence, although DNA polymorphisms that lead to changes in the sequence of the encoded proteins can exist. Examples of polymorphic variants can be found in, e.g., the Single Nucleotide Polymorphism Database (dbSNP), available at the NCBI website at www.ncbi.nlm.nih.gov/projects/SNP/ [Sherry, S. T., et al. (2001) dbSNP: The NCBI database of genetic variation. Nucl Acids Res, 29: 308-311; Kitts, A. and Sherry, S. (2009) The single nucleotide polymorphism database (dbSNP) of nucleotide sequence variation. In: The NCBI Handbook (Internet); McEntyre, J., Ostell, J., editors. Bethesda (Md.): National Center for Biotechnology Information (US); 2002 (www.ncbi.nlm.nih.gov/bookshelf/br.fcgi?book=handbook&part=ch5)]. Multiple isoforms of certain proteins may exist, e.g., as a result of alternative RNA splicing or editing. In general, where aspects of this disclosure pertain to a gene or gene product, embodiments pertaining to allelic variants or isoforms are encompassed, if applicable, unless indicated otherwise. Certain embodiments may be directed to particular sequence(s), e.g., particular allele(s) or isoform(s).

The term “splice variant” and “split variant” are used interchangeably herein. A splice variant is any protein product that arises from alternative or differential splicing of a gene. Alternative splicing is the process of including or excluding a particular exon from the final mature and processed messenger RNA (mRNA) produced from that gene. Splice variants may also be referred to as “isoforms” of a particular protein.

The term “label” (also referred to as “detectable label”) refers to any moiety that facilitates detection and, optionally, quantification, of an entity that comprises it or to which it is attached. The term “label” is used interchangeably with “tag” herein, unless otherwise explicitly stated. In general, a label may be detectable by, e.g., spectroscopic, photochemical, biochemical, immunochemical, electrical, optical, chemical or other means. In some embodiments a detectable label produces an optically detectable signal (e.g., emission and/or absorption of light), which can be detected e.g., visually or using suitable instrumentation such as a light microscope, a spectrophotometer, a fluorescence microscope, a fluorescent sample reader, a fluorescence activated cell sorter, a camera, or any device containing a photodetector. Labels that may be used in various embodiments include, e.g., organic materials (including organic small molecule fluorophores (sometimes termed “dyes”); quenchers (e.g., dark quenchers), polymers; fluorescent proteins; enzymes; inorganic materials such as metal chelates, metal particles, colloidal metal, metal and semiconductor nanocrystals (e.g., quantum dots); compounds that exhibit luminescence upon enzyme-catalyzed oxidation such as naturally occurring or synthetic luciferins (e.g., firefly luciferin or coelenterazine and structurally related compounds); haptens (e.g., biotin, dinitrophenyl, digoxigenin); radioactive atoms (e.g., radioisotopes such as ³H, ¹⁴C, ³²P, ³³P, ³⁵S, ¹²⁵I), stable isotopes (e.g., ¹³C, ²H); magnetic or paramagnetic molecules or particles, etc. Fluorescent dyes include, e.g., acridine dyes; BODIPY, coumarins, cyanine dyes, napthalenes (e.g., dansyl chloride, dansyl amide), xanthene dyes (e.g., fluorescein, rhodamines), and derivatives of any of the foregoing. Examples of fluorescent dyes include Cy3, Cy3.5, Cy5, Cy5.5, Cy7, Alexa® Fluor dyes, DyLight® Fluor dyes, FITC, TAMRA, Oregon Green dyes, Texas Red, among others. Fluorescent proteins include green fluorescent protein (GFP), blue, sapphire, yellow, red, orange, and cyan fluorescent proteins and fluorescent variants such as enhanced GFP (eGFP), mFruits such as mCherry, mTomato, mStrawberry, R-Phycoerythrin, among others. Enzymes useful as labels include, e.g., enzymes that act on a substrate to produce a colored, fluorescent, or luminescent substance. Examples include luciferases, beta-galactosidase, horseradish peroxidase, and alkaline phosphatase. Luciferases include those from various insects (e.g., fireflies, beetles) and marine organisms [e.g., cnidaria such as Renilla (e.g., Renilla reniformis), copepods such as Gaussia (e.g., Gaussia princeps) or Metridia (e.g., Metridia longa, Metridia pacifica), and modified versions of the naturally occurring proteins]. A wide variety of systems for labeling and/or detecting labels or labeled entities are known in the art.

Numerous detectable labels and methods for their use, detection, modification, and/or incorporation into or conjugation (e.g., covalent or noncovalent attachment) to biomolecules such as nucleic acids or proteins, etc., are described in Johnson, I. and Spence, M. T. Z. (Eds.), The Molecular Probes® Handbook—A Guide to Fluorescent Probes and Labeling Technologies. 11^{th} edition (Life Technologies/Invitrogen Corp.) available online on the Life Technologies website at www.invitrogen.com/site/us/en/home/References/Molecular-Probes-The-Handbook.html and Hermanson, G. T., Bioconjugate Techniques, 2^{nd} ed., Academic Press (2008). Many labels are available as derivatives that are attached to or incorporate a reactive functional group so that the label can be conveniently conjugated to a biomolecule or other entity of interest that comprises an appropriate second functional group (which second functional group may either occur naturally in the biomolecule or may be introduced during or after synthesis). For example, an active ester (e.g., a succinimidyl ester), carboxylate, isothiocyanate, or hydrazine group can be reacted with an amino group; a carbodiimide can be reacted with a carboxyl group; a maleimide, iodoacetamide, or alkyl bromide (e.g., methyl bromide) can be reacted with a thiol (sulfhydryl); an alkyne can be reacted with an azide (via a click chemistry reaction such as a copper-catalyzed or copper-free azide-alkyne cycloaddition). Thus, for example, an N-hydroxysuccinide (NHS)-functionalized derivative of a fluorophore or hapten (such as biotin) can be reacted with a primary amine such as that present in a lysine side chain in a protein or in an aminoallyl-modified nucleotide incorporated into a nucleic acid during synthesis. A label may be directly attached to an entity or may be attached to an entity via a spacer or linking group, e.g., an alkyl, alkylene, aminoallyl, aminoalkynyl, or oligoethylene glycol spacer or linking group, which may have a length of, e.g., between 1 and 4, 4-8, 8-12, 12-20 atoms, or more in various embodiments. A label or labeled entity may be directly detectable or indirectly detectable in various embodiments. A label or labeling moiety may be directly detectable (i.e., it does not require any further reaction or reagent to be detectable, e.g., a fluorophore is directly detectable) or it may be indirectly detectable (e.g., it is rendered detectable through reaction or binding with another entity that is detectable, e.g., a hapten is detectable by immunostaining after reaction with an appropriate antibody comprising a reporter such as a fluorophore or enzyme; an enzyme acts on a substrate to generate a directly detectable signal). A label may be used for a variety of purposes in addition to or instead of detecting a label or labeled entity. For example, a label can be used to isolate or purify a substance comprising the label or having the label attached thereto. In some embodiments, the label is an epitope tag, e.g., hemagglutinin (HA) is a surface glycoprotein that facilitates detection, selection, isolation, and purification of the entity (e.g., protein) that is physically associated with HA. In certain embodiments, HA is encoded in an expression vector comprising an entity of interest, such that the entity is expressed as a fusion protein with HA (e.g., HA-tagged). The term “labeled” is used herein to indicate that an entity (e.g., a molecule, probe, cell, tissue, etc.) comprises or is physically associated with (e.g., via a covalent bond or noncovalent association) a label, such that the entity can be detected. In some embodiments a detectable label is selected such that it generates a signal that can be measured and whose intensity is related to (e.g., proportional to) the amount of the label. In some embodiments two or more different labels or labeled entities are used or present in a composition. In some embodiments the labels may be selected to be distinguishable from each other. For example, they may absorb or emit light of different wavelengths. In some embodiments the labels may be selected to interact with each other. For example, a first label may be a donor molecule that transfers energy to a second label, which serves as an acceptor molecule through nonradiative dipole-dipole coupling as in resonance energy transfer (RET), e.g., Firster resonance energy transfer (FRET, also commonly called fluorescence resonance energy transfer),

“Modulate” as used herein means to decrease (e.g., inhibit, reduce, suppress) or increase (e.g., stimulate, activate, enhance) a level, response, property, activity, pathway, or process. A “modulator” is an agent capable of modulating a level, response, property, activity, pathway, or process. A modulator may be an inhibitor, antagonist, activator, or agonist. In some embodiments modulation may refer to an alteration, e.g., inhibition or increase, of the relevant level, response, property, activity, pathway, or process by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99%.

“Expose,” “exposing,” and similar terms as used herein in the context of a biological receptor (e.g., an androgen receptor or variant thereof) refers to subjecting the biological receptor, or allowing the biological receptor to be subjected to an action, influence, or condition, e.g., the direct or indirect action or influence of a compound of the invention, or a condition created or maintained directly or indirectly by a compound of the invention.

“Contact,” “contacting,” and similar terms as used herein may refer to either direct or indirect contact, or both.

“Bind”, “binding,” and similar terms as used herein may refer to a direct interaction between a compound and a receptor (e.g., an androgen receptor), or fragment thereof, or an indirect interaction, for example, involving one or more androgen receptor interactome partners.

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. In some embodiments, a protein comprises a homodimer or a heterodimer. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain (e.g., the gRNA binding domain of Cas9 that directs the binding of the protein to a target site) and a nucleic acid cleavage domain. In some embodiments, a protein comprises a proteinaceous part, e.g., an amino acid sequence constituting a nucleic acid binding domain, and an organic compound, e.g., a compound that can act as a nucleic acid cleavage agent. In some embodiments, a protein is in a complex with, or is in association with, a nucleic acid, e.g., RNA. In some embodiments, a protein comprises a ligand binding domain. In some embodiments, a protein comprises an active site (e.g., site of biological or enzymatic activity). In some embodiments, a protein comprises an allosteric site (e.g., site of a protein that can bind to a ligand that can be remote from an active site). Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning. A Laboratory Manual [4^{th} ed., Cold Spring Harbor Laboratory Press, Cold Spring Harbor, N.Y. (2012)], the entire contents of which are incorporated herein by reference.

A “variant” of a particular polypeptide or polynucleotide has one or more additions, substitutions, and/or deletions with respect to the polypeptide or polynucleotide, which may be referred to as the “original polypeptide” or “original polynucleotide,” respectively. An addition may be an insertion or may be at either terminus. A variant may be shorter or longer than the original polypeptide or polynucleotide. The term “variant” encompasses “fragments”. A “fragment” is a continuous portion of a polypeptide or polynucleotide that is shorter than the original polypeptide. In some embodiments a variant comprises or consists of a fragment. In some embodiments a fragment or variant is at least 20%, 30%, 40%, 50%, 60%, 70%, 80%, 90%, 95%, 96%, 97%, 98%, 99%, or more as long as the original polypeptide or polynucleotide. A fragment may be an N-terminal, C-terminal, or internal fragment. In some embodiments a variant polypeptide comprises or consists of at least one domain of an original polypeptide. In some embodiments a variant polypeptide or polynucleotide comprises or consists of a polypeptide or polynucleotide that is at least 70%, 80%, 90%, 95%, 96%, 97%, 98%, 99%, or more identical in sequence to the original polypeptide or polynucleotide. In some embodiments a variant polypeptide or polynucleotide comprises or consists of a polypeptide or polynucleotide that is over at least 50%, 60%, 70%, 80%, 90%, 95%, 96%, 97%, 98%, 99%, or 100% of the original polypeptide or polynucleotide. In some embodiments the sequence of a variant polypeptide comprises or consists of a sequence that has N amino acid differences with respect to an original sequence, wherein N is any integer up to 1%, 2%, 5%, or 10% of the number of amino acids in the original polypeptide, where an “amino acid difference” refers to a substitution, insertion, or deletion of an amino acid. In some embodiments a substitution is a conservative substitution. Conservative substitutions may be made, e.g., on the basis of similarity in side chain size, polarity, charge, solubility, hydrophobicity, hydrophilicity and/or the amphipathic nature of the residues involved. In some embodiments, conservative substitutions may be made according to Table A, wherein amino acids in the same block in the second column and in the same line in the third column may be substituted for one another other in a conservative substitution. Certain conservative substitutions are substituting an amino acid in one row of the third column corresponding to a block in the second column with an amino acid from another row of the third column within the same block in the second column.

TABLE A

| | | |
|---|---|---|
| Aliphatic | Non-polar | G A P |
| | | I L V |
| | Polar (uncharged) | C S T M |
| | | N Q |
| | Polar (charged) | D E |
| | | K R |
| Aromatic | | H F Y W |

In some embodiments, proline (P), cysteine (C), or both are each considered to be in an individual group. Within a particular group, certain substitutions may be of particular interest in certain embodiments, e.g., replacements of leucine by isoleucine (or vice versa), serine by threonine (or vice versa), or alanine by glycine (or vice versa).

In some embodiments a variant is a biologically active variant, i.e., the variant at least in part retains at least one activity of the original polypeptide or polynucleotide. In some embodiments a variant at least in part retains more than one or substantially all known biologically significant activities of the original polypeptide or polynucleotide. An activity may be, e.g., a catalytic activity, binding activity, ability to perform or participate in a biological structure or process, etc. In some embodiments an activity of a variant may be at least 10%, 20%, 30%, 40%, 50%, 60%, 70%, 80%, 90%, 95%, or more, of the activity of the original polypeptide or polynucleotide, up to approximately 100%, approximately 125%, or approximately 150% of the activity of the original polypeptide or polynucleotide, in various embodiments. In some embodiments a variant, e.g., a biologically active variant, comprises or consists of a polypeptide at least 95%, 96%, 97%, 98%, 99%, 99.5% or 100% identical to an original polypeptide or over at least 70%, 75%, 80%, 85%, 90%, 95%, 96%, 97%, 98%, or 99% or 100% of the original polypeptide. In some embodiments an alteration, e.g., a substitution or deletion, e.g., in a functional variant, does not alter or delete an amino acid or nucleotide that is known or predicted to be important for an activity, e.g., a known or predicted catalytic residue or residue involved in binding a substrate or cofactor. Variants may be tested in one or more suitable assays to assess activity.

An “antibody” (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. Antibodies may be produced from a variety of hosts, including mammalian (e.g., mouse, rabbit, etc) cells. As described herein, a mouse monoclonal anti-HA (hemagglutinin) antibody may be used for detection of binding of a compound of interest to an androgen receptor.

“Prostate specific antigen” or “PSA” is a glycoprotein enzyme encoded by the KLK3 gene. PSA is also known as gamma-seminoprotein or kallikrein-3 (KLK3). PSA is generally used as an indication of prostate health, wherein PSA is present in small quantities in the serum of men with healthy (e.g., normal) prostates and elevated PSA levels often indicate the presence of prostate cancer or other prostate disorders. PSA may also indicate the presence of prostatitis or benign prostatic hyperplasia.

As used herein, the term “purified” refers to agents that have been separated from most of the components with which they are associated in nature or when originally generated or with which they were associated prior to purification. In general, such purification involves action of the hand of man. Purified agents may be partially purified, substantially purified, or pure. Such agents may be, for example, at least 50%, 60%, 70%, 75%, 80%, 85%, 90%, 95%, 96%, 97%, 98%, 99%, or more than 99% pure. In some embodiments, a nucleic acid, polypeptide, or small molecule is purified such that it constitutes at least 75%, 80%, 85%, 90%, 95%, 96%, 97%, 98%, 99%, or more, of the total nucleic acid, polypeptide, or small molecule material, respectively, present in a preparation. In some embodiments, an organic substance, e.g., a nucleic acid, polypeptide, or small molecule, is purified such that it constitutes at least 75%, 80%, 85%, 90%, 95%, 96%, 97%, 98%, 99%, or more, of the total organic material present in a preparation. Purity may be based on, e.g., dry weight, size of peaks on a chromatography tracing (GC, HPLC, etc.), molecular abundance, electrophoretic methods, intensity of bands on a gel, spectroscopic data (e.g., NMR), elemental analysis, high throughput sequencing, mass spectrometry, or any art-accepted quantification method. In some embodiments, water, buffer substances, ions, and/or small molecules (e.g., synthetic precursors such as nucleotides or amino acids), can optionally be present in a purified preparation. A purified agent may be prepared by separating it from other substances (e.g., other cellular materials), or by producing it in such a manner to achieve a desired degree of purity. In some embodiments “partially purified” with respect to a molecule produced by a cell means that a molecule produced by a cell is no longer present within the cell, e.g., the cell has been lysed and, optionally, at least some of the cellular material (e.g., cell wall, cell membrane(s), cell organelle(s)) has been removed and/or the molecule has been separated or segregated from at least some molecules of the same type (protein, RNA, DNA, etc.) that were present in the lysate.

The term “sample” may be used to generally refer to an amount or portion of something. A sample may be a smaller quantity taken from a larger amount or entity; however, a complete specimen may also be referred to as a sample where appropriate. A sample is often intended to be similar to and representative of a larger amount of the entity of which it is a sample. In some embodiments a sample is a quantity of a substance that is or has been or is to be provided for assessment (e.g., testing, analysis, measurement) or use. A sample may be any biological specimen. In some embodiments, a sample is a cell lysate (e.g., a fluid comprising the contents of lysed cells). In some embodiments a sample comprises a body fluid such as blood, cerebrospinal fluid, (CSF), sputum, lymph, mucus, saliva, a glandular secretion, or urine. In some embodiments a sample comprises cells, tissue, or cellular material (e.g., material derived from cells, such as a cell lysate or fraction thereof). A sample may be obtained from (i.e., originates from, was initially removed from) a subject. Methods of obtaining biological samples from subjects are known in the art and include, e.g., tissue biopsy, such as excisional biopsy, incisional biopsy, core biopsy; fine needle aspiration biopsy; surgical excision, brushings; lavage; or collecting body fluids that may contain cells, such as blood, sputum, lymph, mucus, saliva, or urine. A sample is often intended to be similar to and representative of a larger amount of the entity of which it is a sample. A sample of a cell line comprises a limited number of cells of that cell line. In some embodiments a sample may be obtained from an individual who has been diagnosed with or is suspected of having a neurodegenerative disease. In some embodiments a sample is obtained from skin or blood. In some embodiments a sample contains at least some intact cells. In some embodiments a sample retains at least some of the microarchitecture of a tissue from which it was removed. A sample may be subjected to one or more processing steps, e.g., after having been obtained from a subject, and/or may be split into one or more portions. The term sample encompasses processed samples, portions of samples, etc., and such samples are, where applicable, considered to have been obtained from the subject from whom the initial sample was removed. A sample may be procured directly from a subject, or indirectly, e.g., by receiving the sample from one or more persons who procured the sample directly from the subject, e.g., by performing a biopsy, surgery, or other procedure on the subject.

The term “small molecule” as used herein, is an organic molecule that is less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

The term “compound” as used herein encompasses any small molecule, peptide, nucleic acid, protein, or derivative thereof that may be used to modulate a target of interest (e.g., a transcription factor). In certain embodiments, a compound identified in the present disclosure is able to modulate the androgen receptor. The term “compound” and “agent” are used interchangeably.

A “subject” may be any vertebrate organism in various embodiments. A subject may be individual to whom an agent is administered, e.g., for experimental, diagnostic, and/or therapeutic purposes or from whom a sample is obtained or on whom a procedure is performed. In some embodiments a subject is a mammal, e.g. a human, non-human primate, or rodent (e.g., mouse, rat, rabbit). In some embodiments, a subject has been diagnosed with prostate cancer.

“Treat,” “treating” and similar terms as used herein in the context of treating a subject refer to providing medical and/or surgical management of a subject. Treatment may include, but is not limited to, administering an age or composition (e.g., a pharmaceutical composition) to a subject. Treatment is typically undertaken in an effort to alter the course of a disease (which term is used to indicate any disease, disorder, syndrome or undesirable condition warranting or potentially warranting therapy) in a manner beneficial to the subject. The effect of treatment may include reversing, alleviating, reducing severity of, delaying the onset of, curing, inhibiting the progression of, and/or reducing the likelihood of occurrence or recurrence of the disease or one or more symptoms or manifestations of the disease. A therapeutic agent may be administered to a subject who has a disease or is at increased risk of developing a disease relative to a member of the general population. In some embodiments a therapeutic agent may be administered to a subject who has had a disease but no longer shows evidence of the disease. The agent may be administered e.g., to reduce the likelihood of recurrence of evident disease. A therapeutic agent may be administered prophylactically, i.e., before development of any symptom or manifestation of a disease. “Prophylactic treatment” refers to providing medical and/or surgical management to a subject who has not developed a disease or does not show evidence of a disease in order, e.g., to reduce the likelihood that the disease will occur or to reduce the severity of the disease should it occur. The subject may have been identified as being at risk of developing the disease (e.g., at increased risk relative to the general population or as having a risk factor that increases the likelihood of developing the disease.

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Anti-cancer agents encompass biotherapeutic anti-cancer agents as well as chemotherapeutic agents. Exemplary biotherapeutic anti-cancer agents include, but are not limited to, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g. HERCEPTIN (trastuzumab), T-DM1, AVASTIN (bevacizumab), ERBITUX (cetuximab), VECTIBIX (panitumumab), RITUXAN (rituximab), BEXXAR (tositumomab)).

Exemplary chemotherapeutic agents include, but are not limited to, anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF(2)341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI(2)7 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

The terms “agent” and “therapeutic agent” are used herein to refer to any substance, compound (e.g., molecule), supramolecular complex, material, or combination or mixture thereof. A compound may be any agent that can be represented by a chemical formula, chemical structure, or sequence. Example of agents, include, e.g., small molecules, polypeptides, nucleic acids (e.g., RNAi agents, antisense oligonucleotide, aptamers), lipids, polysaccharides, etc. In general, agents may be obtained using any suitable method known in the art. The ordinary skilled artisan will select an appropriate method based, e.g., on the nature of the agent. An agent may be at least partly purified. In some embodiments an agent may be provided as part of a composition, which may contain, e.g., a counter-ion, aqueous or non-aqueous diluent or carrier, buffer, preservative, or other ingredient, in addition to the agent, in various embodiments. In some embodiments an agent may be provided as a salt, ester, hydrate, or solvate. In some embodiments an agent is cell-permeable, e.g., within the range of typical agents that are taken up by cells and acts intracellularly, e.g., within mammalian cells, to produce a biological effect. Certain compounds may exist in particular geometric or stereoisomeric forms. Such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof are encompassed by this disclosure in various embodiments unless otherwise indicated. Certain compounds may exist in a variety or protonation states, may have a variety of configurations, may exist as solvates [e.g., with water (i.e. hydrates) or common solvents] and/or may have different crystalline forms (e.g., polymorphs) or different tautomeric forms. Embodiments exhibiting such alternative protonation states, configurations, solvates, and forms are encompassed by the present disclosure where applicable.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, hematological malignancies. Additional exemplary cancers include, but are not limited to, lung cancer (e.g., bronchogenic carcinoma, small cell lung cancer (SCLC), non-small cell lung cancer (NSCLC), adenocarcinoma of the lung); kidney cancer (e.g., nephroblastoma, a.k.a. Wilms' tumor, renal cell carcinoma); acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); heavy chain disease (e.g., alpha chain disease, gamma chain disease, mu chain disease; hemangioblastoma; hypopharynx cancer; inflammatory myofibroblastic tumors; immunocytic amyloidosis; liver cancer (e.g., hepatocellular cancer (HCC), malignant hepatoma); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma, metastatic prostate cancer, castration-resistant prostate cancer); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

### BRIEF DESCRIPTION OF THE DRAWINGS

The accompanying drawings, which constitute a part of this specification, illustrate several exemplary embodiments of the invention and together with the description, serve to explain certain principles of the invention. The embodiments disclosed in the drawings are exemplary and do not limit the scope of this disclosure.

FIG. 1 shows an overview of a small molecule microarray (SMM) screening, which revealed potential binders of AR-v7 from a plurality of compounds.

FIG. 2 shows the results of a qPCR assay in LNCaP cells, wherein PSA was used as a readout for both AR-v7 and AR-FL activity. Selected SMM assay positives were administered to cells and PSA expression was evaluated.

FIG. 3 shows qPCR actives evaluated in an dual luciferase reporter assay, wherein 85 prioritized compounds from the qPCR assay were further evaluated using a stable LNCaP cell line with AR-v7 under doxorubicin (DOX) control and MMTV-driven firefly luciferase. CMV-driven Renilla luciferase is also included as a control. The relative luciferase activity is the firefly luciferase activity normalized to the Renilla luciferase activity.

FIGS. 4A to 4D show a summary of results for KI-ARv-01 in a Reporter Assay at 24 hours (FIG. 4A), Reporter Assay and CTG assay at 24 hours (FIG. 4B), CTG viability assay at −3 days (FIG. 4C), and CTG viability assay at −5 days (FIG. 4D). The chemical name for KI-ARv-01 is N-{2-[(1S*,2S*,4S*)-bicyclo[2.2.1]hept-5-en-2-yl]ethyl}-3-{[1-(cyclopropylcarbonyl)-4-piperidinyl]oxy}benzamide, and it has the following identification numbers: CBL_ID: PW2571, ChemBridge ID: 37593804, and PubChem ID: 25476891. KI-ARv-01 has a C Log P of 3.27, and a molecular weight of 408.55 g/mol. The IC₅₀ is 5.43 μM.

FIG. 5 shows the results of an assay for KI-ARv-02. The chemical name for KI-ARv-02 is 4-((((1R,4R)-bicyclo[2.2.1]hept-5-en-2-yl)methyl)amino)-N,5-dimethyl-N-(quinolin-5-ylmethyl)thieno[2,3-d]pyrimidine-6-carboxamide, and it has the following identification numbers: ChemBridge ID: 49307780 and PubChem ID: 26358645. KI-ARv-02 has a C Log P of 5.48, and a molecular weight of 496.61 g/mol. The IC₅₀ is 6.86 μM.

FIGS. 6A to 6D show a summary of results for KI-ARv-03 in a Reporter Assay at 24 hours (FIG. 6A), Reporter Assay and CTG assay at 24 hours (FIG. 6B), CTG viability assay at −3 days (FIG. 6C), and CTG viability assay at −5 days (FIG. 6D). The chemical name for KI-ARv-03 is (1R*,3R*)—N-(5-propylpyrazolo[1,5-a]pyrimidin-7-yl)-1,3-cyclopentanediamine, and it has the following identification numbers: CBL_ID: GB 1987, ChemBridge ID: 94082098, and PubChem ID:56915928. KI-ARv-03 has a C Log P of 2.54, and a molecular weight of 259.36 g/mol. The IC₅₀ is 7.6 μM.

FIGS. 7A to 7D show a summary of results for KI-ARv-04 in a Reporter Assay at 24 hours (FIG. 7A), Reporter Assay and CTG assay at 24 hours (FIG. 7B), CTG viability assay at −3 days (FIG. 7C), and CTG viability assay at −5 days (FIG. 7D). The chemical name for KI-ARv-04 is (1R*,5S*,6r)-6-{[4-(4-fluorophenyl)-1,4-diazepan-1-yl]methyl}-3-azabicyclo[3.1.0]hexane, and it has the following identification numbers: CBL_ID: GB 1458, ChemBridge ID: 23460329, and PubChem ID: 56886853. KI-ARv-04 has a C Log P of 1.74, and a molecular weight of 289.4 g/mol. The IC₅₀ is 11.29 μM.

FIGS. 8A and 8B show a summary of results for KI-ARv-05 in a Reporter Assay at 24 hours (FIG. 8A) and Reporter Assay and CTG assay at 24 hours (FIG. 8B). The chemical name for KI-ARv-05 is 2-amino-6-(2,5-dimethyl-3-thienyl)-4-[2-(ethylamino)pyrimidin-5-yl]nicotinonitrile and it has the following identification numbers: CBL_ID: OR0124, ChemBridge ID: 11815275, and PubChem ID: 56738557. KI-ARv-05 has a C Log P of 3.4, and a molecular weight of 350.45 g/mol. The IC₅₀ is 50 μM.

FIGS. 9A to 9D show a summary of results for KI-ARv-06 in a Reporter Assay at 24 hours (FIG. 9A), Reporter Assay and CTG assay at 24 hours (FIG. 9B), CTG viability assay at −3 days (FIG. 9C), and CTG viability assay at −5 days (FIG. 9D). The chemical name for KI-ARv-06 is 4-methyl-6-{4-[(3-pyrrolidin-1-ylbenzyl)amino]piperidin-1-yl}pyrimidin-2-amine and it has the following identification numbers: CBL_ID: PI1178, ChemBridge ID: 90540306, and PubChem ID: 72930383. KI-ARv-06 has a C Log P of 2.22, and a molecular weight of 366.51 g/mol. The IC₅₀ is 14.8 μM.

FIGS. 10A to 10D show a summary of results for KI-ARv-07 in a Reporter Assay at 24 hours (FIG. 10A), Reporter Assay and CTG assay at 24 hours (FIG. 10B), CTG viability assay at −3 days (FIG. 10C), and CTG viability assay at −5 days (FIG. 10D). The chemical name for KI-ARv-07 is 3-((1-(cyclopropanecarbonyl)piperidin-4-yl)oxy)-N-(((1R,4R)-spiro[bicyclo[2.2.1]heptane-7,1′-cyclopropan]-2-en-5-yl)methyl)benzamide. KI-ARv-07 has the following identification numbers: CBL_ID: PW2784, ChemBridge ID: 42092631, and PubChem ID: 45208568. KI-ARv-07 has a C Log P of 3.58, and the IC₅₀ is >50 μM.

FIGS. 11A to 11D show a summary of results for KI-ARv-08 in a Reporter Assay at 24 hours (FIG. 11A), Reporter Assay and CTG assay at 24 hours (FIG. 11B), CTG viability assay at −3 days (FIG. 11C), and CTG viability assay at −5 days (FIG. 11D). The chemical name for KI-ARv-08 is N-{2-[(1S*,2S*,4S*)-bicyclo[2.2.1]hept-5-en-2-yl]ethyl}-3-{[1-(methylsulfonyl)-4-piperidinyl]oxy}benzamide and it has the following identification numbers: CBL_ID: PW2852, ChemBridge ID: 44256638, and PubChem ID: 25297857. KI-ARv-08 has a C Log P of 3.29 and a molecular weight of 418.56 g/mol. The IC₅₀ is 24.66 μM.

FIGS. 12A to 12D show a summary of results for KI-ARv-09 in a Reporter Assay at 24 hours (FIG. 12A), Reporter Assay and CTG assay at 24 hours (FIG. 12B), CTG viability assay at −3 days (FIG. 12C), and CTG viability assay at −5 days (FIG. 12D). The chemical name for KI-ARv-09 is N-[(1R*,2S*,4R*)-bicyclo[2.2.1]hept-5-en-2-ylmethyl]-6-{[4-(4-methoxyphenyl)-1-piperazinyl]carbonyl}-5-methylthieno[2,3-d]pyrimidin-4-amine and it has the following identification numbers: CBL_ID: RO3585, ChemBridge ID: 13542678, and PubChem ID: 42407573. KI-ARv-09 has a C Log P of 5.58, and a molecular weight of 489.64 g/mol. The IC₅₀ is >50 μM.

FIGS. 13A and 13B show a summary of results for an alternative compound, RO3682, in a Reporter Assay at 24 hours (FIG. 13A) and Reporter Assay and CTG assay at 24 hours (FIG. 13B), The chemical name for RO3682 is 4-{[(1R*,2S*,4R*)-bicyclo[2.2.1]hept-5-en-2-ylmethyl]amino}-5-methyl-N-(4-pyridinylmethyl)thieno[2,3-d]pyrimidine-6-carboxamide and it has the following identification numbers: CBL_ID: RO3682, ChemBridge ID: 57100162, and PubChem ID: 42433246. RO3682 has a C Log P of 4.19, and a molecular weight of 405.53 g/mol. The IC₅₀ is 12.5 μM.

FIG. 14 shows the results of an assay for an alternative compound, OR0392, which has the following identification numbers: CBL_ID: OR0392, ChemBridge ID: 89868118, and PubChem ID: 56914190. OR0392 has a C Log P of 3.28, and a molecular weight of 319.36 g/mol.

### DETAILED DESCRIPTION OF CERTAIN EMBODIMENTS

### Screening Method for Identifying Androgen Receptor Modulators

The screening methods disclosed herein take advantage of screening an androgen receptor variant (e.g., AR-v7) that is resistant to traditional therapies due to its lack of a ligand binding domain (LBD). Traditional therapies such as enzalutimde target the LBD of full-length androgen receptor (AR-FL). C-terminal variations in the androgen receptor (e.g., lack of a ligand binding domain) promote a constitively active androgen receptor variant (AR-v) that effectively mimics AR-FL in its ability to transactive androgen receptor elements (AREs) without androgen stimulation. Thus, compositions described herein may be used to treat castration-resistant prostate cancer or prostate cancer that has relapsed after being treated with traditional therapies, indications for which there are currently no available treatments. Screening the androgen receptor variant using the methods disclosed herein takes advantage of crucial differences in structure between AR-vs and AR-FL. Because most AR-vs lack a ligand binding domain (LBD), the fact that traditional therapies tend to target the ligand binding domain to inhibit the androgen receptor render all currently available therapies ineffective against AR-vs. The screening method disclosed herein has identified compounds that modulate the androgen receptor or fragments thereof, including the clinically relevant variant AR-v7.

In one aspect, the screening methods disclosed herein for identifying an androgen receptor modulator may involve the use of a small molecule microarray (SMM) screen. Microarray screening is a high-throughput method for interrogating a plurality of compounds for their ability to bind to a variety of targets of interest. In general, the compounds of the microarray are immobilized onto a solid surface (e.g., glass or plastic) via a number of covalent and non-covalent technologies. Substrate technologies and immobilization chemistries for the generation of microarrays (e.g., small molecule microarrays) are known in the art. See, e.g., US 2009/0221433; Uttamchandani, M., Walsh, D. P., Yao, S. Q., and Chang, Y. (2005) Small molecule microarrays: recent advances and applications. Curr Opin Chem Bio, 9: 4-13. A microarray may be an oligonucleotide (e.g., DNA or RNA) microarray, a peptide (e.g., cyclic peptide, macrocyclic peptide, synthetic peptide) microarray, or a small molecule microarray, for example. In certain embodiments, the microarray is a small molecule microarray comprising a plurality of chemically diverse compounds. The small molecule microarray may be comprised of synthetic compounds, naturally occurring compounds, or a combination thereof. A small molecule microarray may be a collection of compounds with particular protein-binding behaviors. The compounds in the small molecule microarray may have been selected for specific structural (e.g., chemical moiety, stereochemistry, etc.) or chemicophysical properties (e.g., solubility). Small molecule microarrays may comprise any number of compounds to be screened against a target of interest. For example, a small molecule microarray may comprise approximately 50,000 compounds, or as little as a few hundred compounds, depending on the purpose of screening the small molecule microarray against a target of interest.

In general, a SMM screen involves exposing the small molecule array to (e.g., contacting the small molecule microarray with) an agent of interest (e.g., a protein). Microarrays have been used to identify compounds that target and inhibit the human papillomavirus and the ETV1 transcription factor oncoprotein [US 2005/0123902; Pop M S, et al (2014) Mol Cancer Ther, 13(6).]. In certain embodiments, a small molecule microarray is exposed to (e.g., contacted with) a protein. The protein used in the SMM screen may be purified. In certain embodiments, the protein used in the SMM screen is a full-length androgen receptor (AR-FL), or variant thereof. In certain embodiments, the protein used in the SMM screen is an androgen receptor variant (AR-v). In certain embodiments, the protein used in the SMM screen is the androgen receptor variant AR-v7, or variant thereof.

A SMM is also compatible with complex mixtures and cell lysates comprising endogenous proteins, allowing the user to bypass the purification process. The cell lysate can be obtained from any cell line obtained from any organism that is suitable for protein expression, including mammalian cell lines (e.g., cancer cell lines), yeast cell lines, and bacterial cell lines, for example. In certain embodiments, a cell lysate is obtained from a prostate cancer cell line. The cell lysate may be obtained from cells overexpressing an androgen receptor, or variant thereof, such as LNCaP cells. In certain embodiments, a cell lysate is obtained from a castration-resistant prostate cancer cell line. Human cancer cell lines that have been generated (e.g., derived) from subjects with prostate cancer (e.g., human tumors) are known in the art [Russell, P. J. and Kingsley, E. A. (eds.) (2003) Prostate cancer methods and protocols, pp. 21-39].

In SMM screens involving purified proteins and cell lysates, antibodies are used against the protein target, or overexpressed, epitope-tagged proteins. Detection of a protein-small molecule interaction on a microarray is carried out by exposing the small molecule microarray to (e.g., contacting the small molecule microarray with) a fluorescently labeled antibody against the protein interest or an epitope tag. Common epitope tags are FLAG, HA, His, Myc, V5, Xpress, Thrombin, BAD (biotin acceptor domain), Factor Xa, VSVG, SV40 NLS, Protein C, S Tag, OneStrap, and SB1, among others. In certain embodiments, the epitope is HA (hemagglutinin). In certain embodiments, the HA tag comprises the amino acid sequence YPYDVPDYA (SEQ ID NO: 3). The epitope tag may be associated covalently or non-covalently with the protein target of interest in any orientation (e.g., attached to the C-terminus, N-terminus, or intervening sequence of the protein target of interest). The epitope tag may be attached to the protein target of interest with a linker of any length or composition necessary to achieve the appropriate binding interaction with the antibody used to detect the epitope. In certain embodiments, the epitope tag is attached to an androgen receptor or fragment thereof. In certain embodiments, HA is attached to an androgen receptor or fragment thereof, i. e., the androgen receptor or fragment thereof is HA-tagged. For detection of a protein-small molecule interaction, the SMM can be incubated with a solution comprising the appropriate antibody against the protein of interest or the epitope tag. Antibodies suitable for biochemical applications such as ELISA, immunoblotting, immunocytochemistry, immunoprecipitation, and the like are well known in the art. See, e.g., Harlow, E. and Lane, D., Antibodies—A Laboratory Manual, Cold Spring Harbor Laboratory Press, Cold Spring Harbor, 1988. For example, for the detection of HA-tagged proteins, anti-HA antibody recognizes native as well as denatured or reduced forms of HA-tagged proteins, and is reactive with N- or C-terminal HA-tagged fusion proteins. In certain embodiments, an anti-HA antibody is used to detect the HA-tagged androgen receptor or fragment thereof. In certain embodiments, the anti-HA antibody is a mouse monoclonal anti-HA antibody. To promote detection of the antibody, the antibody will generally include a fluorescent label (e.g., Fluor-labeled antibody). Briefly, labels (e.g., a fluorophore) are frequently conjugated to the primary amine (e.g., lysine) moiety (—NH) on the solvent-accessible surface of the antibody using a variety of conjugation techniques, including glutaraldehyde and reductive amination crosslinking approaches (e.g., using horseradish peroxidase (HRP) or alkaline phosphatase (AP)). Labels can also be attached to the sulfahydryl (—SH) groups of cysteine residues or to reactive aldehydes (—CHO) present on the carbohydrate moiety of a glycosylated antibody using the appropriate conjugation chemistry, which would be apparent to a skilled artisan. In certain embodiments, the antibody is Cy5-labeled. In certain embodiments, Cy5-labeled anti-HA mouse monoclonal antibodies are used to detect the binding of the HA-tagged androgen receptor or fragment thereof to a compound in the SMM, wherein the fluorescence-based readout is provided by a standard fluorescent scanner with plate-reading capability (e.g., GenePix 4000B fluorescent scanner). A compound that binds to the protein target of interest, which can then be detected by the presence of a fluorescence signal (e.g., from the fluor-labeled antibody associated with the protein target of interest) is said to be an “assay positive” compound.

In some examples, the antibody disclosed herein specifically binds a target antigen, such as androgen receptor or a fragment thereof. In certain embodiments, the antibody disclosed herein specifically binds AR-v7. An antibody that “specifically binds” (used interchangeably herein) to a target or an epitope is a term well understood in the art, and methods to determine such specific binding are also well known in the art. A molecule is said to exhibit “specific binding” if it reacts or associates more frequently, more rapidly, with greater duration and/or with greater affinity with a particular target antigen than it does with alternative targets. An antibody “specifically binds” to a target antigen if it binds with greater affinity, avidity, more readily, and/or with greater duration than it binds to other substances. For example, an antibody that specifically (or preferentially) binds to a HA epitope is an antibody that binds this HA epitope with greater affinity, avidity, more readily, and/or with greater duration than it binds to other epitopes or non-HA epitopes. It is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, but not necessarily, reference to binding means preferential binding.

In general, a Z score for every compound (e.g., small molecule) in the SMM is calculated, taking into account the presence of promiscuous binders (e.g., compounds that bind non-specifically to a protein or multiple proteins). The terms “Z score” and “composite Z score” as used herein may be used interchangeably, unless otherwise noted. ChemBank contains composite Z scores for SMMs available to all screeners the Broad Institute (www.chembank.broad.harvard.edu/). In certain embodiments, a Z score is assigned to each compound in the small molecule microarray. In certain embodiments, a Z score is assigned to each compound in the small molecule microarray comprising compounds that have been shown to be efficacious in prostate cancer cell lines. The Z score is a term understood in the art. Briefly, the Z score indicates how many standard deviations an element is from the mean (e.g. a Z score of 0 indicates an element is equal to the mean, a Z score of 1 indicates an element is 1 standard deviation greater than the mean, a Z score of −1 indicates that an element is 1 standard deviation less than the mean, and so on). A skilled artisan will appreciate that the Z score can be used to determine compounds that may preferentially bind to the target protein of interest, i.e., a compound with a higher Z score may more preferentially bind to the target protein of interest than a compound with a lower Z score. The threshold Z score for selecting compounds that may preferentially bind to an androgen receptor, or variant thereof, will vary depending on the androgen receptor, or variant thereof, being assayed, the compounds comprising the SMM, assay conditions (e.g., incubation times, suitable buffer conditions, etc.), and such. Optimizing the SMM screen as disclosed herein to identify one or more compounds that bind to a protein target of interest is within the ability of a person of skill in the art.

In certain embodiments, one or more compounds is selected from a SMM screen when a compound is assay positive. In certain embodiments, one or more compounds is selected from a SMM screen when a compound is (i) assay positive, and (ii) the compound has a Z score of at least 1.5. In certain embodiments, one or more compounds is selected from a SMM screen when a compound is (i) assay positive, and (ii) the compound has a Z score of at least 2.0. In certain embodiments, one or more compounds is selected from a SMM screen when a compound is (i) assay positive, and (ii) the compound has a Z score of at least 2.5. In certain embodiments, one or more compounds is selected from a SMM screen when a compound is (i) assay positive, and (ii) the compound has a Z score of at least 3.0. In certain embodiments, one or more compounds is selected from a SMM screen when a compound is (i) assay positive, and (ii) the compound has a Z score of at least 3.5. In certain embodiments, one or more compounds is selected from a SMM screen when a compound is (i) assay positive, and (ii) the compound has a Z score of at least 4.0. In certain embodiments, one or more compounds is selected from a SMM screen when a compound is (i) assay positive, and (ii) the compound has a Z score of at least 4.5. In certain embodiments, one or more compounds is selected from a SMM screen when a compound is (i) assay positive, and (ii) the compound has a Z score of at least 5.0. In certain embodiments, one or more compounds is selected from a SMM screen when a compound is (i) assay positive, and (ii) the compound has a Z score of at least 5.5. In certain embodiments, one or more compounds is selected from a SMM screen when a compound is (i) assay positive and (ii) the compound has a Z score of at least 6.0. In certain embodiments, one or more compounds is selected from a SMM screen when a compound is (i) assay positive, and (ii) the compound has a Z score of at least 6.5. In certain embodiments, one or more compounds is selected from a SMM screen when a compound is (i) assay positive, and (ii) the compound has a Z score of at least 7.0. In certain embodiments, one or more compounds is selected from a SMM screen when a compound is (i) assay positive, and (ii) the compound has a Z score of at least 7.5. In certain embodiments, one or more compounds is selected from a SMM screen when a compound is (i) assay positive, and (ii) the compound has a Z score of at least 8.0.

In certain embodiments, one or more compounds that are selected in the SMM screen can be further evaluated by one or more secondary binding assays (e.g., surface plasmon resonance (SPR), thermal shift, and calorimetry), functional assays, or phenotypic assays (e.g., qPCR). Secondary binding assays may be used to determine whether or not the compound interacts directly with the target protein of interest (e.g., binds to the target protein) or to another protein in a complex (e.g., a member other than the target protein of interest in a multimeric complex comprising the target protein of interest), or to measure the binding affinities.

In certain embodiments, one or more compounds selected from the SMM screen are further selected using one or more additional screens. In certain embodiments, one or more compounds selected from the SMM screen are screened using a phenotypic assay. In certain embodiments, one or more compounds selected from the SMM screen are submitted to a screen, wherein the screen comprises a phenotypic assay involving qPCR. The term “qPCR” refers to a quantitative polymerase chain reaction or a real-time polymerase chain reaction. qPCR techniques for amplifying and detecting changes in concentration of a specific DNA or RNA sequence (e.g., amplicon) are well known in the art. A qPCR assay is used quantitatively or semi-quantitatively (e.g., above or below a certain amount of DNA molecules) to measure gene expression in a cell [Dhanasekaran, S. et al. (2010) Immunol Methods, 354, 34-39]. In certain embodiments, a qPCR assay is used quantitatively or semi-quantitatively (e.g., above or below a certain amount of DNA molecules) to measure prostate-specific antigen (PSA) gene expression in a cell, wherein the cell is a LNCaP cell. LNCaP cells are a cell line that are androgen-sensitive human prostate adenocarcinoma cells. In certain embodiments, a qPCR assay is used quantitatively or semi-quantitatively (e.g., above or below a certain amount of DNA molecules) to measure PSA gene expression in a LNCaP cell, wherein the LNCaP cell expresses an androgen receptor, or variant thereof. The qPCR assay can be used to quantitate DNA expression levels in a cell, often with the use of fluorescent DNA-binding dyes or special probes that comprise a fluorophore attached to one end and a quencher molecule attached to the opposite end. Normally, the fluorophore is covalently attached to the 5′-end of the oligonucleotide probe and the quencher is attached to the 3′-end of the oligonucleotide probe. The oligonucleotide probe comprises a nucleotide sequence that is complementary to the gene of interest (e.g., gene whose expression level is to be measured) that can hybridize (e.g., associate) to the DNA sequence comprising the gene of interest. One example of a probe that is routinely used in the art is the TaqMan™ probe. The TaqMan™ probe has 5′-to-3′ exonuclease activity and a donor fluorophore and quencher attached to the 5′-end and 3′-end, respectively. Probes of this type are routinely used in the art. In certain embodiments, a TaqMan™ probe is used to measure the expression level of a gene of interest. In an embodiment, a TaqMan™ probe is used to measure the expression level of prostate specific antigen (PSA). As described herein, the PSA expression level may be used as a readout for the activity level of an androgen receptor or fragment thereof. In certain embodiments, one or more compounds selected from the SMM screen are submitted to a qPCR assay. In certain embodiments, one or more compounds selected from the SMM screen are submitted to a qPCR assay, wherein the qPCR assay readout is the PSA expression level in a cell. In certain embodiments, a compound submitted to the qPCR assay leads to a decrease in the PSA expression level in a cell relative to a cell that has not been treated (e.g., untreated, control cell). In certain embodiments, a compound submitted to the qPCR assay leads to an increase in the PSA expression level in a cell relative to a cell that has not been treated (e.g., untreated, control cell). In certain embodiments, an increase in the PSA expression level of a cell exposed to (e.g., contacted with) a compound relative to the control may be correlated with an increase in androgen receptor (e.g., AR-FL, AR-v7) activity. In certain embodiments, an increase in the PSA expression level of a cell exposed to (e.g., contacted with) a compound relative to the control may be correlated with an increase in androgen receptor (e.g., AR-FL, AR-v7) activity, that is, the compound is an androgen receptor modulator. In certain embodiments, a decrease in the PSA expression level of a cell exposed to (e.g., contacted with) a compound relative to the control may be correlated with an decrease in androgen receptor (e.g., AR-FL, AR-v7) activity. In certain embodiments, a decrease in the PSA expression level of a cell exposed to (e.g., contacted with) a compound relative to the control may be correlated with an decrease in androgen receptor (e.g., AR-FL, AR-v7) activity, that is, the compound is an androgen receptor modulator. In certain embodiments, a decrease in the PSA expression level of a cell exposed to (e.g., contacted with) a compound relative to the control may be correlated with an decrease in androgen receptor (e.g., AR-FL, AR-v7) activity, wherein the compound is an androgen receptor modulator, and wherein the decrease (e.g., reduction) in PSA expression level is at least 20% in cells exposed to (e.g., contacted with) the androgen receptor modulator compared to the PSA expression level of untreated (e.g., control) cells. In certain embodiments, the decrease (e.g., reduction) in the PSA expression level of cells exposed to (e.g., contacted with) the androgen receptor modulator compared to the PSA expression level of untreated (e.g., control) cells is at least 10%, 20%, 30%, 40%, 50%, 60%, 70%, 80%, 90%, or 100%. In certain embodiments, a decrease in the PSA expression level of a cell exposed to (e.g., contacted with) a compound relative to the control may be correlated with an decrease in androgen receptor (e.g., AR-FL, AR-v7) activity. That is, the compound may be an inhibitor or an antagonist of the androgen receptor, or variant thereof.

In an embodiment, one or more compounds selected from the SMM screen are submitted to a phenotypic assay involving RT-PCR. The term “RT-PCR” refers to reverse-transcription polymerase chain reaction. RT-PCR may be used to qualitatively detect gene expression through creation of complementary DNA (cDNA) transcripts from mRNA. RT-PCR is used to clone expressed genes by reverse transcribing (e.g., transcribing RNA to DNA) the RNA of interest into its DNA complement through the use of the reverse transcriptase enzyme. The cDNA is then amplified by traditional PCR techniques, which are well known in the art. In certain embodiments, a RT-PCR assay is used qualitatively measure gene expression in a cell. The RT-PCR assay may be used to qualitatively measure gene expression in a prostate cancer cell, metastatic prostate cancer cell, a castration-resistant prostate cancer cell, or a cell overexpressing an androgen receptor, or variant thereof. In certain embodiments, a RT-PCR assay is used qualitatively measure gene expression in a cell, wherein the cell is a LNCaP cell. In certain embodiments, a RT-PCR assay is used qualitatively measure gene expression in a cell, wherein the cell is a LNCaP cell and wherein the LNCaP cell is expressing an androgen receptor or fragment thereof.

In certain embodiments, one or more compounds selected from the SMM screen are submitted to a phenotypic assay involving quantitative RT-PCR. The term “quantitative RT-PCR” or “qRT-PCR” refers to a combination of the qPCR and RT-PCR techniques. In certain embodiments, a qRT-PCR assay is used qualitatively to measure gene expression in a cell. The qRT-PCR assay may be used to qualitatively measure gene expression in a prostate cancer cell, metastatic prostate cancer cell, a castration-resistant prostate cancer cell, or a cell overexpressing an androgen receptor, or variant thereof. In certain embodiments, a qRT-PCR assay is used qualitatively to measure gene expression in a LNCaP cell. In certain embodiments, a qRT-PCR assay is used qualitatively to measure gene expression in a cell, wherein the cell is a LNCaP cell, and the LNCaP cell is expressing an androgen receptor or fragment thereof.

In an embodiment, one or more compounds selected from the SMM screen are further screened using a reporter assay. A reporter assay is commonly used to study signaling pathways, gene expression and regulation at the transcriptional level, and the structure of regulatory elements. In general, a reporter assay comprises a regulatory element of interest (e.g. promoter DNA) along with a reporter gene (e.g., gene encoding a reporter protein) cloned into a vector and transfected into cells. In certain embodiments, a reporter is a protein. In certain embodiments, a reporter is a protein with an easily measureable activity. “Easily measureable activity” (or simply “activity”) can refer to any activity that can be measured through methods known in the art, such as fluorescence, chemiluminescence, bioluminescence, enzymatic activities that generate fluorescent or luminescent products, or indirectly with antibodies. The activity of the regulatory element can be directly modulated by experimental conditions (e.g., introduction of a modulator into the cellular environment). Activity is directly correlated to the concentration of reporter produced from the transcription of the reporter gene, i.e., transcription of the promoter leads to production of the reporter, such that strong promoters produce more reporter and weak promoters produce less reporter. In certain embodiments, the reporter protein is luciferase. Luciferase is a generic term for a class of oxidative enzymes that produce bioluminescence, for example firefly luciferase, Renilla luciferase, NanoLuc luciferase, bacterial luciferase, among others. In an embodiment, the regulatory element is a mouse mammary tumor virus (MMTV) promoter, and the MMTV promoter contains one or more androgen response element (ARE). In general, any reporter than contains an ARE can be used in this assay. The MMTV promoter and luciferase are encoded by (e.g., the DNA sequences are contained within) the same vector and transfected into cells, such that activation of the androgen receptor, or variant therof, leads to increased expression of the MMTV-driven luciferase. Design of vectors and transfection of vectors into cells are methods well known in the art. See, e.g., Sambrook, Russell, and Sambrook, Molecular Cloning: A Laboratory Manual, 3^{rd} ed., Cold Spring Harbor Laboratory Press, Cold Spring Harbor, 2001.

A “dual-reporter assay” is conceptually similar to the single reporter assay described above, and can correct for experimental variation (e.g., cell number, transfection efficiency, etc.). In the case of a dual-reporter assay, a “reporter system” is used, where cells are transfected with two plasmids, the first plasmid comprising the regulatory gene of interest and a first reporter, and the second plasmid comprising a constitutive promoter and a second reporter, wherein the second reporter and the first reporter have biologically distinct activities. The ratio of the first reporter activity (e.g., controlled by the regulatory gene of interest) relative to the second reporter activity (e.g., controlled by the constitutive promoter) corrects for experimental variation. For example, a dual-luciferase assay employs two luciferase reporter proteins that each have distinct bioluminescence signatures that can be easily distinguished and measured. In certain embodiments, one or more compounds selected from the SMM screen are submitted to a screen comprising a dual-luciferase assay, the dual-luciferase assay further comprising (i) a first vector comprising a first promoter and a first reporter and (ii) a second vector comprising a second promoter and a second reporter. In certain embodiments, the first vector comprises a first promoter and a first reporter, wherein the first promoter is the MMTV promoter and the first reporter is firefly luciferase. Activation of the androgen receptor, or variant thereof, will promote increased expression of the MMTV-driven firefly luciferase. In certain embodiments, the second vector comprises a second promoter and a second reporter, wherein the second promoter is the cytomegalovirus (CMV) promoter and the second reporter is Renilla luciferase. The CMV promoter is constitutively activated, leading to a basal expression level of Renilla luciferase that serves as a control and can be used to normalize the MMTV-driven firefly luciferase expression level. As described herein, the “relative luciferase activity” is the luciferase activity of a first luciferase as compared to the activity of a second luciferase, wherein expression of the second luciferase is under the control of a constitutively active promoter. In an embodiment, the first luciferase is firefly luciferase. In an embodiment, the second luciferase is Renilla luciferase. In certain embodiments, the Renilla luciferase is under the control of a constitutively active CMV promoter. In certain embodiments, the firefly luciferase is under control of an MMTV promoter that contains one or more androgen response element (ARE).

The screening methods described above may be conducted in any order. For example, compounds selected from the SMM screen may be further screened using a secondary assay involving qPCR or a reporter assay. If the SMM assay is followed by a secondary screen involving qPCR, the compounds selected in the qPCR assay may be further screened with a screen involving a reporter assay. If the SMM assay is followed by a secondary screen involving a reporter assay, the compounds selected in the reporter assay may be further screened with a screen involving qPCR. In certain embodiments, the SMM screen is followed by a secondary screen involving qPCR, which is in turn followed by a screen involving a reporter assay.

In one aspect, the present disclosure provides methods of identifying an androgen receptor modulator, the methods comprising: (a) exposing a plurality of compounds to an androgen receptor, or variant thereof; (b) selecting one or more compounds that bind to the androgen receptor, or variant thereof; (c) exposing the one or more selected compounds that bind the androgen receptor, or variant thereof, individually to cells expressing an androgen receptor, or variant thereof; and (d) selecting one or more compounds that reduce prostate-specific antigen (PSA) expression in the cells. In certain embodiments, the plurality of compounds is provided in a small molecule microarray (SMM). The small molecule microarray may be comprised of approximately 1,000 to approximately 50,000 compounds to be screened against the androgen receptor, or variant thereof. In certain embodiments, the small molecule microarray may be comprised of approximately 50,000 compounds to be screened against the androgen receptor, or variant thereof. In certain embodiments, one or more compounds having an affinity of at least 1 mM for the androgen receptor (e.g., AR-FL, AR-v7), or variant thereof, are selected in step (b) and/or (d). In certain embodiments, one or more compounds having an affinity of approximately 5 nM to approximately 1 mM for the androgen receptor (e.g., AR-FL, AR-v7), or variant thereof, are selected in step (b) and/or (d). In certain embodiments, one or more compounds having an affinity of approximately 1 μM to approximately 1 mM for the androgen receptor (e.g., AR-FL, AR-v7), or variant thereof, are selected in step (b) and/or (d). In certain embodiments, one or more compounds having an affinity of approximately 1 μM to approximately 500 μM for the androgen receptor (e.g., AR-FL, AR-v7), or variant thereof, are selected instep (b) and/or (d). In certain embodiments, one or more compounds having an affinity of approximately 1 μM to approximately 100 μM for the androgen receptor (e.g., AR-FL, AR-v7), or variant thereof, are selected in step (b) and/or (d). In certain embodiments, one or more compounds having an affinity of approximately 1 μM to approximately 50 μM for the androgen receptor (e.g., AR-FL, AR-v7). or variant thereof, are selected in steps (b) and/or (d). In certain embodiments, one or more compounds having an affinity of approximately 1 μM to approximately 10 μM for the androgen receptor (e.g., AR-FL, AR-v7), or variant thereof, are selected in step (b) and/or (d). In some aspects, step (d) of the method further comprises (e) measuring a first PSA expression level and a second PSA expression level with qPCR, wherein the first PSA expression level is the PSA expression level of cells exposed to a selected compound and the second PSA expression level is the PSA expression level of untreated cells; (f) comparing said first PSA expression level and said second PSA expression level; and (g) selecting one or more compounds that reduce the first PSA expression level by at least 10% compared to the second PSA expression level. In certain embodiments, one or more compounds that reduce the first PSA expression level by at least 20%, 30%, 40%, 50%, 60%, 70%, 80%, 90%, or 99% compared to the second PSA expression level are selected in step (g). In certain embodiments, one or more compounds that reduce the first PSA expression level by at least 40% compared to the second PSA expression level are selected in step (g).

In one aspect, the present disclosure provides methods of identifying an androgen receptor modulator, the methods comprising: (a) contacting a plurality of compounds with an androgen receptor, or variant thereof; (b) selecting one or more compounds that bind to the androgen receptor, or variant thereof; (c) contacting the one or more selected compounds that bind the androgen receptor, or variant thereof, individually with cells expressing an androgen receptor, or variant thereof; and (d) selecting one or more compounds that reduce prostate-specific antigen (PSA) expression in the cells. In certain embodiments, the plurality of compounds is provided in a small molecule microarray (SMM). The small molecule microarray may be comprised of approximately 1,000 to approximately 50,000 compounds to be screened against the androgen receptor, or variant thereof. In certain embodiments, the small molecule microarray may be comprised of approximately 50,000 compounds to be screened against the androgen receptor, or variant thereof. In certain embodiments, one or more compounds having an affinity of at least 1 mM for the androgen receptor (e.g., AR-FL, AR-v7), or variant thereof, are selected in step (b) and/or (d). In certain embodiments, one or more compounds having an affinity of approximately 5 nM to approximately 1 mM for the androgen receptor (e.g., AR-FL, AR-v7), or variant thereof, are selected in step (b) and/or (d). In certain embodiments, one or more compounds having an affinity of approximately 1 μM to approximately 1 mM for the androgen receptor (e.g., AR-FL, AR-v7), or variant thereof, are selected in step (b) and/or (d). In certain embodiments, one or more compounds having an affinity of approximately 1 μM to approximately 500 μM for the androgen receptor (e.g., AR-FL, AR-v7), or variant thereof, are selected instep (b) and/or (d). In certain embodiments, one or more compounds having an affinity of approximately 1 μM to approximately 100 μM for the androgen receptor (e.g., AR-FL, AR-v7), or variant thereof, are selected in step (b) and/or (d). In certain embodiments, one or more compounds having an affinity of approximately 1 μM to approximately 50 μM for the androgen receptor (e.g., AR-FL, AR-v7). or variant thereof, are selected in steps (b) and/or (d). In certain embodiments, one or more compounds having an affinity of approximately 1 μM to approximately 10 μM for the androgen receptor (e.g., AR-FL, AR-v7), or variant thereof, are selected in step (b) and/or (d). In some aspects, step (d) of the method further comprises (e) measuring a first PSA expression level and a second PSA expression level with qPCR, wherein the first PSA expression level is the PSA expression level of cells contacted with a selected compound and the second PSA expression level is the PSA expression level of untreated cells; (f) comparing said first PSA expression level and said second PSA expression level; and (g) selecting one or more compounds that reduce the first PSA expression level by at least 10% compared to the second PSA expression level. In certain embodiments, one or more compounds that reduce the first PSA expression level by at least 20%, 30%, 40%, 50%, 60%, 70%, 80%, 90%, or 99% compared to the second PSA expression level are selected in step (g). In certain embodiments, one or more compounds that reduce the first PSA expression level by at least 40% compared to the second PSA expression level are selected in step (g).

In another aspect, the present disclosure provides methods of identifying an androgen receptor modulator, the methods comprising: (a) exposing a plurality of compounds to an androgen receptor, or variant thereof; (b) selecting one or more compounds that bind to the androgen receptor, or variant thereof; (c) exposing the one or more selected compounds that bind the androgen receptor, or variant thereof, individually to cells expressing: i) an androgen receptor, or variant thereof, and ii) a reporter protein; and (d) selecting one or more compounds that reduce reporter protein activity level. In certain embodiments, the plurality of compounds is provided in a small molecule microarray (SMM). The small molecule microarray may be comprised of approximately 1,000 to approximately 50,000 compounds to be screened against the androgen receptor, or variant thereof. In certain embodiments, the small molecule microarray may be comprised of approximately 50,000 compounds to be screened against the androgen receptor, or variant thereof. In certain embodiments, one or more compounds having an affinity of at least 1 mM for the androgen receptor (e.g., AR-FL, AR-v7), or variant thereof, are selected in step (b) and/or (d). In certain embodiments, one or more compounds having an affinity of approximately 5 nM to approximately 1 mM for the androgen receptor (e.g., AR-FL, AR-v7), or variant thereof, are selected in step (b) and/or (d). In certain embodiments, one or more compounds having an affinity of approximately 1 μM to approximately 1 mM for the androgen receptor (e.g., AR-FL, AR-v7), or variant thereof, are selected in step (b) and/or (d). In certain embodiments, one or more compounds having an affinity of approximately 1 μM to approximately 500 μM for the androgen receptor (e.g., AR-FL, AR-v7), or variant thereof, are selected in step (b) and/or (d). In certain embodiments, one or more compounds having an affinity of approximately 1 μM to approximately 100 μM for the androgen receptor (e.g., AR-FL, AR-v7), or variant thereof, are selected in step (b) and/or (d). In certain embodiments, one or more compounds having an affinity of approximately 1 μM to approximately 50 μM for the androgen receptor (e.g., AR-FL, AR-v7), or variant thereof, are selected in step (b) and/or (d). In certain embodiments, one or more compounds having an affinity of approximately 1 μM to approximately 10 μM for the androgen receptor (e.g., AR-FL, AR-v7), or variant thereof, are selected in step (b) and/or (d). In some aspects, step (d) of the method further comprises (e) measuring a first reporter protein activity level and a second reporter protein activity level, wherein the first reporter protein activity level is the reporter protein activity level of cells treated with a compound selected for binding to an androgen receptor, or variant thereof, and the second reporter protein activity level is the reporter protein activity level of untreated cells, and wherein the first reporter protein activity level and the second reporter activity level are relative luciferase activity; (f) comparing said first reporter protein activity level and said second reporter protein activity level; and (g) selecting one or more compounds that reduce the first reporter protein activity level by at least 10% compared to the second reporter protein activity level. In an embodiment, one or more compounds that reduce the first reporter protein activity level by at least 20%, 30%, 40%, 50%, 60%, 70%, 80%, 90%, or 99% compared to the second reporter protein activity level are selected in step (g). In certain embodiments, one or more compounds that reduce the first reporter protein activity level by at least 40% compared to the second reporter protein activity level are selected in step (g). In an embodiment, the reporter protein is a luciferase (e.g., firefly luciferase, Renilla luciferase), beta-galactosidase, or a fluorescent protein (e.g., green fluorescent protein (GFP), red fluorescent protein (RFP), etc.). In an embodiment, the reporter protein is a luciferase. In certain embodiments, the luciferase is firefly luciferase. In certain embodiments, the luciferase is Renilla luciferase. In an embodiment, more than one type of reporter protein may be expressed in a cell (e.g., a reporter system). In an embodiment, the first reporter protein activity level and the second reporter protein activity level are measured using bioluminescence. In certain embodiments, the first reporter protein activity level and the second reporter protein activity level are measured using bioluminescence, wherein the bioluminescence source is luciferase. The relative luciferase activity is the luciferase activity of a first luciferase as compared to the activity of a second luciferase, wherein expression of the first luciferase is under control of the androgen receptor, or variant thereof, and expression of the second luciferase is under control of a constitutively active promoter. In certain embodiments, the first luciferase is firefly luciferase. In certain embodiments, the second luciferase is Renilla luciferase. In certain embodiments, the firefly luciferase is under control of an MMTV promoter that contains one or more androgen response element (ARE). In certain embodiments, the Renilla luciferase is under the control of a constitutively active CMV promoter.

In another aspect, the present disclosure provides methods of identifying an androgen receptor modulator, the methods comprising: (a) contacting a plurality of compounds with an androgen receptor, or variant thereof; (b) selecting one or more compounds that bind to the androgen receptor, or variant thereof; (c) contacting the one or more selected compounds that bind the androgen receptor, or variant thereof, individually with cells expressing: i) an androgen receptor, or variant thereof, and ii) a reporter protein; and (d) selecting one or more compounds that reduce reporter protein activity level. In certain embodiments, the plurality of compounds is provided in a small molecule microarray (SMM). The small molecule microarray may be comprised of approximately 1,000 to approximately 50,000 compounds to be screened against the androgen receptor, or variant thereof. In certain embodiments, the small molecule microarray may be comprised of approximately 50,000 compounds to be screened against the androgen receptor, or variant thereof. In certain embodiments, one or more compounds having an affinity of at least 1 mM for the androgen receptor (e.g., AR-FL, AR-v7), or variant thereof, are selected in step (b) and/or (d). In certain embodiments, one or more compounds having an affinity of approximately 5 nM to approximately 1 mM for the androgen receptor (e.g., AR-FL, AR-v7), or variant thereof, are selected in step (b) and/or (d). In certain embodiments, one or more compounds having an affinity of approximately 1 μM to approximately 1 mM for the androgen receptor (e.g., AR-FL, AR-v7), or variant thereof, are selected in step (b) and/or (d). In certain embodiments, one or more compounds having an affinity of approximately 1 μM to approximately 500 μM for the androgen receptor (e.g., AR-FL, AR-v7), or variant thereof, are selected in step (b) and/or (d). In certain embodiments, one or more compounds having an affinity of approximately 1 μM to approximately 100 μM for the androgen receptor (e.g., AR-FL, AR-v7), or variant thereof, are selected in step (b) and/or (d). In certain embodiments, one or more compounds having an affinity of approximately 1 μM to approximately 50 μM for the androgen receptor (e.g., AR-FL, AR-v7), or variant thereof, are selected in step (b) and/or (d). In certain embodiments, one or more compounds having an affinity of approximately 1 μM to approximately 10 μM for the androgen receptor (e.g., AR-FL, AR-v7), or variant thereof, are selected in step (b) and/or (d). In some aspects, step (d) of the method further comprises (e) measuring a first reporter protein activity level and a second reporter protein activity level, wherein the first reporter protein activity level is the reporter protein activity level of cells treated with a compound selected for binding to an androgen receptor, or variant thereof, and the second reporter protein activity level is the reporter protein activity level of untreated cells, and wherein the first reporter protein activity level and the second reporter activity level are relative luciferase activity; (f) comparing said first reporter protein activity level and said second reporter protein activity level; and (g) selecting one or more compounds that reduce the first reporter protein activity level by at least 10% compared to the second reporter protein activity level. In an embodiment, one or more compounds that reduce the first reporter protein activity level by at least 20%, 30%, 40%, 50%, 60%, 70%, 80%, 90%, or 99% compared to the second reporter protein activity level are selected in step (g). In certain embodiments, one or more compounds that reduce the first reporter protein activity level by at least 40% compared to the second reporter protein activity level are selected in step (g). In an embodiment, the reporter protein is a luciferase (e.g., firefly luciferase, Renilla luciferase), beta-galactosidase, or a fluorescent protein (e.g., green fluorescent protein (GFP), red fluorescent protein (RFP), etc.). In an embodiment, the reporter protein is a luciferase. In certain embodiments, the luciferase is firefly luciferase. In certain embodiments, the luciferase is Renilla luciferase. In an embodiment, more than one type of reporter protein may be expressed in a cell (e.g., a reporter system). In an embodiment, the first reporter protein activity level and the second reporter protein activity level are measured using bioluminescence. In certain embodiments, the first reporter protein activity level and the second reporter protein activity level are measured using bioluminescence, wherein the bioluminescence source is luciferase. The relative luciferase activity is the luciferase activity of a first luciferase as compared to the activity of a second luciferase, wherein expression of the first luciferase is under control of the androgen receptor, or variant thereof, and expression of the second luciferase is under control of a constitutively active promoter. In certain embodiments, the first luciferase is firefly luciferase. In certain embodiments, the second luciferase is Renilla luciferase. In certain embodiments, the firefly luciferase is under control of an MMTV promoter that contains one or more androgen response element (ARE). In certain embodiments, the Renilla luciferase is under the control of a constitutively active CMV promoter.

In yet another aspect, the present disclosure provides methods of identifying an androgen receptor modulator, the methods comprising: (a) exposing a plurality of compounds to an androgen receptor, or variant thereof; (b) selecting one or more compounds that bind to the androgen receptor, or variant thereof; (c) exposing the one or more selected compounds that bind the androgen receptor, or variant thereof, individually to cells expressing an androgen receptor, or variant thereof; (d) selecting one or more compounds that reduce prostate-specific antigen (PSA) expression in the cells; (e) exposing the one or more compounds selected to reduce PSA expression individually to cells expressing: i) an androgen receptor or fragment thereof, and ii) a reporter protein; and (f) selecting one or more compounds that reduce reporter protein activity level. In an embodiment, the androgen receptor (e.g., AR-FL, AR-v7) is provided in the form of the purified androgen receptor, or variant thereof. In an embodiment, the androgen receptor (e.g., AR-FL, AR-v7) is provided in the form of a cell lysate comprising the androgen receptor, or variant thereof. In certain embodiments, the plurality of compounds is provided in a small molecule microarray (SMM). The small molecule microarray may be comprised of approximately 1,000 to approximately 50,000 compounds to be screened against the androgen receptor, or variant thereof. In certain embodiments, the small molecule microarray may be comprised of approximately 50,000 compounds to be screened against the androgen receptor, or variant thereof. In certain embodiments, one or more compounds having an affinity of at least 1 mM for the androgen receptor (e.g., AR-FL, AR-v7), or variant thereof, are selected in step (b), (d), and/or (f). In certain embodiments, one or more compounds having an affinity of approximately 5 nM to approximately 1 mM for the androgen receptor (e.g., AR-FL, AR-v7), or variant thereof, are selected in steps (b), (d), and/or (f). In certain embodiments, one or more compounds having an affinity of approximately 1 μM to approximately 1 mM for the androgen receptor (e.g., AR-FL, AR-v7), or variant thereof, are selected in step (b), (d), and/or (f). In certain embodiments, one or more compounds having an affinity of approximately 1 μM to approximately 500 μM for the androgen receptor (e.g., AR-FL, AR-v7), or variant thereof, are selected in step (b), (d), and/or (f). In certain embodiments, one or more compounds having an affinity of approximately 1 μM to approximately 100 μM for the androgen receptor (e.g., AR-FL, AR-v7), or variant thereof are selected in step (b), (d), and/or (f). In certain embodiments, one or more compounds having an affinity of approximately 1 μM to approximately 50 μM for the androgen receptor (e.g., AR-FL, AR-v7), or variant thereof, are selected in step (b), (d), and/or (f). In certain embodiments, one or more compounds having an affinity of approximately 1 μM to approximately 10 μM for the androgen receptor (e.g., AR-FL, AR-v7), or variant thereof, are selected in step (b), (d), and/or (f). In yet another aspect, step (d) of the method further comprises (g) measuring a first PSA expression level and a second PSA expression level with qPCR, wherein the first PSA expression level is the PSA expression level of cells exposed to a selected compound and the second PSA expression level is the PSA expression level of untreated cells; (h) comparing said first PSA expression level and said second PSA expression level; and (i) selecting one or more compounds that reduce the first PSA expression level by at least 10% compared to the second PSA expression level. In an embodiment, one or more compounds that reduce the first PSA expression level by at least 20%, 30%, 40%, 50%, 60%, 70%, 80%, 90%, or 99% compared to the second PSA expression level are selected in step (i). In certain embodiments, one or more compounds that reduce the first PSA expression level by at least 40% compared to the second PSA expression level are selected in step (i). In yet another aspect, step (f) of the method further comprises (j) measuring a first reporter protein activity level and a second reporter protein activity level, wherein the first reporter protein activity level is the reporter protein activity level of cells treated with a compound selected for binding to an androgen receptor, or variant thereof, and the second reporter protein activity level is the reporter protein activity level of untreated cells, and wherein the first reporter protein activity level and the second reporter activity level are relative luciferase activity; (k) comparing said first reporter protein activity level and said second reporter protein activity level; and (l) selecting one or more compounds that reduce the first reporter protein activity level by at least 10% compared to the second reporter protein activity level. In an embodiment, one or more compounds that reduce the first reporter protein activity level by at least 20%, 30%, 40%, 50%, 60%, 70%, 80%, 90%, or 99% compared to the second reporter protein activity level are selected in step (l). In certain embodiments, one or more compounds that reduce the first reporter protein activity level by at least 40% compared to the second reporter protein activity level are selected in step (l). In an embodiment, the reporter protein is a luciferase (e.g., firefly luciferase, Renilla luciferase), beta-galactosidase, or a fluorescent protein (e.g., green fluorescent protein (GFP), red fluorescent protein (RFP), etc.). In an embodiment, the reporter protein is a luciferase. In certain embodiments, the luciferase is firefly luciferase. In certain embodiments, the luciferase is Renilla luciferase. In an embodiment, more than one type of reporter protein may be expressed in a cell (e.g., a reporter system). In an embodiment, the first reporter protein activity level and the second reporter protein activity level are measured using bioluminescence. In certain embodiments, the first reporter protein activity level and the second reporter protein activity level are measured using a bioluminescence signal from luciferase. The relative luciferase activity is the luciferase activity of a first luciferase as compared to the activity of a second luciferase, wherein expression of the first luciferase is under control of the androgen receptor, or variant thereof, and expression of the second luciferase is under control of a constitutively active promoter. In certain embodiments, the first luciferase is firefly luciferase. In certain embodiments, the second luciferase is Renilla luciferase. In certain embodiments, the firefly luciferase is under control of an MMTV promoter that contains one or more androgen response element (ARE). In certain embodiments, the Renilla luciferase is under the control of a constitutively active CMV promoter.

In yet another aspect, the present disclosure provides methods of identifying an androgen receptor modulator, the methods comprising: (a) contacting a plurality of compounds with an androgen receptor, or variant thereof; (b) selecting one or more compounds that bind to the androgen receptor, or variant thereof; (c) contacting the one or more selected compounds that bind the androgen receptor, or variant thereof, individually with cells expressing an androgen receptor, or variant thereof; (d) selecting one or more compounds that reduce prostate-specific antigen (PSA) expression in the cells; (e) contacting the one or more compounds selected to reduce PSA expression individually with cells expressing: i) an androgen receptor or fragment thereof, and ii) a reporter protein; and (f) selecting one or more compounds that reduce reporter protein activity level. In an embodiment, the androgen receptor (e.g., AR-FL, AR-v7) is provided in the form of the purified androgen receptor, or variant thereof. In an embodiment, the androgen receptor (e.g., AR-FL, AR-v7) is provided in the form of a cell lysate comprising the androgen receptor, or variant thereof. In certain embodiments, the plurality of compounds is provided in a small molecule microarray (SMM). The small molecule microarray may be comprised of approximately 1,000 to approximately 50,000 compounds to be screened against the androgen receptor, or variant thereof. In certain embodiments, the small molecule microarray may be comprised of approximately 50,000 compounds to be screened against the androgen receptor, or variant thereof. In certain embodiments, one or more compounds having an affinity of at least 1 mM for the androgen receptor (e.g., AR-FL, AR-v7), or variant thereof, are selected in step (b), (d), and/or (f). In certain embodiments, one or more compounds having an affinity of approximately 5 nM to approximately 1 mM for the androgen receptor (e.g., AR-FL, AR-v7), or variant thereof, are selected in steps (b), (d), and/or (f). In certain embodiments, one or more compounds having an affinity of approximately 1 μM to approximately 1 mM for the androgen receptor (e.g., AR-FL, AR-v7), or variant thereof, are selected in step (b), (d), and/or (f). In certain embodiments, one or more compounds having an affinity of approximately 1 μM to approximately 500 μM for the androgen receptor (e.g., AR-FL, AR-v7), or variant thereof, are selected in step (b), (d), and/or (f). In certain embodiments, one or more compounds having an affinity of approximately 1 μM to approximately 100 μM for the androgen receptor (e.g., AR-FL, AR-v7), or variant thereof are selected in step (b), (d), and/or (f). In certain embodiments, one or more compounds having an affinity of approximately 1 μM to approximately 50 μM for the androgen receptor (e.g., AR-FL, AR-v7), or variant thereof, are selected in step (b), (d), and/or (f). In certain embodiments, one or more compounds having an affinity of approximately 1 μM to approximately 10 μM for the androgen receptor (e.g., AR-FL, AR-v7), or variant thereof, are selected in step (b), (d), and/or (f). In yet another aspect, step (d) of the method further comprises (g) measuring a first PSA expression level and a second PSA expression level with qPCR, wherein the first PSA expression level is the PSA expression level of cells contacted with a selected compound and the second PSA expression level is the PSA expression level of untreated cells; (h) comparing said first PSA expression level and said second PSA expression level; and (i) selecting one or more compounds that reduce the first PSA expression level by at least 10% compared to the second PSA expression level. In an embodiment, one or more compounds that reduce the first PSA expression level by at least 20%, 30%, 40%, 50%, 60%, 70%, 80%, 90%, or 99% compared to the second PSA expression level are selected in step (i). In certain embodiments, one or more compounds that reduce the first PSA expression level by at least 40% compared to the second PSA expression level are selected in step (i). In yet another aspect, step (f) of the method further comprises (j) measuring a first reporter protein activity level and a second reporter protein activity level, wherein the first reporter protein activity level is the reporter protein activity level of cells treated with a compound selected for binding to an androgen receptor, or variant thereof, and the second reporter protein activity level is the reporter protein activity level of untreated cells, and wherein the first reporter protein activity level and the second reporter activity level are relative luciferase activity; (k) comparing said first reporter protein activity level and said second reporter protein activity level; and (l) selecting one or more compounds that reduce the first reporter protein activity level by at least 10% compared to the second reporter protein activity level. In an embodiment, one or more compounds that reduce the first reporter protein activity level by at least 20%, 30%, 40%, 50%, 60%, 70%, 80%, 90%, or 99% compared to the second reporter protein activity level are selected in step (l). In certain embodiments, one or more compounds that reduce the first reporter protein activity level by at least 40% compared to the second reporter protein activity level are selected in step (l). In an embodiment, the reporter protein is a luciferase (e.g., firefly luciferase, Renilla luciferase), beta-galactosidase, or a fluorescent protein (e.g., green fluorescent protein (GFP), red fluorescent protein (RFP), etc.). In an embodiment, the reporter protein is a luciferase. In certain embodiments, the luciferase is firefly luciferase. In certain embodiments, the luciferase is Renilla luciferase. In an embodiment, more than one type of reporter protein may be expressed in a cell (e.g., a reporter system). In an embodiment, the first reporter protein activity level and the second reporter protein activity level are measured using bioluminescence. In certain embodiments, the first reporter protein activity level and the second reporter protein activity level are measured using a bioluminescence signal from luciferase. The relative luciferase activity is the luciferase activity of a first luciferase as compared to the activity of a second luciferase, wherein expression of the first luciferase is under control of the androgen receptor, or variant thereof, and expression of the second luciferase is under control of a constitutively active promoter. In certain embodiments, the first luciferase is firefly luciferase. In certain embodiments, the second luciferase is Renilla luciferase. In certain embodiments, the firefly luciferase is under control of an MMTV promoter that contains one or more androgen response element (ARE). In certain embodiments, the Renilla luciferase is under the control of a constitutively active CMV promoter.

In another aspect, the present disclosure provides methods for confirming the activity of one or more compounds having an affinity for an androgen receptor, or variant thereof, the methods comprising (a) exposing the one or more selected compounds that bind the androgen receptor, or variant thereof, individually to cells expressing an androgen receptor, or variant thereof; (b) measuring a first PSA expression level and a second PSA expression level with qPCR, wherein the first PSA expression level is the PSA expression level of cells exposed to a selected compound and the second PSA expression level is the PSA expression level of untreated cells; (c) comparing said first PSA expression level and said second PSA expression level; and (d) determining that the first PSA expression level is lower as compared to the second PSA expression level, thereby confirming the activity of one or more compounds. One or more compounds that reduce the first PSA expression level by at least 10%, 20%, 30%, 40%, 50%, 60%, 70%, 80%, 90%, or 99% compared to the second PSA expression level are considered active compounds that modulate the activity of the androgen receptor, or variant thereof.

In another aspect, the present disclosure provides methods for confirming the activity of one or more compounds having an affinity for an androgen receptor, or variant thereof, the methods comprising (a) contacting the one or more selected compounds that bind the androgen receptor, or variant thereof, individually with cells expressing an androgen receptor, or variant thereof; (b) measuring a first PSA expression level and a second PSA expression level with qPCR, wherein the first PSA expression level is the PSA expression level of cells contacted with a selected compound and the second PSA expression level is the PSA expression level of untreated cells; (c) comparing said first PSA expression level and said second PSA expression level; and (d) determining that the first PSA expression level is lower as compared to the second PSA expression level, thereby confirming the activity of one or more compounds. One or more compounds that reduce the first PSA expression level by at least 10%, 20%, 30%, 40%, 50%, 60%, 70%, 80%, 90%, or 99% compared to the second PSA expression level are considered active compounds that modulate the activity of the androgen receptor, or variant thereof.

In another aspect, the present disclosure provides methods for confirming the activity of one or more compounds with an affinity for an androgen receptor, or variant thereof, the method comprising (a) exposing the one or more selected compounds that bind the androgen receptor, or variant thereof, individually to cells expressing: i) an androgen receptor, or variant thereof, and ii) a reporter protein; (b) measuring a first reporter protein activity level and a second reporter protein activity level, wherein the first reporter protein activity level is the reporter protein activity level of cells treated with a compound selected for binding to an androgen receptor, or variant thereof, and the second reporter protein activity level is the reporter protein activity level of untreated cells, and wherein the first reporter protein activity level and the second reporter activity level are relative luciferase activity; (c) comparing said first reporter protein activity level and said second reporter protein activity level; and (d) determining that the first reporter protein activity level is lower as compared to the second reporter protein activity level, thereby confirming the activity of one or more compounds. One or more compounds that reduce the first reporter protein activity level by at least 10%, 20%, 30%, 40%, 50%, 60%, 70%, 80%, 90%, or 99% compared to the second reporter activity level are considered active compounds that modulate the activity of the androgen receptor, or variant thereof.

In another aspect, the present disclosure provides methods for confirming the activity of one or more compounds with an affinity for an androgen receptor, or variant thereof, the method comprising (a) contacting the one or more selected compounds that bind the androgen receptor, or variant thereof, individually with cells expressing: i) an androgen receptor, or variant thereof, and ii) a reporter protein; (b) measuring a first reporter protein activity level and a second reporter protein activity level, wherein the first reporter protein activity level is the reporter protein activity level of cells treated with a compound selected for binding to an androgen receptor, or variant thereof, and the second reporter protein activity level is the reporter protein activity level of untreated cells, and wherein the first reporter protein activity level and the second reporter activity level are relative luciferase activity; (c) comparing said first reporter protein activity level and said second reporter protein activity level; and (d) determining that the first reporter protein activity level is lower as compared to the second reporter protein activity level, thereby confirming the activity of one or more compounds. One or more compounds that reduce the first reporter protein activity level by at least 10%, 20%, 30%, 40%, 50%, 60%, 70%, 80%, 90%, or 99% compared to the second reporter activity level are considered active compounds that modulate the activity of the androgen receptor, or variant thereof.

Throughout the screening methods described herein, a cell viability assay may be used to determine the number of viable cells in a culture, for example after treatment with a potentially cytotoxic agent or high dosage. A cell viability assay generally measures the amount of ATP present, an indicator of metabolically active cells, in a culture. One such assay is a CellTiter-Glo® assay (Promega Corp.), and a variety of other cell viability assays are known in the art.

### Androgen Receptor, Androgen Receptor Genes and Gene Products, and Androgen Receptor Splice Variants

As discussed above, the screening methods disclosed herein take advantage of screening an androgen receptor, or variant thereof, and have been used to identify compounds that modulate the acvitity of the androgen receptor, or variant thereof. One mechanism for resistance to traditional prostate cancer and CRPC therapies arises from the expression of androgen receptor variants (AR-vs), which provide a mechanism for continual androgen receptor-mediated gene expression. In some cases, the AR-v is an androgen receptor that lacks a LBD, rendering it unresponsive to traditional therapies, such as enzalutamide. These AR-vs are commonly constitutively active and effectively mimic full length androgen receptor (AR-FL) in their ability to transactivate androgen response elements (AREs) without androgen stimulation. The screening methods disclosed herein have identified compounds that target AR-vs (e.g., AR-v7) that are capable of promoting continual androgen-receptor mediated gene expression, providing compounds that may be used for treating subjects with metastatic or castration-resistant prostate cancer for which there are no other effective therapies.

Sequences of the androgen receptor gene products of interest herein often comprise or consist of sequences encoded by human androgen receptor genes, although sequences of non-human mammalian homologs may be used in certain embodiments. In general, the sequence of an androgen receptor protein or androgen receptor RNA often comprises or consists of a sequence of a human androgen receptor. In certain embodiments, the sequence of a gene product of an androgen receptor gene comprises or consists of a naturally occurring sequence. It will be appreciated that a genetic locus may have more than one sequence or allele in a population of individuals. In some embodiments a naturally occurring sequence is a standard sequence. Unless otherwise indicated, a sequence listed in the Reference Sequence (RefSeq) Database as a reference sequence for a protein that is referred to herein by a particular name, abbreviation, or symbol, is considered to be a “standard sequence.” If a sequence has been updated subsequent to the time of the present disclosure a version current at the time of the present disclosure or an updated version thereof may be used in certain embodiments. It will be appreciated that a genetic locus may have more than one sequence or allele in a population of individuals. In some embodiments a naturally occurring sequence differs from a standard sequence at one or more amino acid positions. A naturally occurring polynucleotide or polypeptide whose sequence differs from a standard sequence and that performs the normal function(s) of the polynucleotide or polypeptide may be referred to as having a “normal sequence”.

In certain embodiments, the androgen receptor, or variant thereof, is the full-length androgen receptor (AR-FL). The AR-FL may be a mammalian (e.g., human) AR-FL. In certain embodiments the sequence of an androgen receptor or fragment thereof used in the compositions and methods described herein comprises the sequence of a naturally occurring androgen receptor protein or a biologically active variant thereof. A biologically active variant of an androgen receptor protein may contain one or more additions, substitutions, and/or deletions relative to the sequence of a naturally occurring androgen receptor protein. In some embodiments the sequence of an androgen receptor protein comprises a standard androgen receptor sequence. AR-FL is coded from eight exons and comprises four functional domains: an N-terminal domain (NTD), a DNA-binding domain (DBD), a hinge region, and a C-terminal ligand binding domain (LBD). On the AR gene locus, exon 1 encodes the NTD, exons 2 and 3 encode the DBD, exon 4 encodes the hinge region, and exons 5-8 encode the LBD. AR-FL is normally 920 amino acids in length and has the following standard amino acid sequence (GenBank and NCBI Reference Sequence Accession Number: NP_000035.2):

(SEQ ID NO: 1)
MEVQLGLGRVYPRPPSKTYRGAFQNLFQSVREVIQNPGPRHPEAASAAPP
GASLLLLQQQQQQQQQQQQQQQQQQQQQQQETSPRQQQQQQGEDGSPQAH
RRGPTGYLVLDEEQQPSQPQSALECHPERGCVPEPGAAVAASKGLPQQLP
APPDEDDSAAPSTLSLLGPTFPGLSSCSADLKDILSEASTMQLLQQQQQE
AVSEGSSSGRAREASGAPTSSKDNYLGGTSTISDNAKELCKAVSVSMGLG
VEALEHLSPGEQLRGDCMYAPLLGVPPAVRPTPCAPLAECKGSLLDDSAG
KSTEDTAEYSPFKGGYTKGLEGESLGCSGSAAAGSSGTLELPSTLSLYKS
GALDEAAAYQSRDYYNFPLALAGPPPPPPPPHPHARIKLENPLDYGSAWA
AAAAQCRYGDLASLHGAGAAGPGSGSPSAAASSWHTLFTAEEGQLYGPCG
GGGGGGGGGGGGGGGGGGGGGGEAGAVAPYGYTRPPQGLAGQESDFTAPD
VWYPGGMVSRVPYPSPTCVKSEMGPWMDSYSGPYGDMRLETARDHVLPID
YYFPPQKTCLICGDEASGCHYGALTCGSCKVFFKRAAEGKQKYLCASRND
CTIDKFRRKNCPSCRLRKCYEAGMTLGARKLKKLGNLKLQEEGEASSTTS
PTEETTQKLTVSHIEGYECQPIFLNVLEAIEPGVVCAGHDNNQPDSFAAL
LSSLNELGERQLVHVVKWAKALPGFRNLHVDDQMAVIQYSWMGLMVFAMG
WRSFTNVNSRMLYFAPDLVFNEYRMHKSRMYSQCVRMRHLSQEFGWLQIT
PQEFLCMKALLLFSIIPVDGLKNQKFFDELRMNYIKELDRIIACKRKNPT
SCSRRFYQLTKLLDSVQPIARELHQFTFDLLIKSHMVSVDFPEMMAEIIS
VQVPKILSGKVKPIYFHTQ

In certain embodiments, the present disclosure provides compositions and methods useful for modulating an androgen receptor, or variant thereof, wherein the androgen receptor, or variant thereof, is an androgen receptor variant (AR-v). The AR-v may be a mammalian (e.g., human) AR-v. In certain embodiments, compositions and methods are useful for modulating an androgen receptor or fragment thereof, wherein the androgen receptor or fragment thereof is an androgen receptor variant (AR-v) and wherein the AR-v lacks the ligand binding domain (LBD). In certain embodiments, the compositions and methods are useful for modulating an androgen receptor or fragment thereof, wherein the androgen receptor or fragment thereof is an androgen receptor variant (AR-v) and wherein the AR-v lacks the ligand binding domain (LBD) and the hinge domain. In certain embodiments, the compositions and methods are useful for modulating an androgen receptor or fragment thereof, wherein the androgen receptor or fragment thereof is an androgen receptor variant (AR-v) and wherein the AR-v comprises a N-terminal binding domain (NTD). In certain embodiments, the compositions and methods are useful for modulating an androgen receptor or fragment thereof, wherein the androgen receptor or fragment thereof is an androgen receptor variant (AR-v) and wherein the AR-v comprises a N-terminal binding domain (NTD) and a DNA-binding domain (DBD). In certain embodiments, the compositions and methods are useful for modulating an androgen receptor or fragment thereof, wherein the androgen receptor or fragment thereof is an androgen receptor variant (AR-v) and wherein the AR-v comprises a N-terminal binding domain (NTD) and a DNA-binding domain (DBD).

In some embodiments an androgen receptor protein is a mutant androgen receptor protein, e.g., the sequence of the protein comprises the sequence of a naturally occurring mutant form of androgen receptor. In some embodiments an androgen receptor harbors a mutation or genetic variation that is associated with an increased risk of developing metastatic or castration-resistant prostate cancer. A mutation may be in a coding sequence or regulatory region. Certain point mutations in androgen receptor (e.g., H874Y, T877A, T877S) are gain of function mutations frequently found in advanced prostate cancers and may play a role in tumor progression. In certain embodiments, an androgen receptor comprises a H874Y mutation. In certain embodiments, an androgen receptor comprises a T877A mutation. In certain embodiments, an androgen receptor comprises a T877S mutation. In certain embodiments a human subject harbors a H874Y, T877A, or T877S mutation in at least one allele of the gene encoding the androgen receptor. In certain embodiments a human prostate gland or prostate cancer cell harbors a H874Y, T877A, or T877S mutation in at least one allele of the gene encoding the androgen receptor

In some embodiments, an androgen receptor protein is a androgen receptor variant or fragment thereof. The term “variant” is used interchangeably herein with the terms “splice variant” and “split variant.” Splice variants arise through the process of alternative splicing, or differential splicing. When a mature, processed messenger RNA (mRNA) molecule comprises all eight of the exons on the AR gene locus, the full-length androgen receptor (AR-FL) protein is produced. When the AR gene locus is alternatively spliced, the resulting mRNA construct comprises a differential set of exons when compared to the full-length androgen receptor. Additionally, one or more cryptic exons (CEs) may be inserted into the mRNA of AR-vs. See, e.g., Lu, J., Van der Steen, T., and Tindall, D. J. (2015) Are androgen receptor variants a substitute for the full-length receptor? Nat Rev Urol, 12: 137-144, which is incorporated herein by reference in its entirety. Alternative splicing gives rise to androgen receptor variants (AR-vs). The 21 known AR-vs are AR23, ARQ640X, AR-v1 (AR4), AR-v2, AR-v3 (AR1/2/2b/AR6), AR-v4 (AR1/2/3/2b, AR5), AR-v5, AR-v6, AR-v7 (AR3), AR-v8, AR-v9, AR-v10, AR-v11, AR-v12 (ARV567es), AR-v13, AR-v14, AR-v15, AR-v16, AR-v18, AR8, and AR45. The compounds presented herein may be useful in modulating the activity of any AR-v comprising exons 1, 2, and 3. Amino acid sequences of the AR-vs are given below.

AR23
(SEQ ID NO: 4)
MEVQLGLGRVYPRPPSKTYRGAFQNLFQSVREVIQNPGPRHPEAASAAPPGASLLLLQQ
QQQQQQQQQQQQQQQQQQQQQETSPRQQQQQQGEDGSPQAHRRGPTGYLVLDEEQQ
PSQPQSALECHPERGCVPEPGAAVAASKGLPQQLPAPPDEDDSAAPSTLSLLGPTFPGLSS
CSADLKDILSEASTMQLLQQQQQEAVSEGSSSGRAREASGAPTSSKDNYLGGTSTISDNA
KELCKAVSVSMGLGVEALEHLSPGEQLRGDCMYAPLLGVPPAVRPTPCAPLAECKGSL
LDDSAGKSTEDTAEYSPFKGGYTKGLEGESLGCSGSAAAGSSGTLELPSTLSLYKSGALD
EAAAYQSRDYYNFPLALAGPPPPPPPPHPHARIKLENPLDYGSAWAAAAAQCRYGDLAS
LHGAGAAGPGSGSPSAAASSSWHTLFTAEEGQLYGPCGGGGGGGGGGGGGGGGGGGG
GGGEAGAVAPYGYTRPPQGLAGQESDFTAPDVWYPGGMVSRVPYPSPTCVKSEMGPW
MDSYSGPYGDMRLETARDHVLPIDYYFPPQKTCLICGDEASGCHYGALTCGSCKVFFKR
AAEEIPEERDSGNSLSGLSTLVFVLPGKQKYLCASRNDCTIDKFRRKNCPSCRLRKCYEA
GMTLGARKLKKLGNLKLQEEGEASSTTSPTEETTQKLTVSHIEGYECQPIFLNVLEAIEPG
VVCAGHDNNQPDSFAALLSSLNELGERQLVHVVKWAKALPGFRNLHVDDQMAVIQYS
WMGLMVFAMGWRSFTNVNSRMLYFAPDLVFNEYRMHKSRMYSQCVRMRHLSQEFG
WLQITPQEFLCMKALLLFSIIPVDGLKNQKFFDELRMNYIKELDRIIACKRKNPTSCSRRF
YQLTKLLDSVQPIARELHQFTFDLLIKSHMVSVDFPEMMAEIISVQVPKILSGKVKPIYFH
TQ
ARQ640X
(SEQ ID NO: 5)
MEVQLGLGRVYPRPPSKTYRGAFQNLFQSVREVIQNPGPRHPEAASAAPPGASLLLLQQ
QQQQQQQQQQQQQQQQQQQQQETSPRQQQQQQGEDGSPQAHRRGPTGYLVLDEEQQ
PSQPQSALECHPERGCVPEPGAAVAASKGLPQQLPAPPDEDDSAAPSTLSLLGPTFPGLSS
CSADLKDILSEASTMQLLQQQQQEAVSEGSSSGRAREASGAPTSSKDNYLGGTSTISDNA
KELCKAVSVSMGLGVEALEHLSPGEQLRGDCMYAPLLGVPPAVRPTPCAPLAECKGSL
LDDSAGKSTEDTAEYSPFKGGYTKGLEGESLGCSGSAAAGSSGTLELPSTLSLYKSGALD
EAAAYQSRDYYNFPLALAGPPPPPPPPHPHARIKLENPLDYGSAWAAAAAQCRYGDLAS
LHGAGAAGPGSGSPSAAASSSWHTLFTAEEGQLYGPCGGGGGGGGGGGGGGGGGGGG
GGGEAGAVAPYGYTRPPQGLAGQESDFTAPDVWYPGGMVSRVPYPSPTCVKSEMGPW
MDSYSGPYGDMRLETARDHVLPIDYYFPPQKTCLICGDEASGCHYGALTCGSCKVFFKR
AAEGKQKYLCASRNDCTIDKFRRKNCPSCRLRKCYEAGMTLGARKLKKLGNLKL
AR-v1 (AR4) (GenBank and NCBI Reference Sequence Accession
Number: NP_001334992.1)
(SEQ ID NO: 6)
MEVQLGLGRVYPRPPSKTYRGAFQNLFQSVREVIQNPGPRHPEAASAAPPGASLLLLQQ
QQQQQQQQQQQQQQQQQQQQQETSPRQQQQQQGEDGSPQAHRRGPTGYLVLDEEQQ
PSQPQSALECHPERGCVPEPGAAVAASKGLPQQLPAPPDEDDSAAPSTLSLLGPTFPGLSS
CSADLKDILSEASTMQLLQQQQQEAVSEGSSSGRAREASGAPTSSKDNYLGGTSTISDNA
KELCKAVSVSMGLGVEALEHLSPGEQLRGDCMYAPLLGVPPAVRPTPCAPLAECKGSL
LDDSAGKSTEDTAEYSPFKGGYTKGLEGESLGCSGSAAAGSSGTLELPSTLSLYKSGALD
EAAAYQSRDYYNFPLALAGPPPPPPPPHPHARIKLENPLDYGSAWAAAAAQCRYGDLAS
LHGAGAAGPGSGSPSAAASSSWHTLFTAEEGQLYGPCGGGGGGGGGGGGGGGGGGGG
GGGEAGAVAPYGYTRPPQGLAGQESDFTAPDVWYPGGMVSRVPYPSPTCVKSEMGPW
MDSYSGPYGDMRLETARDHVLPIDYYFPPQKTCLICGDEASGCHYGALTCGSCKVFFKR
AAEGKQKYLCASRNDCTIDKFRRKNCPSCRLRKCYEAGMTLGAAVVVSERILRVFGVS
EWLP
AR-v2
(SEQ ID NO: 7)
MEVQLGLGRVYPRPPSKTYRGAFQNLFQSVREVIQNPGPRHPEAASAAPPGASLLLLQQ
QQQQQQQQQQQQQQQQQQQQQETSPRQQQQQQGEDGSPQAHRRGPTGYLVLDEEQQ
PSQPQSALECHPERGCVPEPGAAVAASKGLPQQLPAPPDEDDSAAPSTLSLLGPTFPGLSS
CSADLKDILSEASTMQLLQQQQQEAVSEGSSSGRAREASGAPTSSKDNYLGGTSTISDNA
KELCKAVSVSMGLGVEALEHLSPGEQLRGDCMYAPLLGVPPAVRPTPCAPLAECKGSL
LDDSAGKSTEDTAEYSPFKGGYTKGLEGESLGCSGSAAAGSSGTLELPSTLSLYKSGALD
EAAAYQSRDYYNFPLALAGPPPPPPPPHPHARIKLENPLDYGSAWAAAAAQCRYGDLAS
LHGAGAAGPGSGSPSAAASSSWHTLFTAEEGQLYGPCGGGGGGGGGGGGGGGGGGGG
GGGEAGAVAPYGYTRPPQGLAGQESDFTAPDVWYPGGMVSRVPYPSPTCVKSEMGPW
MDSYSGPYGDMRLETARDHVLPIDYYFPPQKTCLICGDEASGCHYGALTCGSCKVFFKR
AAEGKQKYLCASRNDCTIDKFRRKNCPSCRLRKCYEAGMTLGGKQKYLCASRNDCTID
KFRRKNCPSCRLRKCYEAGMTLGAAVVVSERILRVFGVSEWLP
AR-v3 (AR1/2/2b/AR6)
(SEQ ID NO: 8)
MEVQLGLGRVYPRPPSKTYRGAFQNLFQSVREVIQNPGPRHPEAASAAPPGASLLLLQQ
QQQQQQQQQQQQQQQQQQQQQETSPRQQQQQQGEDGSPQAHRRGPTGYLVLDEEQQ
PSQPQSALECHPERGCVPEPGAAVAASKGLPQQLPAPPDEDDSAAPSTLSLLGPTFPGLSS
CSADLKDILSEASTMQLLQQQQQEAVSEGSSSGRAREASGAPTSSKDNYLGGTSTISDNA
KELCKAVSVSMGLGVEALEHLSPGEQLRGDCMYAPLLGVPPAVRPTPCAPLAECKGSL
LDDSAGKSTEDTAEYSPFKGGYTKGLEGESLGCSGSAAAGSSGTLELPSTLSLYKSGALD
EAAAYQSRDYYNFPLALAGPPPPPPPPHPHARIKLENPLDYGSAWAAAAAQCRYGDLAS
LHGAGAAGPGSGSPSAAASSSWHTLFTAEEGQLYGPCGGGGGGGGGGGGGGGGGGGG
GGGEAGAVAPYGYTRPPQGLAGQESDFTAPDVWYPGGMVSRVPYPSPTCVKSEMGPW
MDSYSGPYGDMRLETARDHVLPIDYYFPPQKTCLICGDEASGCHYGALTCGSCKVFFKR
AAEGFFRMNKLKESSDTNPKPYCMAAPMGLTENNRNRKKSYRETNLKAVSWPLNHTG
KQKYLCASRNDCTIDKFRRKNCPSCRLRKCYEAGMTLGAAVVVSERILRVFGVSEWLP
AR-v4 (AR1/2/3/2b, AR5)
(SEQ ID NO: 9)
MEVQLGLGRVYPRPPSKTYRGAFQNLFQSVREVIQNPGPRHPEAASAAPPGASLLLLQQ
QQQQQQQQQQQQQQQQQQQQQETSPRQQQQQQGEDGSPQAHRRGPTGYLVLDEEQQ
PSQPQSALECHPERGCVPEPGAAVAASKGLPQQLPAPPDEDDSAAPSTLSLLGPTFPGLSS
CSADLKDILSEASTMQLLQQQQQEAVSEGSSSGRAREASGAPTSSKDNYLGGTSTISDNA
KELCKAVSVSMGLGVEALEHLSPGEQLRGDCMYAPLLGVPPAVRPTPCAPLAECKGSL
LDDSAGKSTEDTAEYSPFKGGYTKGLEGESLGCSGSAAAGSSGTLELPSTLSLYKSGALD
EAAAYQSRDYYNFPLALAGPPPPPPPPHPHARIKLENPLDYGSAWAAAAAQCRYGDLAS
LHGAGAAGPGSGSPSAAASSSWHTLFTAEEGQLYGPCGGGGGGGGGGGGGGGGGGGG
GGGEAGAVAPYGYTRPPQGLAGQESDFTAPDVWYPGGMVSRVPYPSPTCVKSEMGPW
MDSYSGPYGDMRLETARDHVLPIDYYFPPQKTCLICGDEASGCHYGALTCGSCKVFFKR
AAEGKQKYLCASRNDCTIDKFRRKNCPSCRLRKCYEAGMTLGGFFRMNKLKESSDTNP
KPYCMAAPMGLTENNRNRKKSYRETNLKAVSWPLNHTGKQKYLCASRNDCTIDKFRR
KNCPSCRLRKCYEAGMTLGAAVVVSERILRVFGVSEWLP
AR-v5
(SEQ ID NO: 10)
MEVQLGLGRVYPRPPSKTYRGAFQNLFQSVREVIQNPGPRHPEAASAAPPGASLLLLQQ
QQQQQQQQQQQQQQQQQQQQQETSPRQQQQQQGEDGSPQAHRRGPTGYLVLDEEQQ
PSQPQSALECHPERGCVPEPGAAVAASKGLPQQLPAPPDEDDSAAPSTLSLLGPTFPGLSS
CSADLKDILSEASTMQLLQQQQQEAVSEGSSSGRAREASGAPTSSKDNYLGGTSTISDNA
KELCKAVSVSMGLGVEALEHLSPGEQLRGDCMYAPLLGVPPAVRPTPCAPLAECKGSL
LDDSAGKSTEDTAEYSPFKGGYTKGLEGESLGCSGSAAAGSSGTLELPSTLSLYKSGALD
EAAAYQSRDYYNFPLALAGPPPPPPPPHPHARIKLENPLDYGSAWAAAAAQCRYGDLAS
LHGAGAAGPGSGSPSAAASSSWHTLFTAEEGQLYGPCGGGGGGGGGGGGGGGGGGGG
GGGEAGAVAPYGYTRPPQGLAGQESDFTAPDVWYPGGMVSRVPYPSPTCVKSEMGPW
MDSYSGPYGDMRLETARDHVLPIDYYFPPQKTCLICGDEASGCHYGALTCGSCKVFFKR
AAEGKQKYLCASRNDCTIDKFRRKNCPSCRLRKCYEAGMTLGD
AR-v6
(SEQ ID NO: 11)
MEVQLGLGRVYPRPPSKTYRGAFQNLFQSVREVIQNPGPRHPEAASAAPPGASLLLLQQ
QQQQQQQQQQQQQQQQQQQQQETSPRQQQQQQGEDGSPQAHRRGPTGYLVLDEEQQ
PSQPQSALECHPERGCVPEPGAAVAASKGLPQQLPAPPDEDDSAAPSTLSLLGPTFPGLSS
CSADLKDILSEASTMQLLQQQQQEAVSEGSSSGRAREASGAPTSSKDNYLGGTSTISDNA
KELCKAVSVSMGLGVEALEHLSPGEQLRGDCMYAPLLGVPPAVRPTPCAPLAECKGSL
LDDSAGKSTEDTAEYSPFKGGYTKGLEGESLGCSGSAAAGSSGTLELPSTLSLYKSGALD
EAAAYQSRDYYNFPLALAGPPPPPPPPHPHARIKLENPLDYGSAWAAAAAQCRYGDLAS
LHGAGAAGPGSGSPSAAASSSWHTLFTAEEGQLYGPCGGGGGGGGGGGGGGGGGGGG
GGGEAGAVAPYGYTRPPQGLAGQESDFTAPDVWYPGGMVSRVPYPSPTCVKSEMGPW
MDSYSGPYGDMRLETARDHVLPIDYYFPPQKTCLICGDEASGCHYGALTCGSCKVFFKR
AAEGKQKYLCASRNDCTIDKFRRKNCPSCRLRKCYEAGMTLGAGSRVS
AR-v7 (AR3) (GenBank and NCBI Reference Sequence Accession
Number: NP_001334990.1)
(SEQ ID NO: 2)
MEVQLGLGRVYPRPPSKTYRGAFQNLFQSVREVIQNPGPRHPEAASAAPPGASLLLLQQ
QQQQQQQQQQQQQQQQQQQQQETSPRQQQQQQGEDGSPQAHRRGPTGYLVLDEEQQ
PSQPQSALECHPERGCVPEPGAAVAASKGLPQQLPAPPDEDDSAAPSTLSLLGPTFPGLSS
CSADLKDILSEASTMQLLQQQQQEAVSEGSSSGRAREASGAPTSSKDNYLGGTSTISDNA
KELCKAVSVSMGLGVEALEHLSPGEQLRGDCMYAPLLGVPPAVRPTPCAPLAECKGSL
LDDSAGKSTEDTAEYSPFKGGYTKGLEGESLGCSGSAAAGSSGTLELPSTLSLYKSGALD
EAAAYQSRDYYNFPLALAGPPPPPPPPHPHARIKLENPLDYGSAWAAAAAQCRYGDLAS
LHGAGAAGPGSGSPSAAASSSWHTLFTAEEGQLYGPCGGGGGGGGGGGGGGGGGGGG
GGGEAGAVAPYGYTRPPQGLAGQESDFTAPDVWYPGGMVSRVPYPSPTCVKSEMGPW
MDSYSGPYGDMRLETARDHVLPIDYYFPPQKTCLICGDEASGCHYGALTCGSCKVFFKR
AAEGKQKYLCASRNDCTIDKFRRKNCPSCRLRKCYEAGMTLGEKFRVGNCKHLKMTR
P
AR-v8
(SEQ ID NO: 12)
MEVQLGLGRVYPRPPSKTYRGAFQNLFQSVREVIQNPGPRHPEAASAAPPGASLLLLQQ
QQQQQQQQQQQQQQQQQQQQQETSPRQQQQQQGEDGSPQAHRRGPTGYLVLDEEQQ
PSQPQSALECHPERGCVPEPGAAVAASKGLPQQLPAPPDEDDSAAPSTLSLLGPTFPGLSS
CSADLKDILSEASTMQLLQQQQQEAVSEGSSSGRAREASGAPTSSKDNYLGGTSTISDNA
KELCKAVSVSMGLGVEALEHLSPGEQLRGDCMYAPLLGVPPAVRPTPCAPLAECKGSL
LDDSAGKSTEDTAEYSPFKGGYTKGLEGESLGCSGSAAAGSSGTLELPSTLSLYKSGALD
EAAAYQSRDYYNFPLALAGPPPPPPPPHPHARIKLENPLDYGSAWAAAAAQCRYGDLAS
LHGAGAAGPGSGSPSAAASSSWHTLFTAEEGQLYGPCGGGGGGGGGGGGGGGGGGGG
GGGEAGAVAPYGYTRPPQGLAGQESDFTAPDVWYPGGMVSRVPYPSPTCVKSEMGPW
MDSYSGPYGDMRLETARDHVLPIDYYFPPQKTCLICGDEASGCHYGALTCGSCKVFFKR
AAEGKQKYLCASRNDCTIDKFRRKNCPSCRLRKCYEAGMTLGGFDNLCELSS
AR-v9
(SEQ ID NO: 13)
MEVQLGLGRVYPRPPSKTYRGAFQNLFQSVREVIQNPGPRHPEAASAAPPGASLLLLQQ
QQQQQQQQQQQQQQQQQQQQQETSPRQQQQQQGEDGSPQAHRRGPTGYLVLDEEQQ
PSQPQSALECHPERGCVPEPGAAVAASKGLPQQLPAPPDEDDSAAPSTLSLLGPTFPGLSS
CSADLKDILSEASTMQLLQQQQQEAVSEGSSSGRAREASGAPTSSKDNYLGGTSTISDNA
KELCKAVSVSMGLGVEALEHLSPGEQLRGDCMYAPLLGVPPAVRPTPCAPLAECKGSL
LDDSAGKSTEDTAEYSPFKGGYTKGLEGESLGCSGSAAAGSSGTLELPSTLSLYKSGALD
EAAAYQSRDYYNFPLALAGPPPPPPPPHPHARIKLENPLDYGSAWAAAAAQCRYGDLAS
LHGAGAAGPGSGSPSAAASSSWHTLFTAEEGQLYGPCGGGGGGGGGGGGGGGGGGGG
GGGEAGAVAPYGYTRPPQGLAGQESDFTAPDVWYPGGMVSRVPYPSPTCVKSEMGPW
MDSYSGPYGDMRLETARDHVLPIDYYFPPQKTCLICGDEASGCHYGALTCGSCKVFFKR
AAEGKQKYLCASRNDCTIDKFRRKNCPSCRLRKCYEAGMTLGDNLPEQAAFWRHLHIF
WDHVVKK
AR-v10
(SEQ ID NO: 14)
MEVQLGLGRVYPRPPSKTYRGAFQNLFQSVREVIQNPGPRHPEAASAAPPGASLLLLQQ
QQQQQQQQQQQQQQQQQQQQQETSPRQQQQQQGEDGSPQAHRRGPTGYLVLDEEQQ
PSQPQSALECHPERGCVPEPGAAVAASKGLPQQLPAPPDEDDSAAPSTLSLLGPTFPGLSS
CSADLKDILSEASTMQLLQQQQQEAVSEGSSSGRAREASGAPTSSKDNYLGGTSTISDNA
KELCKAVSVSMGLGVEALEHLSPGEQLRGDCMYAPLLGVPPAVRPTPCAPLAECKGSL
LDDSAGKSTEDTAEYSPFKGGYTKGLEGESLGCSGSAAAGSSGTLELPSTLSLYKSGALD
EAAAYQSRDYYNFPLALAGPPPPPPPPHPHARIKLENPLDYGSAWAAAAAQCRYGDLAS
LHGAGAAGPGSGSPSAAASSSWHTLFTAEEGQLYGPCGGGGGGGGGGGGGGGGGGGG
GGGEAGAVAPYGYTRPPQGLAGQESDFTAPDVWYPGGMVSRVPYPSPTCVKSEMGPW
MDSYSGPYGDMRLETARDHVLPIDYYFPPQKTCLICGDEASGCHYGALTCGSCKVFFKR
AAEGKQKYLCASRNDCTIDKFRRKNCPSCRLRKCYEAGMTPSSGTNSVFLPHRDVVRT
GCRSNSGYHSCSCEYHDYCFL
AR-v11
(SEQ ID NO: 15)
MEVQLGLGRVYPRPPSKTYRGAFQNLFQSVREVIQNPGPRHPEAASAAPPGASLLLLQQ
QQQQQQQQQQQQQQQQQQQQQETSPRQQQQQQGEDGSPQAHRRGPTGYLVLDEEQQ
PSQPQSALECHPERGCVPEPGAAVAASKGLPQQLPAPPDEDDSAAPSTLSLLGPTFPGLSS
CSADLKDILSEASTMQLLQQQQQEAVSEGSSSGRAREASGAPTSSKDNYLGGTSTISDNA
KELCKAVSVSMGLGVEALEHLSPGEQLRGDCMYAPLLGVPPAVRPTPCAPLAECKGSL
LDDSAGKSTEDTAEYSPFKGGYTKGLEGESLGCSGSAAAGSSGTLELPSTLSLYKSGALD
EAAAYQSRDYYNFPLALAGPPPPPPPPHPHARIKLENPLDYGSAWAAAAAQCRYGDLAS
LHGAGAAGPGSGSPSAAASSSWHTLFTAEEGQLYGPCGGGGGGGGGGGGGGGGGGGG
GGGEAGAVAPYGYTRPPQGLAGQESDFTAPDVWYPGGMVSRVPYPSPTCVKSEMGPW
MDSYSGPYGDMRLETARDHVLPIDYYFPPQKTCLICGDEASGCHYGALTCGSCKVFFKR
AAEGKQKYLCASRNDCTIDKFRRKNCPSCRLRKCYEAGMTLGGKILFFLFLLLPLSPFSLI
F
AR-v12 (ARV567es)
(SEQ ID NO: 16)
MEVQLGLGRVYPRPPSKTYRGAFQNLFQSVREVIQNPGPRHPEAASAAPPGASLLLLQQ
QQQQQQQQQQQQQQQQQQQQQETSPRQQQQQQGEDGSPQAHRRGPTGYLVLDEEQQ
PSQPQSALECHPERGCVPEPGAAVAASKGLPQQLPAPPDEDDSAAPSTLSLLGPTFPGLSS
CSADLKDILSEASTMQLLQQQQQEAVSEGSSSGRAREASGAPTSSKDNYLGGTSTISDNA
KELCKAVSVSMGLGVEALEHLSPGEQLRGDCMYAPLLGVPPAVRPTPCAPLAECKGSL
LDDSAGKSTEDTAEYSPFKGGYTKGLEGESLGCSGSAAAGSSGTLELPSTLSLYKSGALD
EAAAYQSRDYYNFPLALAGPPPPPPPPHPHARIKLENPLDYGSAWAAAAAQCRYGDLAS
LHGAGAAGPGSGSPSAAASSSWHTLFTAEEGQLYGPCGGGGGGGGGGGGGGGGGGGG
GGGEAGAVAPYGYTRPPQGLAGQESDFTAPDVWYPGGMVSRVPYPSPTCVKSEMGPW
MDSYSGPYGDMRLETARDHVLPIDYYFPPQKTCLICGDEASGCHYGALTCGSCKVFFKR
AAEGKQKYLCASRNDCTIDKFRRKNCPSCRLRKCYEAGMTLGARKLKKLGNLKLQEEG
EASSTTSPTEETTQKLTVSHIEGYECQPIFLNVLEAIEPGVVCAGHDNNQPDSFAALLSSL
NELGERQLVHVVKWAKALPDCERAASVHF
AR-v13
(SEQ ID NO: 17)
MEVQLGLGRVYPRPPSKTYRGAFQNLFQSVREVIQNPGPRHPEAASAAPPGASLLLLQQ
QQQQQQQQQQQQQQQQQQQQQETSPRQQQQQQGEDGSPQAHRRGPTGYLVLDEEQQ
PSQPQSALECHPERGCVPEPGAAVAASKGLPQQLPAPPDEDDSAAPSTLSLLGPTFPGLSS
CSADLKDILSEASTMQLLQQQQQEAVSEGSSSGRAREASGAPTSSKDNYLGGTSTISDNA
KELCKAVSVSMGLGVEALEHLSPGEQLRGDCMYAPLLGVPPAVRPTPCAPLAECKGSL
LDDSAGKSTEDTAEYSPFKGGYTKGLEGESLGCSGSAAAGSSGTLELPSTLSLYKSGALD
EAAAYQSRDYYNFPLALAGPPPPPPPPHPHARIKLENPLDYGSAWAAAAAQCRYGDLAS
LHGAGAAGPGSGSPSAAASSSWHTLFTAEEGQLYGPCGGGGGGGGGGGGGGGGGGGG
GGGEAGAVAPYGYTRPPQGLAGQESDFTAPDVWYPGGMVSRVPYPSPTCVKSEMGPW
MDSYSGPYGDMRLETARDHVLPIDYYFPPQKTCLICGDEASGCHYGALTCGSCKVFFKR
AAEGKQKYLCASRNDCTIDKFRRKNCPSCRLRKCYEAGMTLGARKLKKLGNLKLQEEG
EASSTTSPTEETTQKLTVSHIEGYECQPIFLNVLEAIEPGVVCAGHDNNQPDSFAALLSSL
NELGERQLVHVVKWAKALPGFRNLHVDDQMAVIQYSWMGLMVFAMGWRSFTNVNS
RMLYFAPDLVFNEYRMHKSRMYSQCVRMRHLSQEFGWLQITPQEFLCMKALLLFSINH
T
AR-v14
(SEQ ID NO: 18)
MEVQLGLGRVYPRPPSKTYRGAFQNLFQSVREVIQNPGPRHPEAASAAPPGASLLLLQQ
QQQQQQQQQQQQQQQQQQQQQETSPRQQQQQQGEDGSPQAHRRGPTGYLVLDEEQQ
PSQPQSALECHPERGCVPEPGAAVAASKGLPQQLPAPPDEDDSAAPSTLSLLGPTFPGLSS
CSADLKDILSEASTMQLLQQQQQEAVSEGSSSGRAREASGAPTSSKDNYLGGTSTISDNA
KELCKAVSVSMGLGVEALEHLSPGEQLRGDCMYAPLLGVPPAVRPTPCAPLAECKGSL
LDDSAGKSTEDTAEYSPFKGGYTKGLEGESLGCSGSAAAGSSGTLELPSTLSLYKSGALD
EAAAYQSRDYYNFPLALAGPPPPPPPPHPHARIKLENPLDYGSAWAAAAAQCRYGDLAS
LHGAGAAGPGSGSPSAAASSSWHTLFTAEEGQLYGPCGGGGGGGGGGGGGGGGGGGG
GGGEAGAVAPYGYTRPPQGLAGQESDFTAPDVWYPGGMVSRVPYPSPTCVKSEMGPW
MDSYSGPYGDMRLETARDHVLPIDYYFPPQKTCLICGDEASGCHYGALTCGSCKVFFKR
AAEGKQKYLCASRNDCTIDKFRRKNCPSCRLRKCYEAGMTLGARKLKKLGNLKLQEEG
EASSTTSPTEETTQKLTVSHIEGYECQPIFLNVLEAIEPGVVCAGHDNNQPDSFAALLSSL
NELGERQLVHVVKWAKALPGFRNLHVDDQMAVIQYSWMGLMVFAMGWRSFTNVNS
RMLYFAPDLVFNEYRMHKSRMYSQCVRMRHLSQEFGWLQITPQEFLCMKALLLFSIIPV
DGLKNQKFFDELRMNYIKELDRIIACKRKNPTSCSRRFYQLTKLLDSVQPITPDAMYL
AR-v15
(SEQ ID NO: 19)
MEVQLGLGRVYPRPPSKTYRGAFQNLFQSVREVIQNPGPRHPEAASAAPPGASLLLLQQ
QQQQQQQQQQQQQQQQQQQQQETSPRQQQQQQGEDGSPQAHRRGPTGYLVLDEEQQ
PSQPQSALECHPERGCVPEPGAAVAASKGLPQQLPAPPDEDDSAAPSTLSLLGPTFPGLSS
CSADLKDILSEASTMQLLQQQQQEAVSEGSSSGRAREASGAPTSSKDNYLGGTSTISDNA
KELCKAVSVSMGLGVEALEHLSPGEQLRGDCMYAPLLGVPPAVRPTPCAPLAECKGSL
LDDSAGKSTEDTAEYSPFKGGYTKGLEGESLGCSGSAAAGSSGTLELPSTLSLYKSGALD
EAAAYQSRDYYNFPLALAGPPPPPPPPHPHARIKLENPLDYGSAWAAAAAQCRYGDLAS
LHGAGAAGPGSGSPSAAASSSWHTLFTAEEGQLYGPCGGGGGGGGGGGGGGGGGGGG
GGGEAGAVAPYGYTRPPQGLAGQESDFTAPDVWYPGGMVSRVPYPSPTCVKSEMGPW
MDSYSGPYGDMRLETARDHVLPIDYYFPPQKTCLICGDEASGCHYGALTCGSCKVFFKR
AAEGKQKYLCASRNDCTIDKFRRKNCPSCRLRKCYEAGMTLGARKLKKLGNLKLQEEG
EASSTTSPTEETTQKLTVSHIEGYECQPIFLNVLEAIEPGVVCAGHDNNQPDSFAALLSSL
NELGERQLVHVVKWAKALPGFRNLHVDDQMAVIQYSWMGLMVFAMGWRSFTNVNS
RMLYFAPDLVFNEYRMHKSRMYSQCVRMRHLSQEFGWLQITPQEFLCMKALLLFSISP
VKEQRDKKSRGHDTLYFTSSRQMNVESIKTQWN
AR-v16
(SEQ ID NO: 20)
MEVQLGLGRVYPRPPSKTYRGAFQNLFQSVREVIQNPGPRHPEAASAAPPGASLLLLQQ
QQQQQQQQQQQQQQQQQQQQQETSPRQQQQQQGEDGSPQAHRRGPTGYLVLDEEQQ
PSQPQSALECHPERGCVPEPGAAVAASKGLPQQLPAPPDEDDSAAPSTLSLLGPTFPGLSS
CSADLKDILSEASTMQLLQQQQQEAVSEGSSSGRAREASGAPTSSKDNYLGGTSTISDNA
KELCKAVSVSMGLGVEALEHLSPGEQLRGDCMYAPLLGVPPAVRPTPCAPLAECKGSL
LDDSAGKSTEDTAEYSPFKGGYTKGLEGESLGCSGSAAAGSSGTLELPSTLSLYKSGALD
EAAAYQSRDYYNFPLALAGPPPPPPPPHPHARIKLENPLDYGSAWAAAAAQCRYGDLAS
LHGAGAAGPGSGSPSAAASSSWHTLFTAEEGQLYGPCGGGGGGGGGGGGGGGGGGGG
GGGEAGAVAPYGYTRPPQGLAGQESDFTAPDVWYPGGMVSRVPYPSPTCVKSEMGPW
MDSYSGPYGDMRLETARDHVLPIDYYFPPQKTCLICGDEASGCHYGALTCGSCKVFFKR
AAEGKQKYLCASRNDCTIDKFRRKNCPSCRLRKCYEAGMTLGARKLKKLGNLKLQEEG
EASSTTSPTEETTQKLTVSHIEGYECQPIFLNVLEAIEPGVVCAGHDNNQPDSFAALLSSL
NELGERQLVHVVKWAKALPGFRNLHVDDQMAVIQYSWMGLMVFAMGWRSFTNVNS
RMLYFAPDLVFNEYRMHKSRMYSQCVRMRHLSQEFGWLQITPQEFLCMKALLLFSIIPV
DGLKNQKFFDELRMNYIKELDRIIACKRKNPTSCSRRFYQLTKLLDSVQPIARELHQFTF
DLLIKSHMITPDAMYL
AR-v18
(SEQ ID NO: 21)
MEVQLGLGRVYPRPPSKTYRGAFQNLFQSVREVIQNPGPRHPEAASAAPPGASLLLLQQ
QQQQQQQQQQQQQQQQQQQQQETSPRQQQQQQGEDGSPQAHRRGPTGYLVLDEEQQ
PSQPQSALECHPERGCVPEPGAAVAASKGLPQQLPAPPDEDDSAAPSTLSLLGPTFPGLSS
CSADLKDILSEASTMQLLQQQQQEAVSEGSSSGRAREASGAPTSSKDNYLGGTSTISDNA
KELCKAVSVSMGLGVEALEHLSPGEQLRGDCMYAPLLGVPPAVRPTPCAPLAECKGSL
LDDSAGKSTEDTAEYSPFKGGYTKGLEGESLGCSGSAAAGSSGTLELPSTLSLYKSGALD
EAAAYQSRDYYNFPLALAGPPPPPPPPHPHARIKLENPLDYGSAWAAAAAQCRYGDLAS
LHGAGAAGPGSGSPSAAASSSWHTLFTAEEGQLYGPCGGGGGGGGGGGGGGGGGGGG
GGGEAGAVAPYGYTRPPQGLAGQESDFTAPDVWYPGGMVSRVPYPSPTCVKSEMGPW
MDSYSGPYGDMRLETARDHVLPIDYYFPPQKTCLICGDEASGCHYGALTCGSCKVFFKR
AAEGKQKYLCASRNDCTIDKFRRKNCPSCRLRKCYEAGMTLGARKLKKLGNLKLQEEG
EASSTTSPTEETTQKLTVSHIEGYECQPIFLNVLEAIEPGVVCAGHDNNQPDSFAALLSSL
NELGERQLVHVVKWAKALPGFRNLHVDDQMAVIQYSWMGLMVFAMGWRSFTNVNS
RMLYFAPDLVFNEYRMHKSRMYSQCVRMRHLSQEFGWLQITPQEFLCMKALLLFSISSR
SHLMPCTCERGCSFVLEALSEQTRHLD
AR8 (GenBank and NCBI Reference Sequence Accession
Number: NP_001334993.1)
(SEQ ID NO: 22)
MEVQLGLGRVYPRPPSKTYRGAFQNLFQSVREVIQNPGPRHPEAASAAPPGASLLLLQQ
QQQQQQQQQQQQQQQQQQQQQETSPRQQQQQQGEDGSPQAHRRGPTGYLVLDEEQQ
PSQPQSALECHPERGCVPEPGAAVAASKGLPQQLPAPPDEDDSAAPSTLSLLGPTFPGLSS
CSADLKDILSEASTMQLLQQQQQEAVSEGSSSGRAREASGAPTSSKDNYLGGTSTISDNA
KELCKAVSVSMGLGVEALEHLSPGEQLRGDCMYAPLLGVPPAVRPTPCAPLAECKGSL
LDDSAGKSTEDTAEYSPFKGGYTKGLEGESLGCSGSAAAGSSGTLELPSTLSLYKSGALD
EAAAYQSRDYYNFPLALAGPPPPPPPPHPHARIKLENPLDYGSAWAAAAAQCRYGDLAS
LHGAGAAGPGSGSPSAAASSSWHTLFTAEEGQLYGPCGGGGGGGGGGGGGGGGGGGG
GGGEAGAVAPYGYTRPPQGLAGQESDFTAPDVWYPGGMVSRVPYPSPTCVKSEMGPW
MDSYSGPYGDMRNTRRKRLWKLIIRSINSCICSPRETEVPVRQQK
AR45 (GenBank and NCBI Reference Sequence Accession
Number: NP_001011645.1)
(SEQ ID NO: 23)
MILWLHSLETARDHVLPIDYYFPPQKTCLICGDEASGCHYGALTCGSCKVFFKRAAEGK
QKYLCASRNDCTIDKFRRKNCPSCRLRKCYEAGMTLGARKLKKLGNLKLQEEGEASST
TSPTEETTQKLTVSHIEGYECQPIFLNVLEAIEPGVVCAGHDNNQPDSFAALLSSLNELGE
RQLVHVVKWAKALPGFRNLHVDDQMAVIQYSWMGLMVFAMGWRSFTNVNSRMLYF
APDLVFNEYRMHKSRMYSQCVRMRHLSQEFGWLQITPQEFLCMKALLLFSIIPVDGLKN
QKFFDELRMNYIKELDRIIACKRKNPTSCSRRFYQLTKLLDSVQPIARELHQFTFDLLIKS
HMVSVDFPEMMAEIISVQVPKILSGKVKPIYFHTQ

In certain embodiments, an androgen receptor, or variant thereof, is expressed in a cell. The cell may be obtained from any source (e.g., human) and in the case of prostate cancer cells, the source may specifically be a male human. In certain embodiments, the cell is a prostate gland cell. In certain embodiments, the cell is a prostate cancer cell. In certain embodiments, the cell is a metastatic prostate cancer cell. In certain embodiments, the cell is a castration-resistant prostate cancer cell. In certain embodiments, the prostate cancer cells are maintained in vitro. In certain embodiments, the prostate cancer cells are maintained in vivo. In certain embodiments, in vivo expression is the expression of an androgen receptor or fragment thereof in a cell (e.g., mammalian cells, yeast cells, bacterial cells, etc.). In certain embodiments, the cell is mammalian cell (e.g., human, murine, etc.). In certain embodiments, the mammalian cell is a human cell. In certain embodiments, the human cell is a prostate gland cell, e.g., epithelial prostate gland cell or stromal prostate gland cell. In certain embodiments, the prostate gland cell is an epithelial prostate gland cell, e.g., luminal secretory cells, basal cells, or rare neuroendocrine cells. In certain embodiments, the prostate gland cell is a stromal prostate gland cell, e.g., smooth muscle cell or fibroblast. In certain embodiments, the prostate cancer cells are maintained ex vivo (e.g., in an external environment with minimal alteration of natural conditions). In certain embodiments, the prostate cancer cell is a LNCaP cell.

In certain embodiments, the human cell is derived from a subject with prostate cancer. The subject may be a male human. In certain embodiments, the human cell is derived from a subject with prostate cancer who has not received treatment in any form, e.g., radiation, chemotherapy, surgical procedure (e.g., castration), or administration of one or more therapeutic agents (e.g., abiraterone acetate, bicalutamide, cabazitaxel, casodex, degarelix, docetaxel, enzalutamide, flutamide, goserelin acetate, jevtana, leuprolide acetate, lupron, lupron depot, lupron depot-ped, mitoxantrone hydrochloride, nilandron, nilutamide, provenge, sipuleucel-T, radium 223 dichloride, taxotere, viadur, xofigo, xtandi, zoladex, zytiga). In certain embodiments, the human cell is derived from a subject with prostate cancer who has received treatment in any form, e.g., radiation, chemotherapy, surgical procedure (e.g., castration), or administration of one or more therapeutic agents (e.g., abiraterone acetate, bicalutamide, cabazitaxel, casodex, degarelix, docetaxel, enzalutamide, flutamide, goserelin acetate, jevtana, leuprolide acetate, lupron, lupron depot, lupron depot-ped, mitoxantrone hydrochloride, nilandron, nilutamide, provenge, sipuleucel-T, radium 223 dichloride, taxotere, viadur, xofigo, xtandi, zoladex, or zytiga). In certain embodiments, the human cell is derived from a subject with prostate cancer who has received treatment in any form and the cancer has developed resistance to any form of treatment. In certain embodiments, the prostate cancer cell is a metastatic prostate cancer cell. In certain embodiments, the prostate cancer cell is a castration-resistant prostate cancer cell. In certain embodiments, the prostate cancer cell is a LNCaP cell or derived from the LNCaP cell line. In certain embodiments, the LNCaP cell is an androgen-sensitive human prostate adenocarcinoma cell. In certain embodiments, the LNCaP cells are derived from the lymph node. In certain embodiments, the castration-resistant prostate cancer cell is a C4-2B PCa cell.

In some embodiments a mammalian nucleic acid sequence, e.g., a human nucleic acid sequence, e.g., a human DNA sequence encoding an androgen receptor (e.g., AR-FL, AR-v7), or variant thereof, may be codon optimized for increased expression in a cell. In certain embodiments, a sequence encoding an androgen receptor, or variant thereof may be codon optimized for increased expression in a prostate cancer cell.

In some embodiments, an androgen receptor, or variant thereof, is provided in a purified form. In some embodiments, an androgen receptor, or variant thereof, is provided in the form of a cell lysate.

In some embodiments, an androgen receptor, or variant thereof, comprises a fusion protein comprising an androgen receptor joined at its N-terminus or C-terminus to a second polypeptide. The second polypeptide can be, for example, an epitope, a selectable protein, an enzyme, or a detection protein. For example, the second polypeptide can be beta-galactosidase, hemagglutinin (HA), green fluorescent protein (GFP), FLAG, Myc, or the like. For example, in certain embodiments a fusion protein comprising an androgen receptor or fragment thereof and a HA tag (e.g., HA-tagged AR). It should be understood that the polypeptide components of a fusion protein may be directly joined to each other or may be joined via a linker peptide. In general, a linker peptide may comprise any amino acid sequence and may be of any length, e.g., between 1 and 100 amino acids in length, e.g., between about 10 and about 25 amino acids in length. Suitable linkers may comprise multiple Gly and/or Ser residues.

Compounds that Modulate Androgen Receptor and its Splice Variants

In some aspects, the screening methods of the present disclosure as described above have been used to identify compounds that can modulate an androgen receptor or fragment thereof. In certain embodiments, the screening methods of the present disclosure as described above identified one or more compounds that can modulate the androgen receptor variant AR-v7. Exemplary compounds are shown in Table 2.

TABLE 2

| | |
|---|---|
| 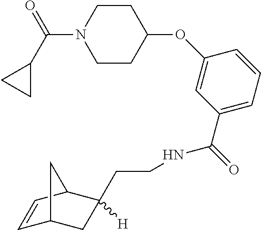 [SMILES: O=C(NCCC1CC2C=CC1C2)c1cccc(OC2CCN(C(=O)C3CC3)CC2)c1] | KI-ARv-01 |
| 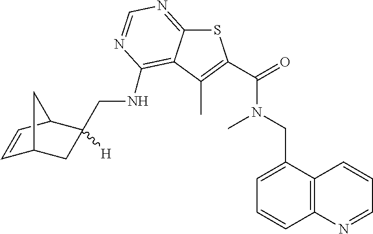 [SMILES: Cc1c(C(=O)N(C)Cc2cccc3ncccc23)sc2ncnc(NCC3CC4C=CC3C4)c12] | KI-ARv-02 |
| 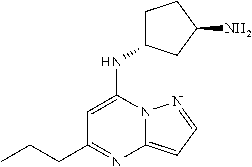 [SMILES: CCCc1cc(N[C@@H]2CC[C@@H](N)C2)n2nccc2n1] | KI-ARv-03 |
| 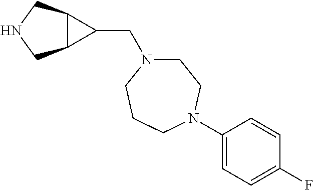 [SMILES: Fc1ccc(N2CCCN(CC3[C@H]4CNC[C@@H]34)CC2)cc1] | KI-ARv-04 |
| 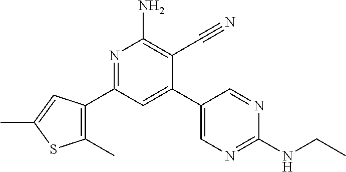 [SMILES: CCNc1ncc(-c2cc(-c3cc(C)sc3C)nc(N)c2C#N)cn1] | KI-ARv-05 |
| 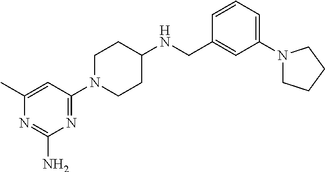 [SMILES: Cc1cc(N2CCC(NCc3cccc(N4CCCC4)c3)CC2)nc(N)n1] | KI-ARv-06 |
| 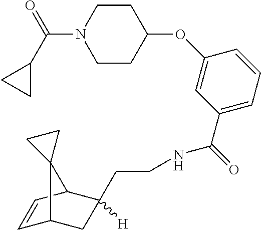 [SMILES: O=C(NCCC1CC2C=CC1C21CC1)c1cccc(OC2CCN(C(=O)C3CC3)CC2)c1] | KI-ARv-07 |
| 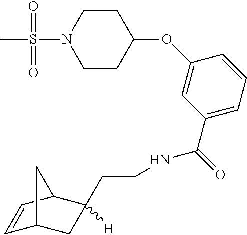 [SMILES: CS(=O)(=O)N1CCC(Oc2cccc(C(=O)NCCC3CC4C=CC3C4)c2)CC1] | KI-ARv-08 |
| 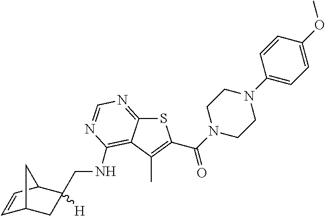 [SMILES: COc1ccc(N2CCN(C(=O)c3sc4ncnc(NCC5CC6C=CC5C6)c4c3C)CC2)cc1] | KI-ARv-09 |

In another aspect, described herein are compounds selected from Table 2, and pharmaceutically acceptable salts, solvates, hydrates, polymorphs, co-crystals, tautomers, stereoisomers, isotopically labeled derivatives, and prodrugs thereof and methods of using the compounds.

In another aspect, the compounds shown in Table 2 modulate the androgen receptor. Thus, the present disclosure also contemplates the treatment of a disease, disorder, or condition which results from aberrant androgen receptor function.

In yet another aspect, a compound selected from those shown in Table 2 is an androgen receptor (e.g., AR-FL, AR-v7) inhibitor. In certain embodiments, a compound selected from those shown in Table 2 is an AR-FL inhibitor. In certain embodiments, a compound selected from those shown in Table 2 is an androgen receptor variant (AR-v) inhibitor. In certain embodiments, a compound selected from those shown in Table 2 is an AR-v7 inhibitor. In certain embodiments, a compound disclosed herein inhibits the androgen receptor by preventing the nuclear translocation of the androgen receptor. In certain embodiments, a compound selected from those shown in Table 2 is an androgen receptor (e.g., AR-FL, AR-v7) antagonist. In certain embodiments, a compound selected from those shown in Table 2 is an AR-FL antagonist. In certain embodiments, a compound selected from those shown in Table 2 is an androgen receptor variant (AR-v) antagonist. In certain embodiments, a compound selected from those shown in Table 2 is an AR-v7 antagonist.

In some aspects, the present disclosure provides a method of modulating the expression of a gene in a cell, the method comprising exposing a cell expressing the androgen receptor (e.g., AR-FL, AR-v7), or variant thereof, to a compound selected from Table 2, or pharmaceutical composition thereof. In certain embodiments, the gene is any gene that may be expressed differently (e.g., overexpressed, underexpressed) in prostate cancer cells. In certain embodiments, the gene is any gene that may be differentially expressed (e.g., overexpressed or underexpressed) in prostate cancer cells compared to cells with a normal prostate gland cell phenotype (e.g., non-cancerous cells). See, e.g., Jariwala et al. (2007) Identification of novel androgen receptor target genes in prostate cancer Mol Cancer, 6: doi:10.1186/1476-4598-6-39. The gene may be underexpressed in prostate cancer cells or metastatic prostate cancer cells. In certain embodiments, the gene is underexpressed in castration-resistant prostate cancer cells. Alternatively, the gene may be overexpressed in prostate cancer cells. The gene may be overexpressed in metastatic or castration-resistant prostate cancer cells. In certain embodiments, the gene that is expressed differentially (e.g., overexpressed or underexpressed) in prostate cancer cells is a gene whose transcription is under direct or indirect control of the androgen receptor or a fragment thereof (e.g., androgen-receptor mediated). In certain embodiments, the gene is overexpressed in prostate cancer cells, such as castration-resistant prostate cancer cells. In certain embodiments, the gene is a human gene wherein the expression of the gene is under direct or indirect control of the androgen receptor or fragment thereof (e.g., androgen-receptor mediated gene expression). The gene may be selected from ACBD6, AKT1, ALG12, AP2A2, AQP12, BAG1, BAZ1B, BRCA1, CARKL, CDK1, CDK2, CDK9, CEP350, CHRM1, CLDN4, COX5B, CRELD2, DACH1, DDT, EFCAB6, FDZ9, FGF8, FOXO1, GAPDH, GNB2L1, GSK3B, GSTT2, HDAC1, HSP90AA1, HTATIP, JUN, KIAA1217, KIF1A, LHPP, LHX4, MAFG, MAGEA11, MAN2B2, MAP3K7IP1, MED1, MRFAP1, MUC6, MYST2, NCOA1, NCOA2, NCOA3, NCOA4, NCOA6, NCOR2, NONO, OAT, PA2G4, PAK6, PATZ1, PIAS2, PMEPA1, PRKCD, PRPF6, PSA, PTEN, PYCRI, QSCN6, RAD9A, RANBP9, RCHY1, RNF14, RNF4, SART3, SIRT1, SLC22A8, SCL22A6, SIRT7, SMAD3, SRC, SRY, STAT3, SVIL, SYNGR1, TGFB1I1, TMF1, TMPRSS2, TRIM68, TRPV1, TRPV3, UBE2I, UXT, WBSCR28, WBSCR27, and ZMIZ1. In certain embodiments, the gene is AKT1.

In some aspects, the present disclosure provides a method of modulating the expression of a gene in a cell, the method comprising contacting a cell expressing the androgen receptor (e.g., AR-FL, AR-v7), or variant thereof, with a compound selected from Table 2, or pharmaceutical composition thereof. In certain embodiments, the gene is any gene that may be expressed differently (e.g., overexpressed, underexpressed) in prostate cancer cells. In certain embodiments, the gene is any gene that may be differentially expressed (e.g., overexpressed or underexpressed) in prostate cancer cells compared to cells with a normal prostate gland cell phenotype (e.g., non-cancerous cells). See, e.g., Jariwala et al. (2007) Identification of novel androgen receptor target genes in prostate cancer Mol Cancer, 6: doi:10.1186/1476-4598-6-39. The gene may be underexpressed in prostate cancer cells or metastatic prostate cancer cells. In certain embodiments, the gene is underexpressed in castration-resistant prostate cancer cells. Alternatively, the gene may be overexpressed in prostate cancer cells. The gene may be overexpressed in metastatic or castration-resistant prostate cancer cells. In certain embodiments, the gene that is expressed differentially (e.g., overexpressed or underexpressed) in prostate cancer cells is a gene whose transcription is under direct or indirect control of the androgen receptor or a fragment thereof (e.g., androgen-receptor mediated). In certain embodiments, the gene is overexpressed in prostate cancer cells, such as castration-resistant prostate cancer cells. In certain embodiments, the gene is a human gene wherein the expression of the gene is under direct or indirect control of the androgen receptor or fragment thereof (e.g., androgen-receptor mediated gene expression). The gene may be selected from ACBD6, AKT1, ALG12, AP2A2, AQP12, BAG1, BAZ1B, BRCA1, CARKL, CDK1, CDK2, CDK9, CEP350, CHRM1, CLDN4, COX5B, CRELD2, DACH1, DDT, EFCAB6, FDZ9, FGF8, FOXO1, GAPDH, GNB2L1, GSK3B, GSTT2, HDAC1, HSP90AA1, HTATIP, JUN, KIAA1217, KIF1A, LHPP, LHX4, MAFG, MAGEA11, MAN2B2, MAP3K7IP1, MED1, MRFAP1, MUC6, MYST2, NCOA1, NCOA2, NCOA3, NCOA4, NCOA6, NCOR2, NONO, OAT, PA2G4, PAK6, PATZ1, PIAS2, PMEPA1, PRKCD, PRPF6, PSA, PTEN, PYCRI, QSCN6, RAD9A, RANBP9, RCHY1, RNF14, RNF4, SART3, SIRT1, SLC22A8, SCL22A6, SIRT7, SMAD3, SRC, SRY, STAT3, SVIL, SYNGR1, TGFB1I1, TMF1, TMPRSS2, TRIM68, TRPV1, TRPV3, UBE2I, UXT, WBSCR28, WBSCR27, and ZMIZ1. In certain embodiments, the gene is AKT1.

In another aspect, the present disclosure contemplates the treatment of a disease, disorder, or condition associated with abnormal androgen receptor-mediated gene expression. In certain embodiments, the compounds shown in Table 2 are capable of modulating expression of a gene, wherein the gene is under direct or indirect control of the androgen receptor or fragment thereof. In certain embodiments, the compounds shown in Table 2 are capable of modulating expression of a gene, wherein the gene is under direct or indirect control of AR-v7. In certain embodiments, the gene is AKT1. AKT1 encodes the enzyme AKT1, i. e., AKT1 is the gene product of AKT1, also known as the RAC-alpha serine/threonine-protein kinase. Increased expression of AKT1 as a result of increased AR activity can lead to increased cell proliferation and differentiation and evasion of apoptosis. This direct AR control of gene expression could be one mechanism that promotes the development and progression of prostate cancer.

Thus, in certain embodiments, provided is a method of modulating the expression of AKT1, comprising administering an effective amount of a compound selected from those shown in Table 2, or pharmaceutically acceptable salt thereof, to a subject.

In another aspect, provided is a method of treating prostate cancer, metastatic prostate cancer, androgen-deprivation resistant prostate cancer, castration-resistant prostate cancer, and the like, the method comprising administering an effective amount of a compound selected from those shown in Table 2, or pharmaceutically acceptable salt thereof, to a subject. In certain embodiments, the method comprises administering a compound of formula

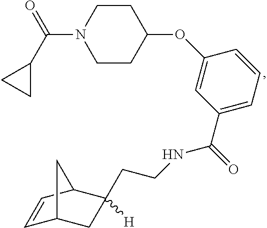 [SMILES: O=C(NCCC1CC2C=CC1C2)c1cccc(OC2CCN(C(=O)C3CC3)CC2)c1]

or a pharmaceutically acceptable salt thereof. In certain embodiments, the method comprises administering a compound of formula

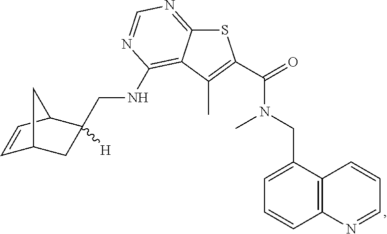 [SMILES: Cc1c(C(=O)N(C)Cc2cccc3ncccc23)sc2ncnc(NCC3CC4C=CC3C4)c12]

or a pharmaceutically acceptable salt thereof.

In another aspect, the present disclosure provides a compound that may be used as a targeting moiety to target an androgen receptor, or variant thereof. In certain embodiments, the compound may be attached to a linker of any composition. The compound may be attached to a linker peptide. In general, a linker peptide may comprise any amino acid sequence and may be of any length, e.g., between 1 and 100 amino acids in length, e.g., between about 10 and about 25 amino acids in length. Suitable linkers may comprise multiple Gly and/or Ser residues. In certain embodiments, the compound may be attached to a linker of any length and composition, wherein the linker is further attached to a molecule of interest (e.g., an agent useful for diagnostic purposes). In certain embodiments, the compound may be attached to a linker of any length and composition, wherein the linker is further attached to a diagnostic agent (e.g., a molecule used to detect a disease or abnormal function in a subject). Thus, the compound may be used to identify the presence of AR-vs in a subject. In certain embodiments, the compound may be attached to a linker of any length and composition, wherein the linker is further attached to a therapeutic agent (e.g., abiraterone acetate, bicalutamide, cabazitaxel, casodex, degarelix, docetaxel, enzalutamide, flutamide, goserelin acetate, jevtana, leuprolide acetate, lupron, lupron depot, lupron depot-ped, mitoxantrone hydrochloride, nilandron, nilutamide, provenge, sipuleucel-T, radium 223 dichloride, taxotere, viadur, xofigo, xtandi, zoladex, or zytiga). Thus, the compound may be used to deliver a therapeutic to a cell expressing an androgen receptor (e.g., AR-v7).

In an embodiment, the present disclosure provides a method for diagnosing a patient, the method comprising administering to the subject a therapeutically effective amount of a compound, or pharmaceutical composition thereof, described herein, wherein the compound is a compound comprising a targeting moiety that targets the androgen receptor.

In an embodiment, the compound being administered or used selectively modulates an androgen receptor (e.g., AR-FL, AR-v7), or variant thereof. When a compound, pharmaceutical composition, method, use, or kit is referred to as “selectively,” “specifically,” or “competitively” modulating a target (e.g., a protein), the compound, pharmaceutical composition, method, use, or kit modulates a particular target (e.g., a target protein) to a greater extent (e.g., not less than 2-fold, not less than 5-fold, not less than 10-fold, not less than 30-fold, not less than 100-fold, not less than 1,000-fold, or not less than 10,000-fold; and/or: not more than 2-fold, not more than 5-fold, not more than 10-fold, not more than 30-fold, not more than 100-fold, not more than 1,000-fold, or not more than 10,000-fold) than another protein.

The present disclosure provides pharmaceutical compositions comprising a compound selected from those listed in Table 2, or a pharmaceutically acceptable salt thereof, and optionally a pharmaceutically acceptable excipient. In certain embodiments, the pharmaceutical composition described herein comprises a compound selected from those listed in Table 2, or a pharmaceutically acceptable salt thereof, and a pharmaceutically acceptable excipient. In an embodiment, the compound described herein is provided in an effective amount in the pharmaceutical composition. In certain embodiments, the pharmaceutical composition comprises a pharmaceutically acceptable excipient and a compound of formula

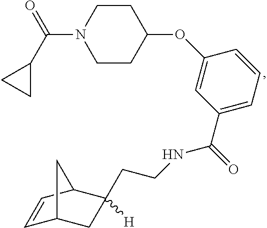 [SMILES: O=C(NCCC1CC2C=CC1C2)c1cccc(OC2CCN(C(=O)C3CC3)CC2)c1]

or a pharmaceutically acceptable salt thereof. In certain embodiments, the pharmaceutical composition comprises a pharmaceutically acceptable excipient and a compound of formula

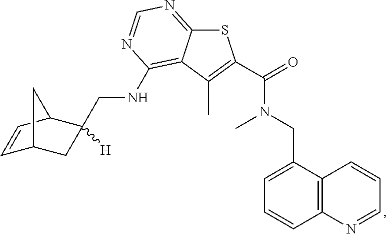 [SMILES: Cc1c(C(=O)N(C)Cc2cccc3ncccc23)sc2ncnc(NCC3CC4C=CC3C4)c12]

or a pharmaceutically acceptable salt thereof.

In certain embodiments, the effective amount is an amount effective for inhibiting the activity of an androgen receptor, or variant thereof, by at least about 10%, at least about 20%, at least about 30%, at least about 40%, at least about 50%, at least about 60%, at least about 70%, at least about 80%, at least about 90%, at least about 95%, or at least about 98%. In certain embodiments, the effective amount is an amount effective for inhibiting the activity of AR-v7 by at least about 10%, at least about 20%, at least about 30%, at least about 40%, at least about 50%, at least about 60%, at least about 70%, at least about 80%, at least about 90%, at least about 95%, or at least about 98%.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition. The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity of a protein kinase in a subject or cell), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both.

The compound or composition can be administered concurrently with, prior to, or subsequent to one or more additional pharmaceutical agents, which are different from the compound or composition and may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, cytotoxic agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is a binder or inhibitor of a protein kinase. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy.

In one aspect, the present disclosure provides compounds for use for treatment of cancer. The compound for use for treatment of prostate cancer can be any compound selected from those listed in Table 2, or a pharmaceutically acceptable salt thereof. These compounds may be used to treat prostate cancer, which can be metastatic or castration-resistant prostate cancer (CRPC). In certain embodiments, the compound for use for treatment of prostate cancer is

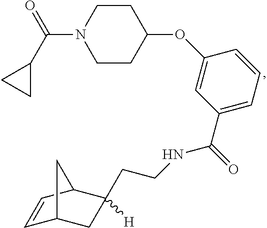 [SMILES: O=C(NCCC1CC2C=CC1C2)c1cccc(OC2CCN(C(=O)C3CC3)CC2)c1]

or a pharmaceutically acceptable salt thereof. In certain embodiments, the compound for use for treatment of prostate cancer is

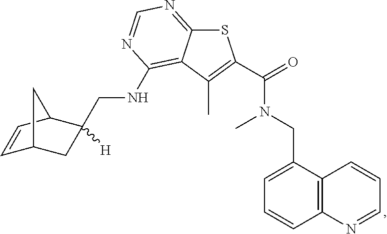 [SMILES: Cc1c(C(=O)N(C)Cc2cccc3ncccc23)sc2ncnc(NCC3CC4C=CC3C4)c12]

or a pharmaceutically acceptable salt thereof. In certain embodiments, the compound for use for treatment of prostate cancer is

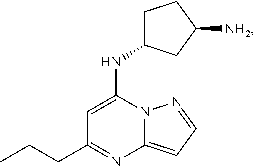 [SMILES: CCCc1cc(N[C@@H]2CC[C@@H](N)C2)n2nccc2n1]

or a pharmaceutically acceptable salt thereof.

### EXAMPLES

In order that the invention described herein may be more fully understood, the following examples are set forth. The examples described in this application are offered to illustrate the methods, compositions, and systems provided herein and are not to be construed in any way as limiting their scope.

### Example 1

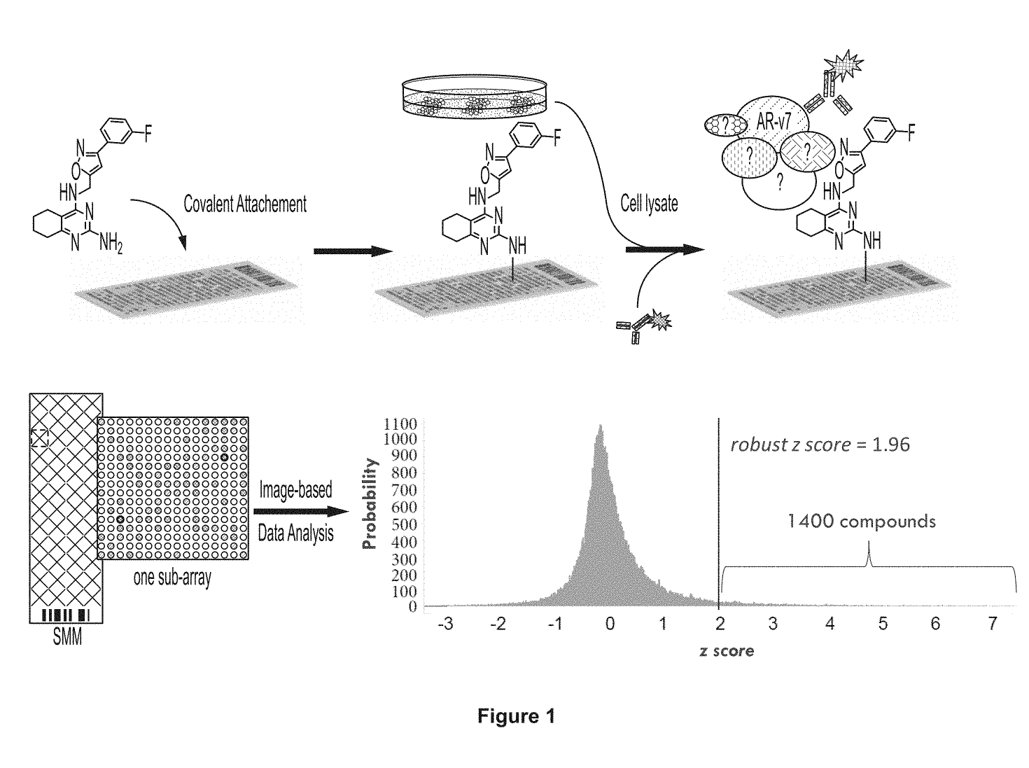

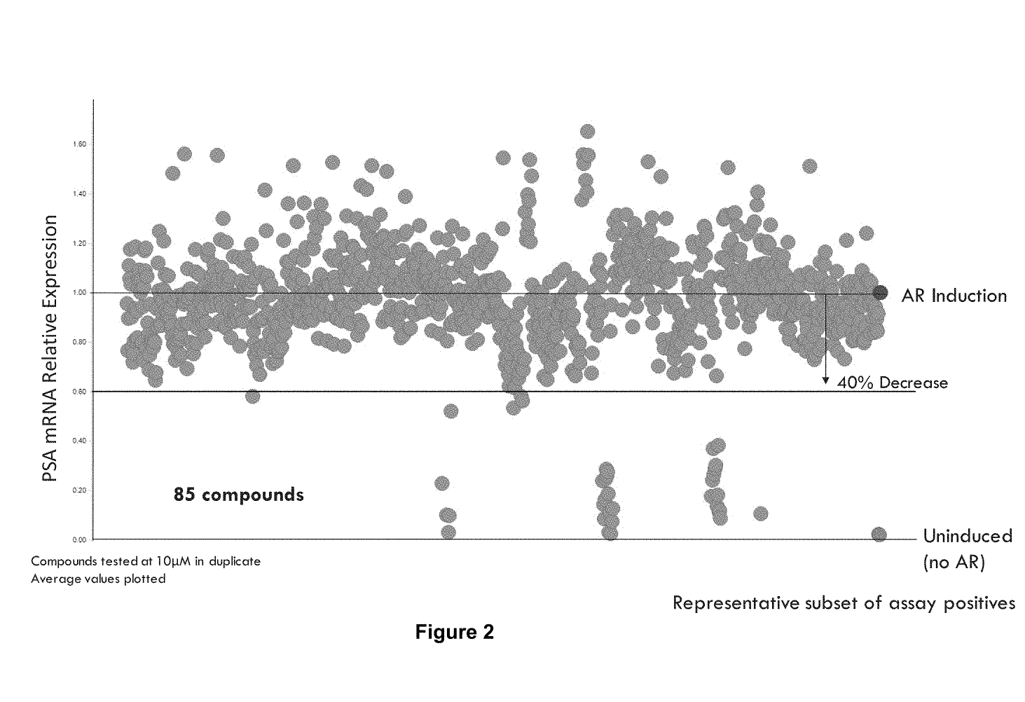

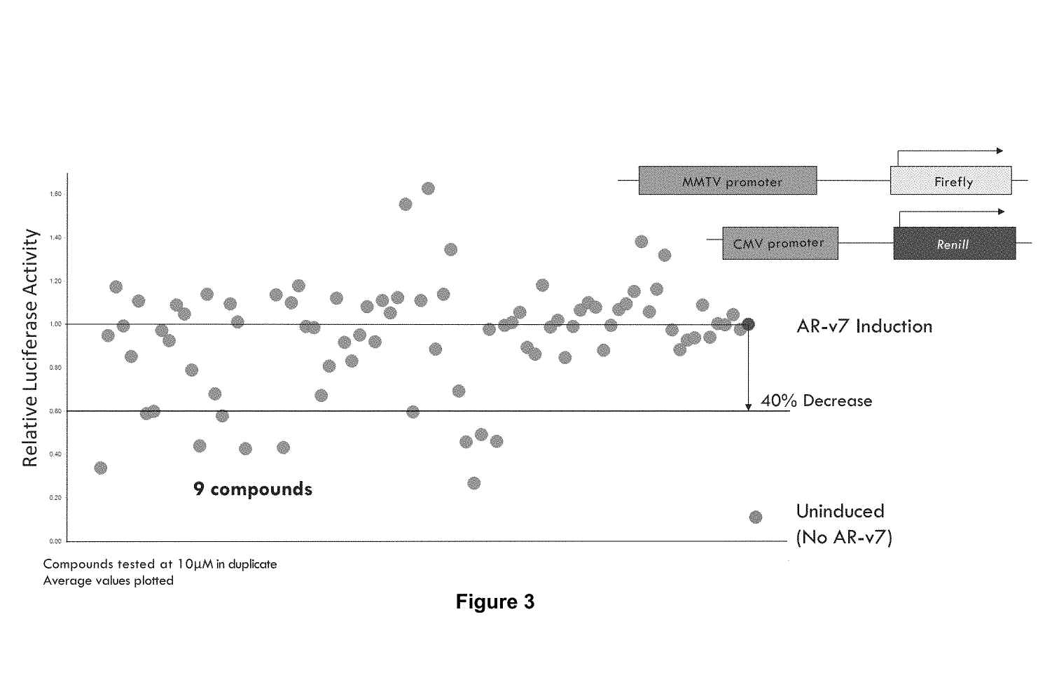

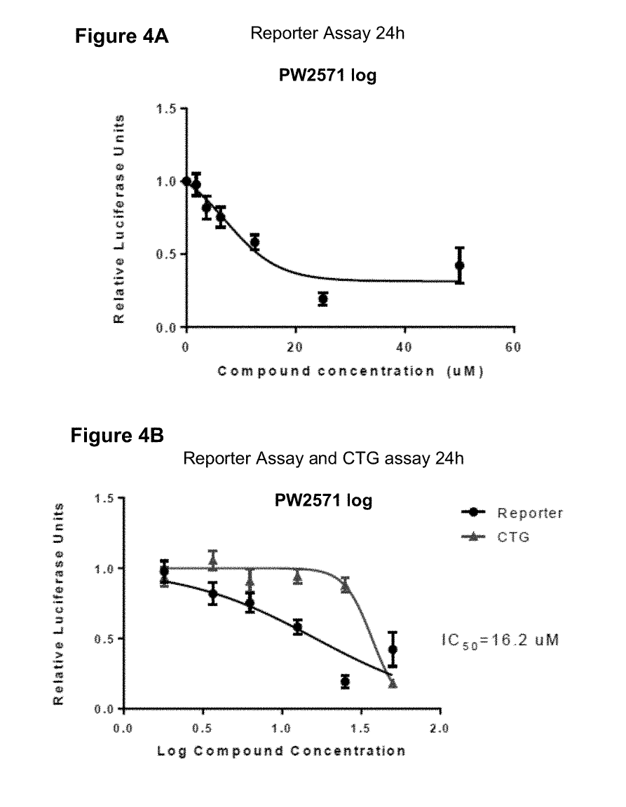

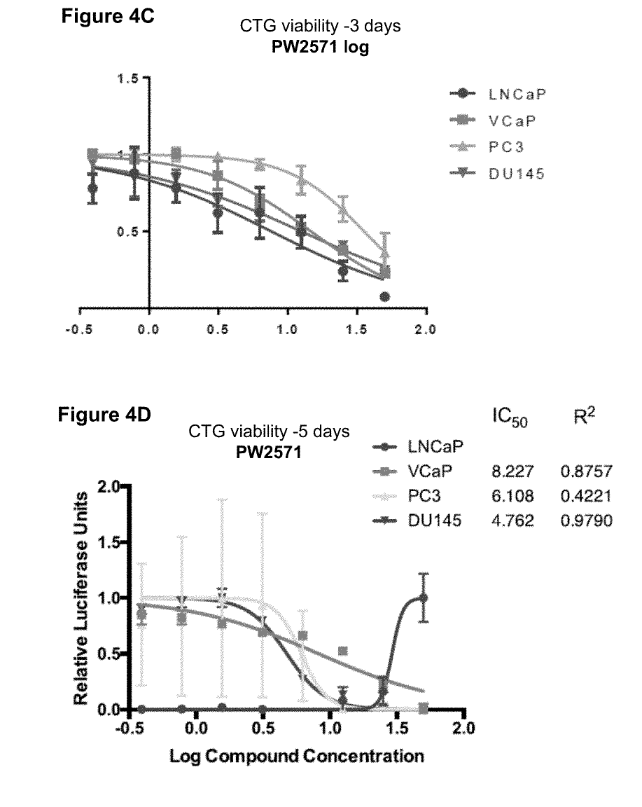

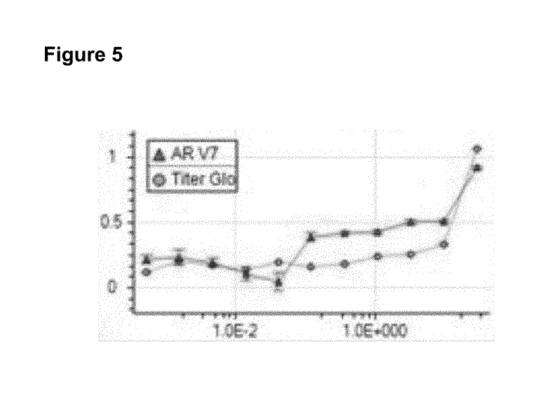

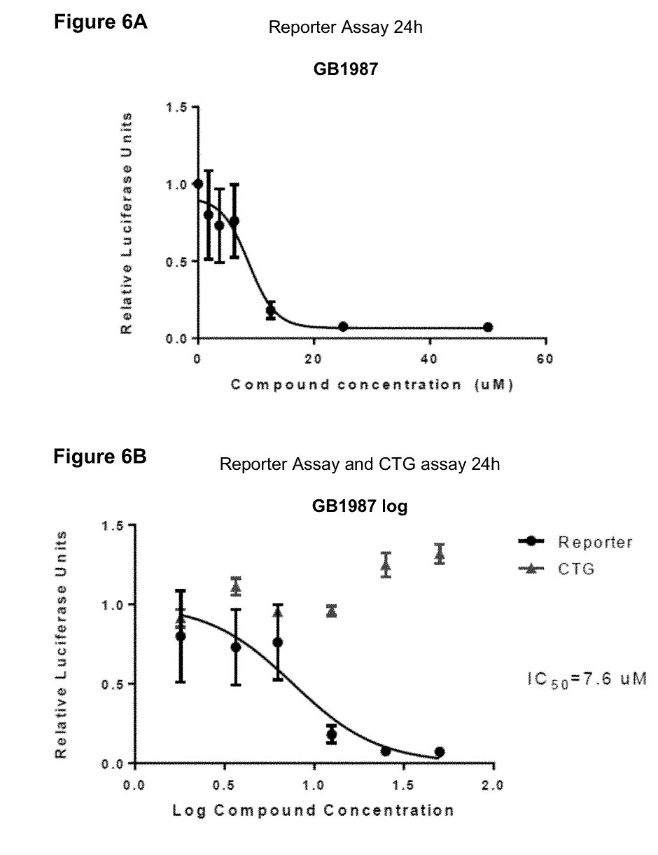

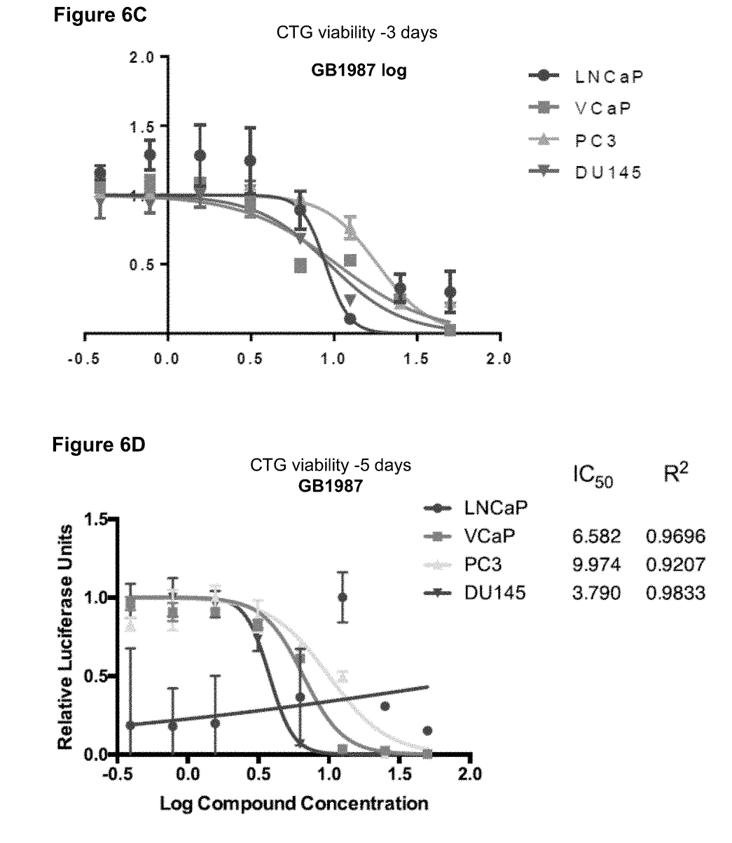

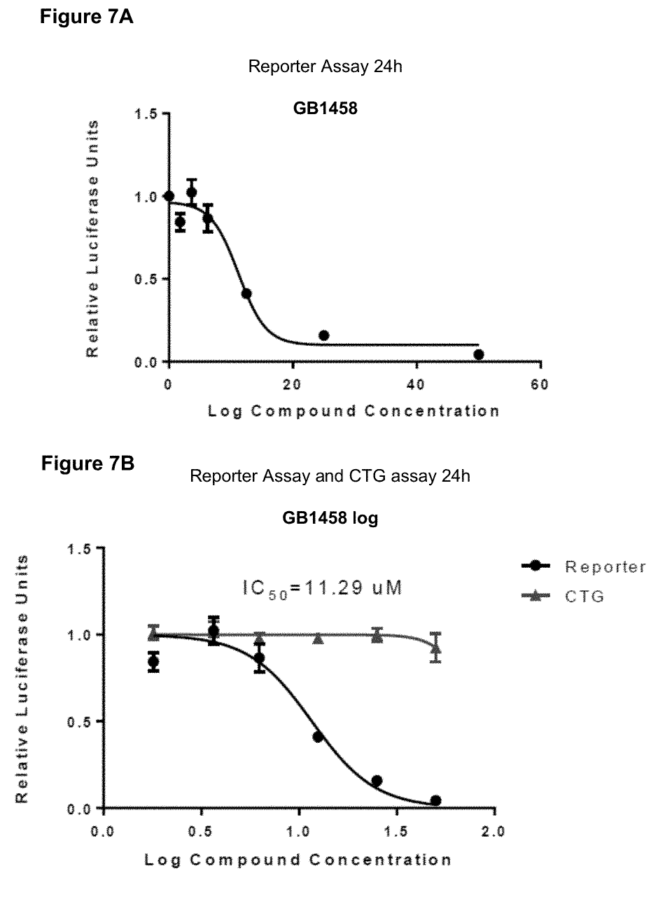

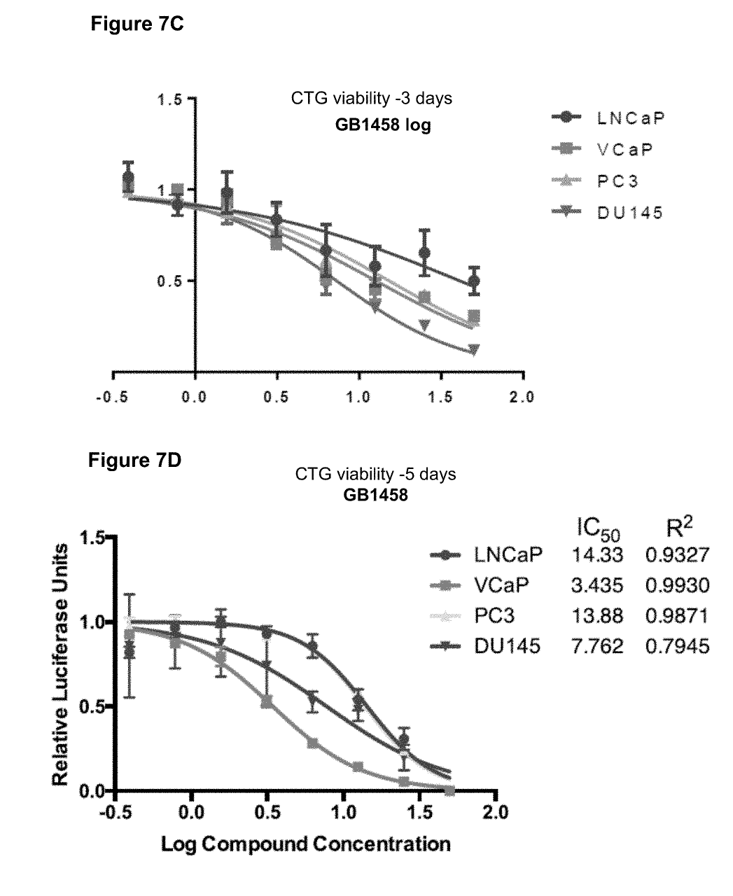

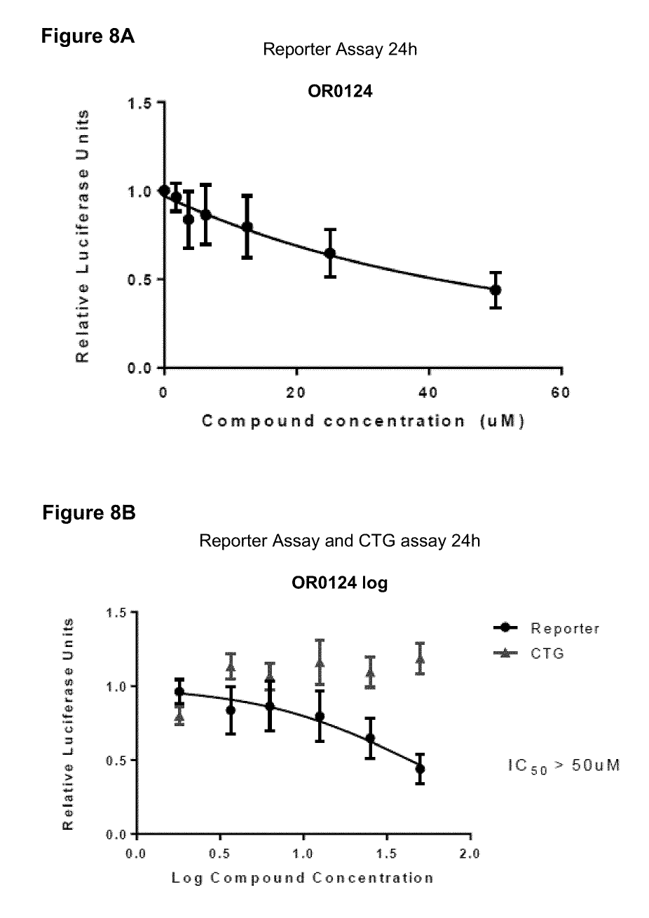

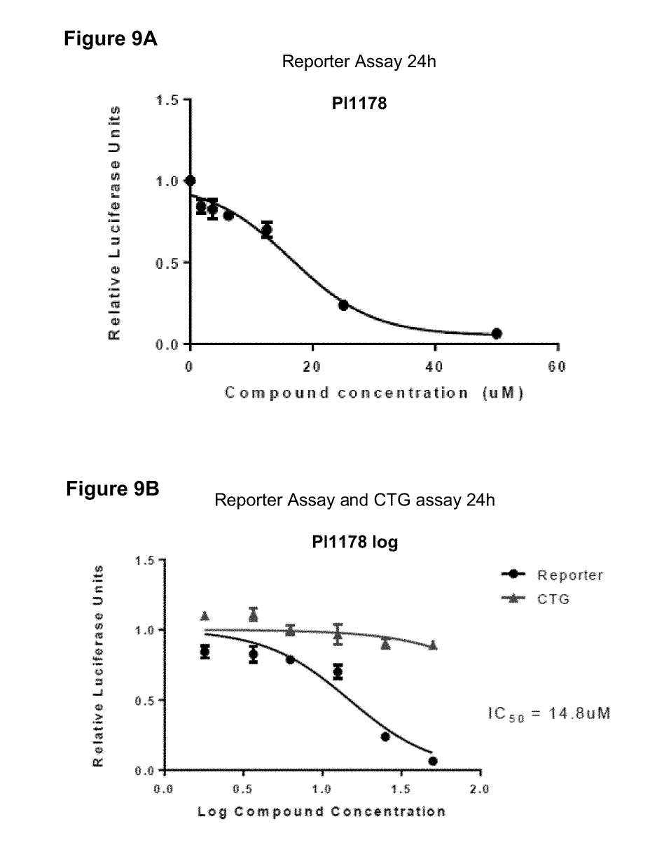

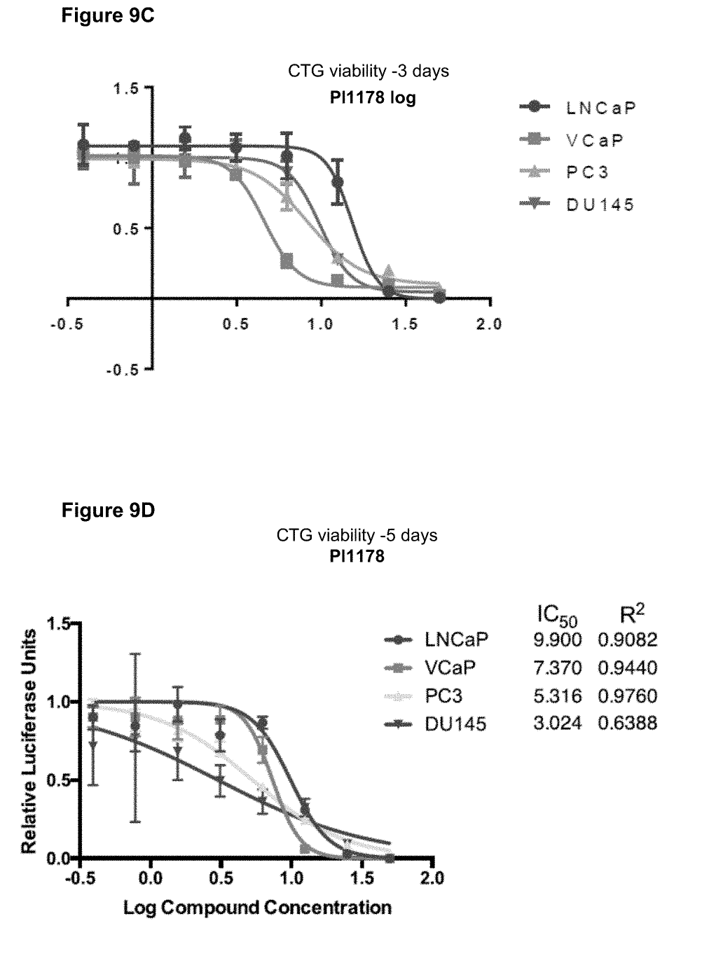

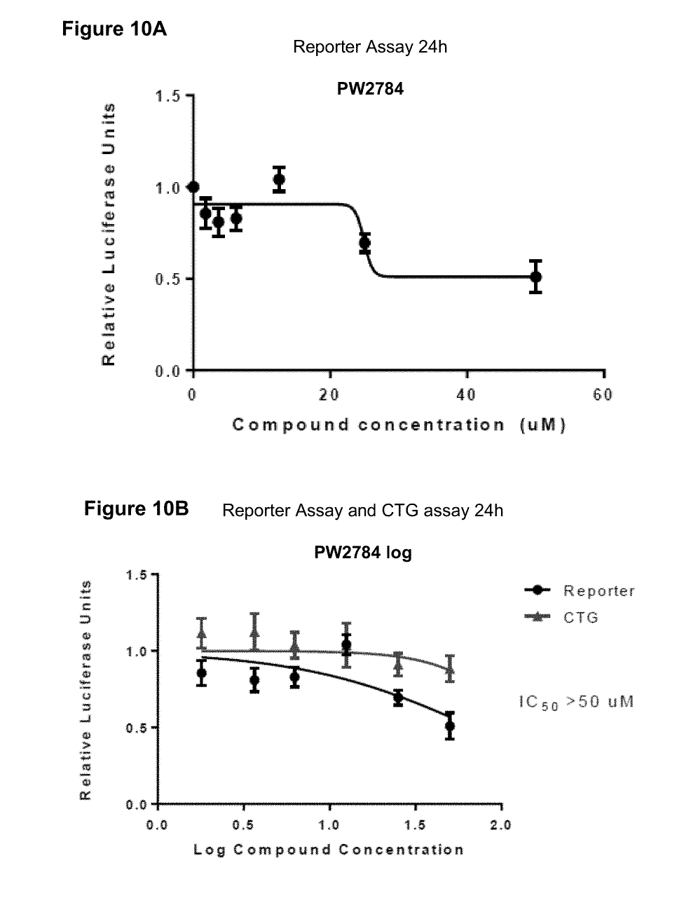

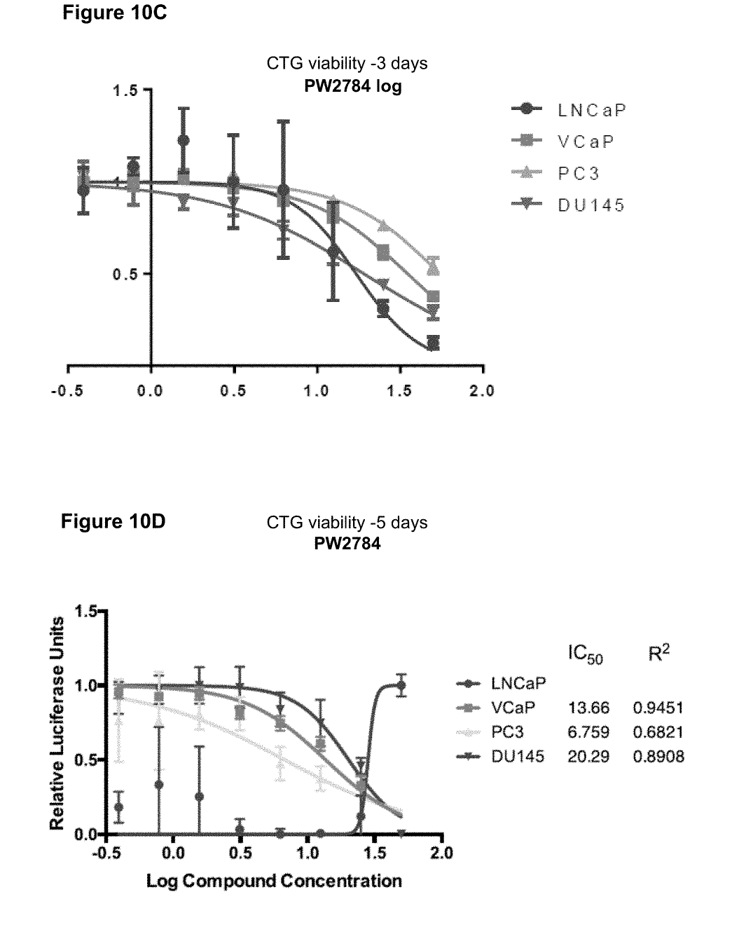

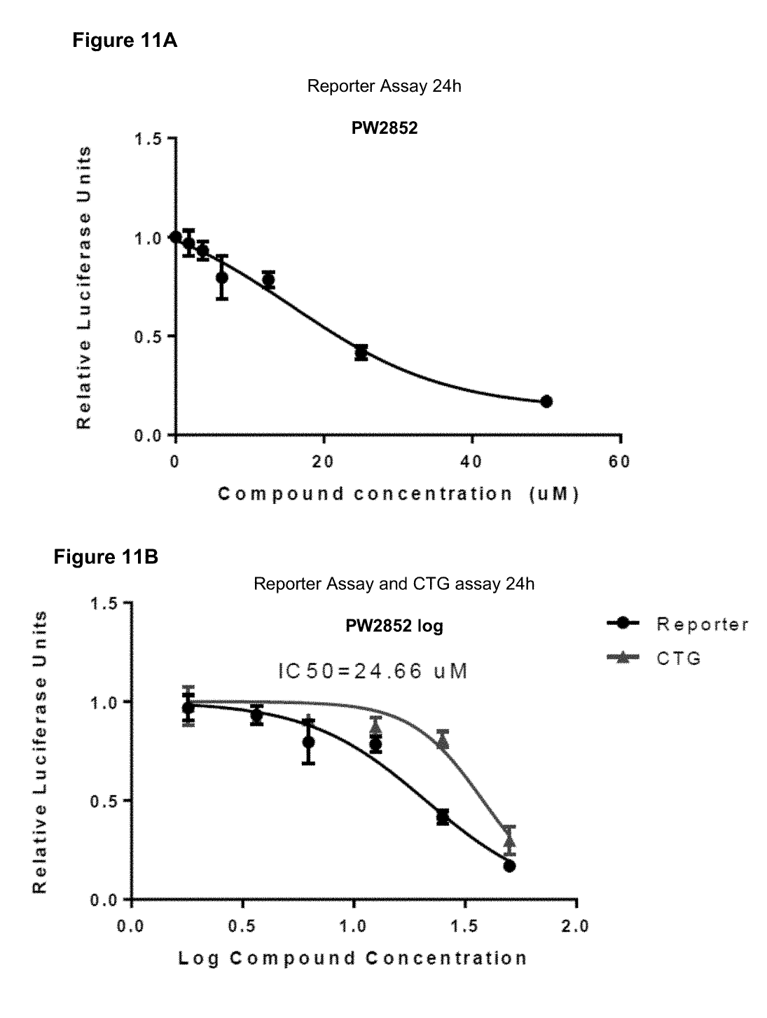

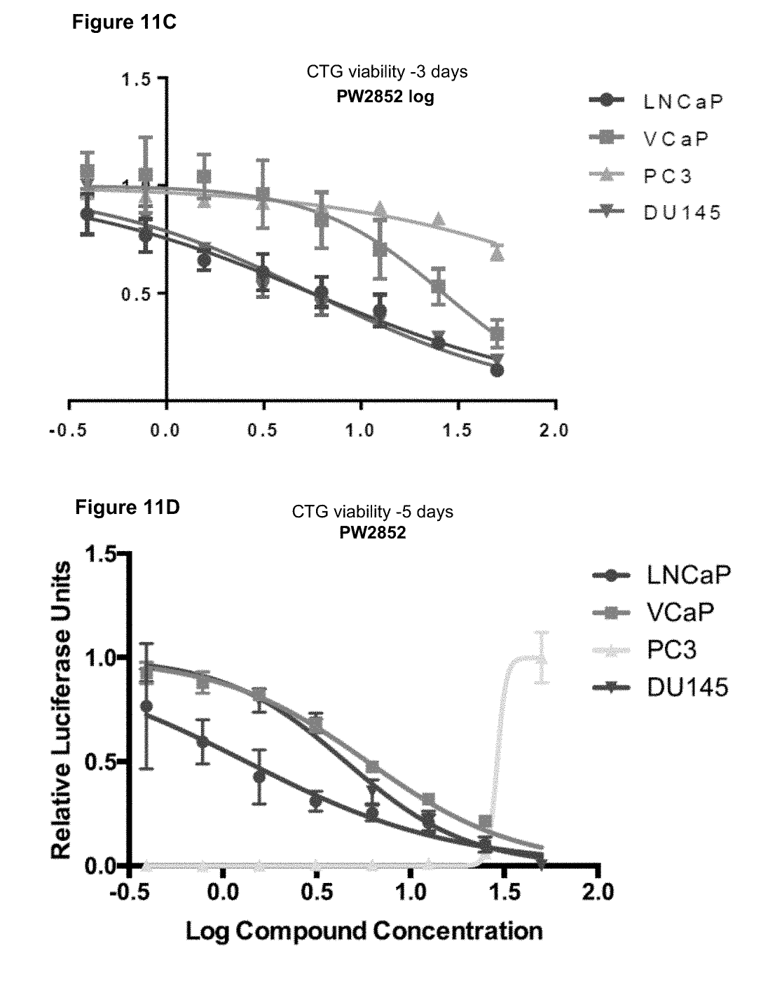

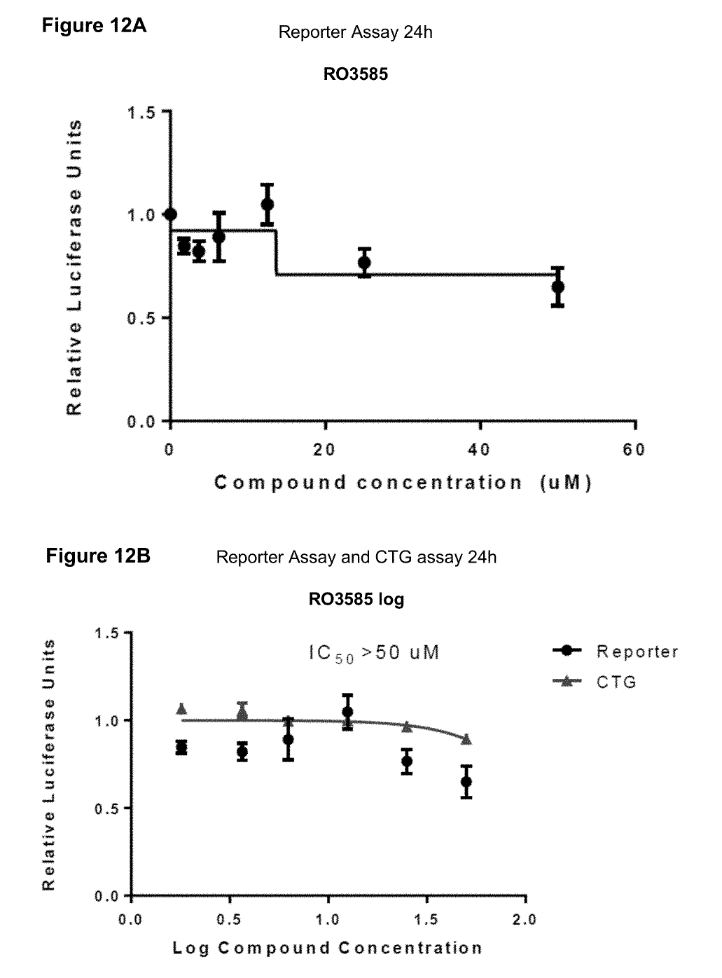

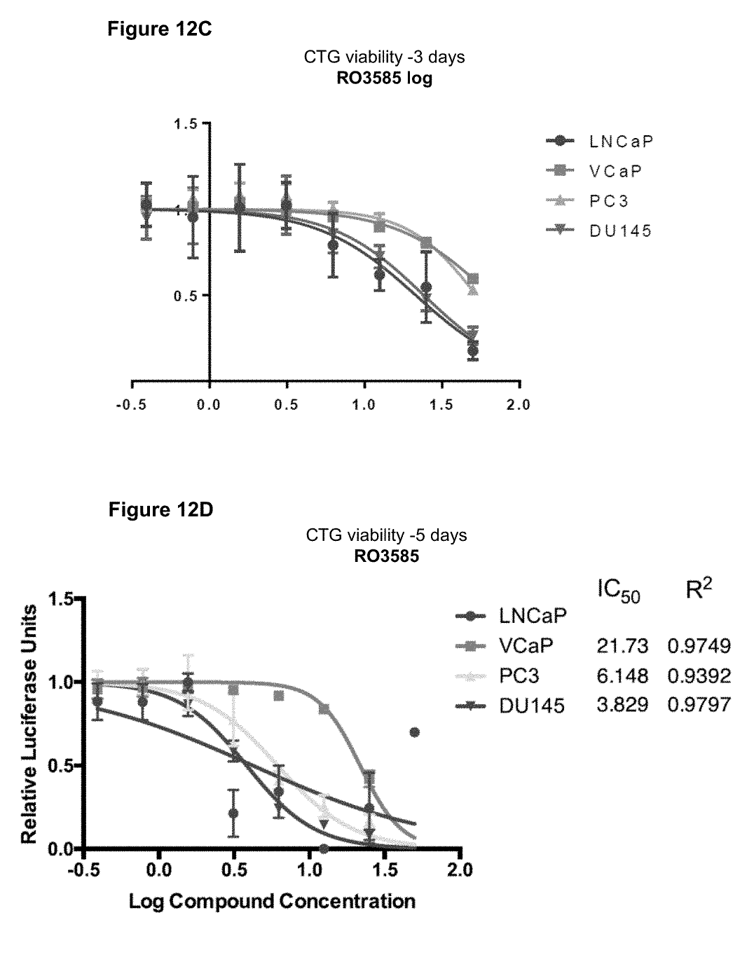

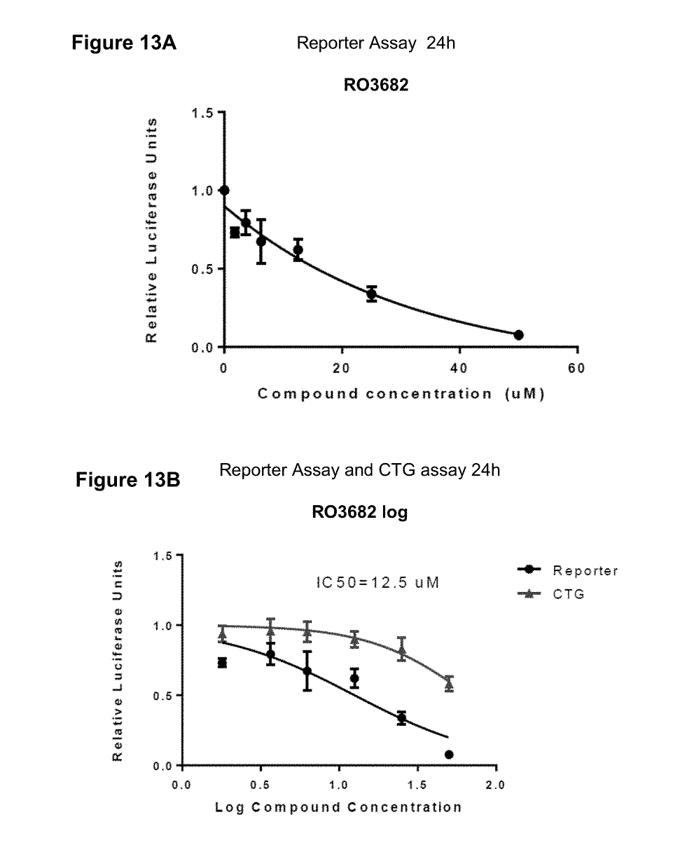

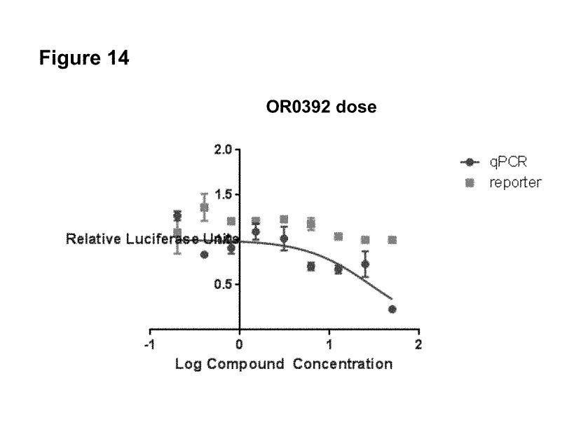

As shown in FIG. 1, a plurality of compounds was used to generate a small molecule microarray (SMM) chip and reveal potential binders of AR-v7. In total, 50,000 molecules were screened against the androgen receptor variant AR-v7 (exons 1-3 truncate of AR-FL) using a small molecule microarray (SMM) (see FIG. 1). 1,378 compounds were selected as preliminary AR-v7 “hits,” passing internal QC measures to eliminate promiscuous and non-specific binders and a cutoff z-score of 1.96 with 95% confidence. As shown in FIG. 2, the selected compounds were evaluated in a qPCR assay. PSA was used as a readout for both AR-v7 and AR activity in the qPCR assay in LNCaP cells. Selected SMM assay positives were administered to cells at two doses (10 and 30 μM) and PSA expression was evaluated using the Single Shot qPCR kit (BioRad) (see FIG. 2). This assay enabled the generation of an initial list of 85 prioritized compounds that demonstrated some potential inhibitory activity. The 85 compounds were further evaluated in a reporter assay using a stable LNCaP cell line with AR-v7 under DOX control and MMTV firefly luciferase (see FIG. 3). The combined analysis of the qPCR assay and reporter assay generated a list of 28 compounds that demonstrated reproducible inhibitory activity. This set was finally evaluated in a dose-dependent manner starting at 50 μM (top concentration serially diluted in 1:1) in qPCR and reporter assay that led to the prioritization of 9 compounds. The top nine compounds were then labeled as follows: KI-ARv-01, KI-ARv-02, KI-ARv-03, KI-ARv-04, KI-ARv-05, KI-ARv-06, KI-ARv-07, KI-ARv-08, and KI-ARv-09. Summaries of the assays of the foregoing nine compounds are shown in FIGS. 4A-14. The most potent compound, labeled as KI-ARv-01 (see FIGS. 4A-4D), demonstrated an initial promising IC₅₀ of 5.43 μM. A second compound, KI-ARv-02 (see FIG. 5), also displayed a relatively similar IC₅₀ of 6.86 μM. As a result of the preliminary assessment, the KI-ARv-01 through KI-ARv-09 compounds provide the foundation for targeting AR-vs in metastatic CRPC.

The presently disclosed compounds KI-ARv-01 through KI-ARv-09 provide the foundation for targeting AR- vs in metastatic CRPC. Commercialization of a therapeutic that targets a resistance mechanism in CRPC is vital to the survival of prostate cancer patients. The nine KI-ARv compound set have the potential to unmask novel AR biology leading to a redesign of therapeutic strategies targeting AR. Further understanding of AR biology could lead to treatments equal to a combined value of current CRPC treatments.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims. Where the claims or description relate to a product (e.g., a composition of matter), it should be understood that methods of making or using the product according to any of the methods disclosed herein, and methods of using the product for any one or more of the purposes disclosed herein, are encompassed by the present disclosure, where applicable, unless otherwise indicated or unless it would be evident to one of ordinary skill in the art that a contradiction or inconsistency would arise. Where the claims or description relate to a method, it should be understood that product(s), e.g., compositions of matter, device(s), or system(s), useful for performing one or more steps of the method are encompassed by the present disclosure, where applicable, unless otherwise indicated or unless it would be evident to one of ordinary skill in the art that a contradiction or inconsistency would arise.

Where ranges are given herein, embodiments are provided in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, embodiments that relate analogously to any intervening value or range defined by any two values in the series are provided, and that the lowest value may be taken as a minimum and the greatest value may be taken as a maximum. Where a phrase such as “at least”, “up to”, “no more than”, or similar phrases, precedes a series of numbers herein, it is to be understood that the phrase applies to each number in the list in various embodiments (it being understood that, depending on the context, 100% of a value, e.g., a value expressed as a percentage, may be an upper limit), unless the context clearly dictates otherwise. For example, “at least 1, 2, or 3” should be understood to mean “at least 1, at least 2, or at least 3” in various embodiments. It will also be understood that any and all reasonable lower limits and upper limits are expressly contemplated where applicable. A reasonable lower or upper limit may be selected or determined by one of ordinary skill in the art based, e.g., on factors such as convenience, cost, time, effort, availability (e.g., of samples, agents, or reagents), statistical considerations, etc. In some embodiments an upper or lower limit differs by a factor of 2, 3, 5, or 10, from a particular value. Numerical values, as used herein, include values expressed as percentages. For each embodiment in which a numerical value is prefaced by “about” or “approximately”, embodiments in which the exact value is recited are provided. For each embodiment in which a numerical value is not prefaced by “about” or “approximately”, embodiments in which the value is prefaced by “about” or “approximately” are provided. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. In some embodiments a method may be performed by an individual or entity. In some embodiments steps of a method may be performed by two or more individuals or entities such that a method is collectively performed. In some embodiments a method may be performed at least in part by requesting or authorizing another individual or entity to perform one, more than one, or all steps of a method. In some embodiments a method comprises requesting two or more entities or individuals to each perform at least one step of a method. In some embodiments performance of two or more steps is coordinated so that a method is collectively performed. Individuals or entities performing different step(s) may or may not interact.

Section headings used herein are not to be construed as limiting in any way. It is expressly contemplated that subject matter presented under any section heading may be applicable to any aspect or embodiment described herein.

Embodiments or aspects herein may be directed to any agent, composition, article, kit, and/or method described herein. It is contemplated that any one or more embodiments or aspects can be freely combined with any one or more other embodiments or aspects whenever appropriate. For example, any combination of two or more agents, compositions, articles, kits, and/or methods that are not mutually inconsistent, is provided. It will be understood that any description or exemplification of a term anywhere herein may be applied wherever such term appears herein (e.g., in any aspect or embodiment in which such term is relevant) unless indicated or clearly evident otherwise.

1. A method of modulating an androgen receptor, or variant thereof, wherein the method comprises exposing the androgen receptor or fragment thereof to a compound selected from the group consisting of: 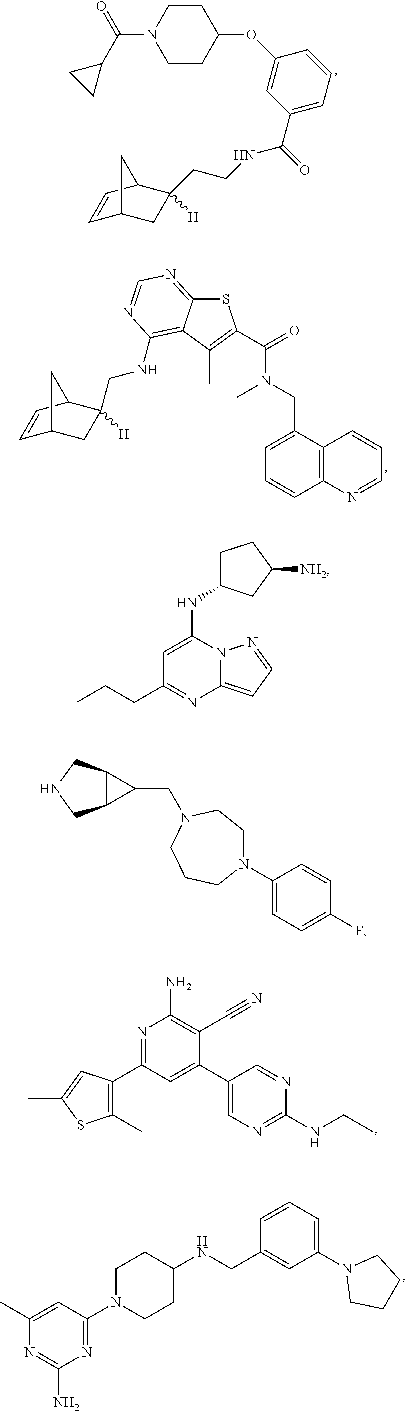 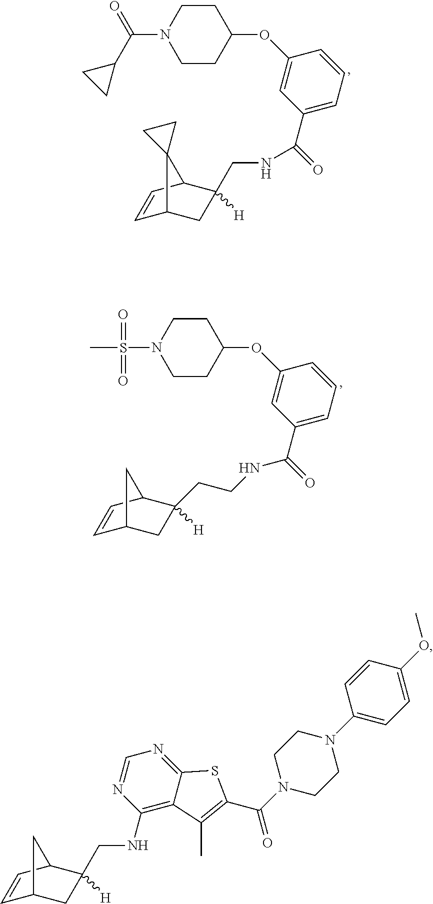 [SMILES: O=C(NCC1CC2C=CC1C21CC1)c1cccc(OC2CCN(C(=O)C3CC3)CC2)c1 ; CS(=O)(=O)N1CCC(Oc2cccc(C(=O)NCCC3CC4C=CC3C4)c2)CC1 ; COc1ccc(N2CCN(C(=O)c3sc4ncnc(NCC5CC6C=CC5C6)c4c3C)CC2)cc1]
and pharmaceutically acceptable salts thereof.

2. (canceled)

3. The method of claim 1, wherein the method comprises exposing a cell expressing the androgen receptor or fragment thereof to a compound selected from the group consisting of: 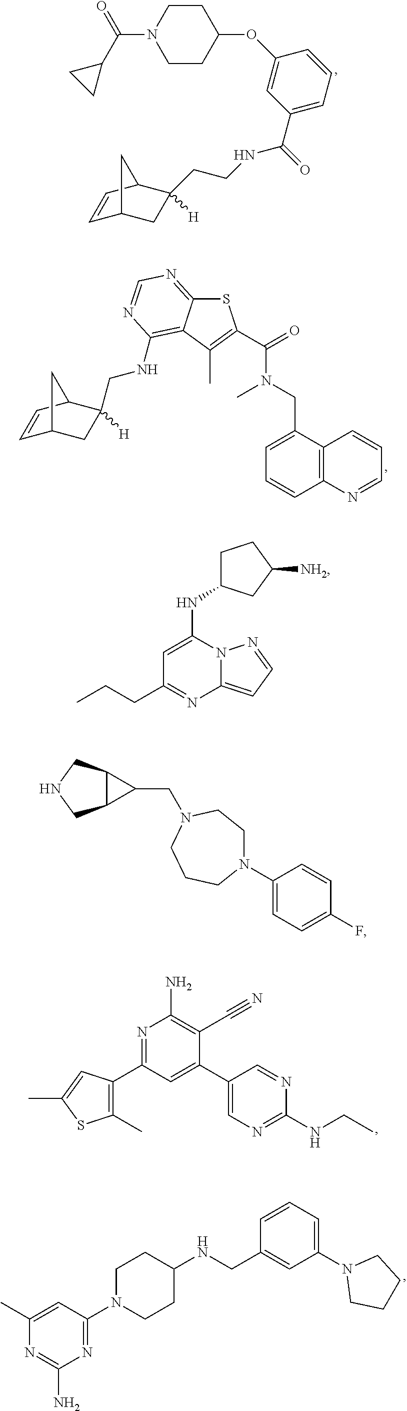 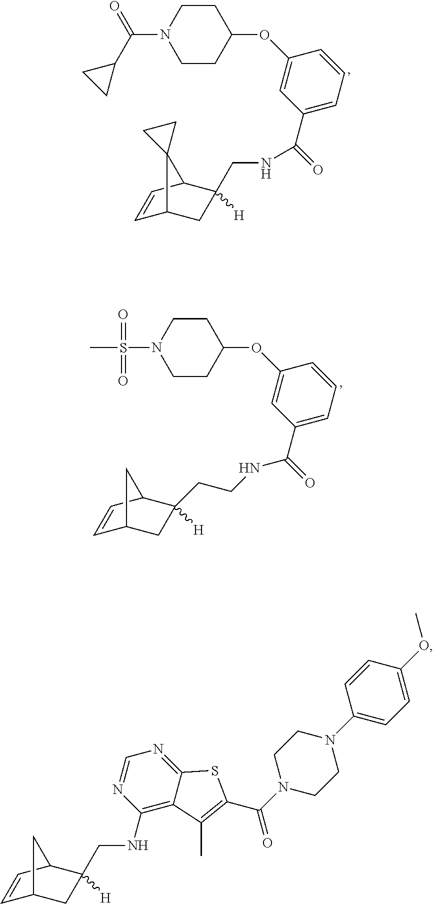 [SMILES: O=C(NCC1CC2C=CC1C21CC1)c1cccc(OC2CCN(C(=O)C3CC3)CC2)c1 ; CS(=O)(=O)N1CCC(Oc2cccc(C(=O)NCCC3CC4C=CC3C4)c2)CC1 ; COc1ccc(N2CCN(C(=O)c3sc4ncnc(NCC5CC6C=CC5C6)c4c3C)CC2)cc1]
and pharmaceutically acceptable salts thereof.

4-5. (canceled)

6. The method of claim 1, wherein the androgen receptor, or variant thereof, is an androgen receptor variant (AR-v).

7. (canceled)

8. The method of claim 6, wherein the AR-v is selected from the group consisting of AR23, ARQ640X, AR-v1, AR-v2, AR-v3, AR-v4, AR-v5, AR-v6, AR-v7, AR-v8, AR-v9, AR-v10, AR-v11, AR-v12, AR-v13, AR-v14, AR-v15, AR-v16, AR-v18, AR8, and AR45.

9. (canceled)

10. A method of modulating the expression of a gene in a cell, comprising exposing a cell expressing an androgen receptor or fragment thereof to an androgen receptor modulator selected from the group consisting of: 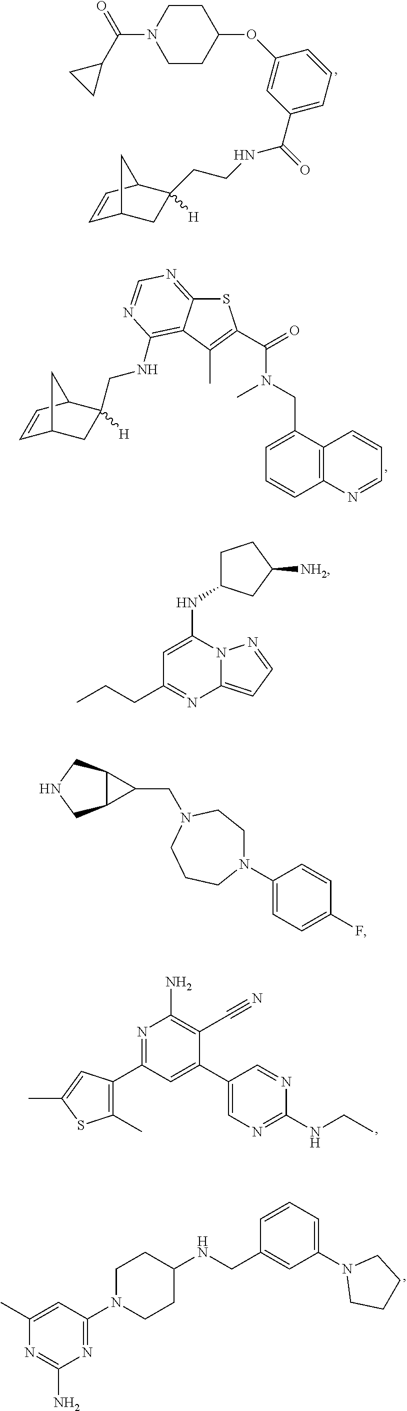 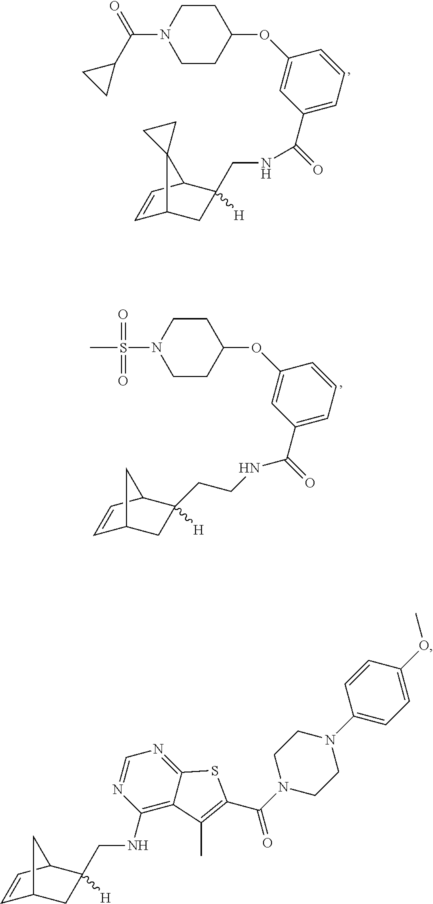 [SMILES: O=C(NCC1CC2C=CC1C21CC1)c1cccc(OC2CCN(C(=O)C3CC3)CC2)c1 ; CS(=O)(=O)N1CCC(Oc2cccc(C(=O)NCCC3CC4C=CC3C4)c2)CC1 ; COc1ccc(N2CCN(C(=O)c3sc4ncnc(NCC5CC6C=CC5C6)c4c3C)CC2)cc1]
and pharmaceutically acceptable salts thereof, wherein the gene is under direct or indirect control of said androgen receptor or fragment thereof.

11. (canceled)

12. The method of claim 10, wherein the gene is AKT1.

13. (canceled)

14. The method of claim 10, wherein the cell is a prostate cancer cell.

15-16. (canceled)

17. A method of treating a disease, wherein the method comprises administering to a subject in need thereof an androgen receptor modulator selected from the group consisting of: 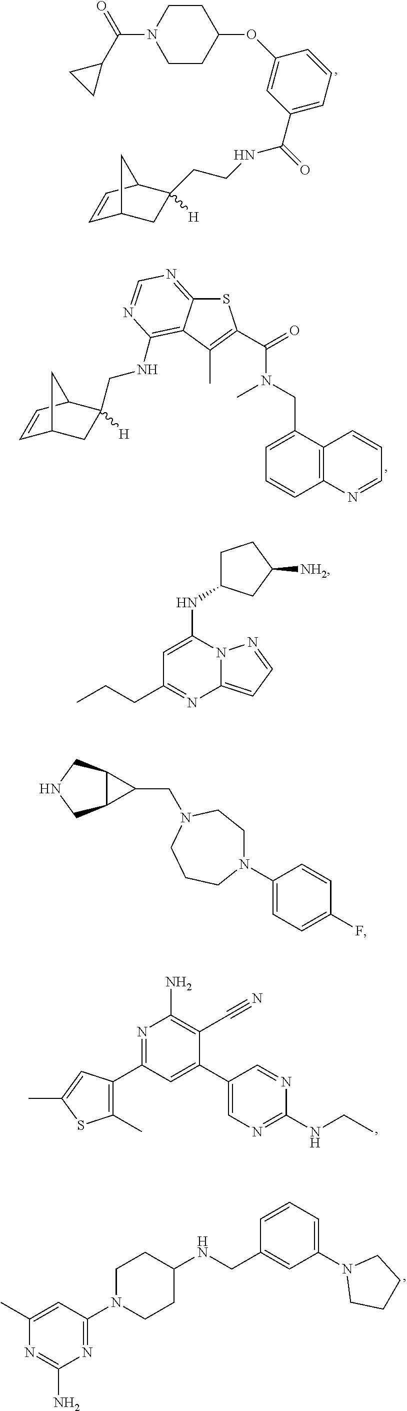 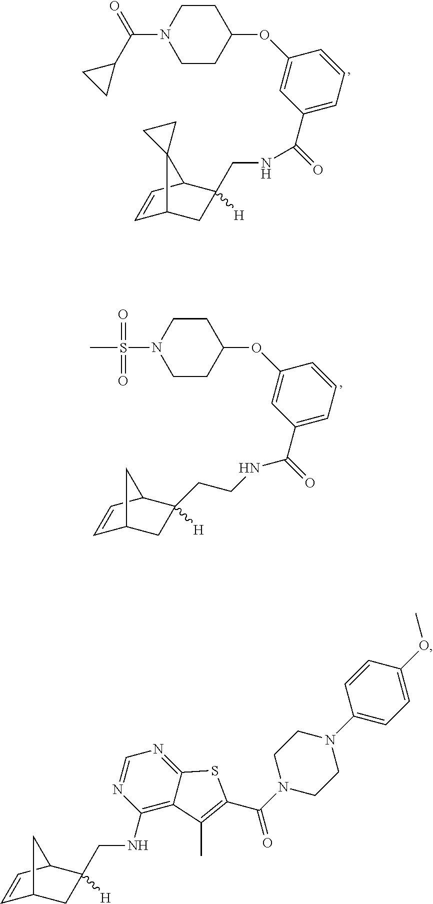 [SMILES: O=C(NCC1CC2C=CC1C21CC1)c1cccc(OC2CCN(C(=O)C3CC3)CC2)c1 ; CS(=O)(=O)N1CCC(Oc2cccc(C(=O)NCCC3CC4C=CC3C4)c2)CC1 ; COc1ccc(N2CCN(C(=O)c3sc4ncnc(NCC5CC6C=CC5C6)c4c3C)CC2)cc1]
and pharmaceutically acceptable salts thereof.

18. The method of claim 17, wherein the disease is a proliferative disease.

19. The method of claim 18, wherein the proliferative disease is cancer.

20. The method of claim 19, wherein the cancer is prostate cancer.

21-22. (canceled)

23. The method of claim 17, further comprising administering an additional therapeutic agent.

24. The method of claim 17, wherein the additional therapeutic agent is a chemotherapeutic.

25-26. (canceled)

27. A pharmaceutical composition comprising a pharmaceutically acceptable excipient and an androgen receptor modulator selected from the group consisting of: 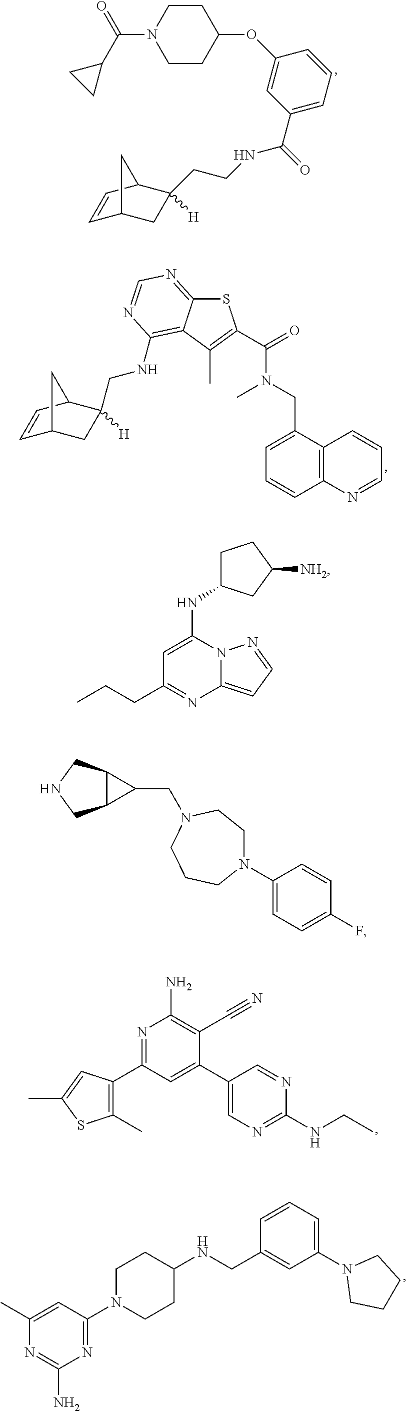 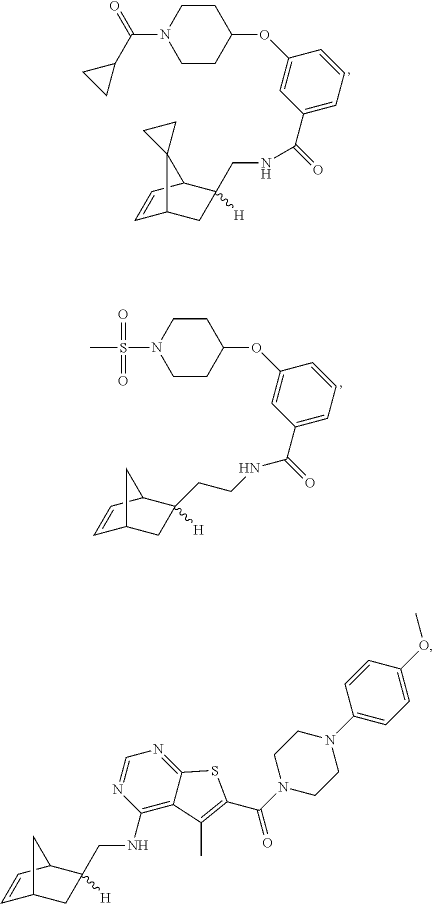 [SMILES: O=C(NCC1CC2C=CC1C21CC1)c1cccc(OC2CCN(C(=O)C3CC3)CC2)c1 ; CS(=O)(=O)N1CCC(Oc2cccc(C(=O)NCCC3CC4C=CC3C4)c2)CC1 ; COc1ccc(N2CCN(C(=O)c3sc4ncnc(NCC5CC6C=CC5C6)c4c3C)CC2)cc1]
and pharmaceutically acceptable salts thereof.

28. A method of treating a disease by administering to a subject the pharmaceutical composition of claim 27.

29-43. (canceled)

44. A method of identifying an androgen receptor modulator comprising:
a. exposing a plurality of compounds to an androgen receptor, or variant thereof;
b. selecting one or more compounds that bind to the androgen receptor, or variant thereof;
c. exposing the one or more selected compounds that bind the androgen receptor, or variant thereof, individually to cells expressing an androgen receptor or fragment thereof;
d. selecting one or more compounds that reduce prostate-specific antigen (PSA) expression in the cells;
e. exposing the one or more compounds selected to reduce PSA expression individually to cells expressing: i) an androgen receptor, or variant thereof, and ii) a reporter protein; and
f. selecting one or more compounds that reduce reporter protein activity level.

45-47. (canceled)

48. The method of claim 44, wherein the step of exposing a plurality of compounds to an androgen receptor or fragment thereof further comprises exposing a small molecule microarray (SMM) comprising a plurality of compounds to the androgen receptor, or variant thereof.

49. The method of claim 44, wherein the step of selecting a compound that reduces PSA expression further comprises:
a. measuring a first PSA expression level and a second PSA expression level with qPCR, wherein the first PSA expression level is the PSA expression level of cells contacted with a selected compound and the second PSA expression level is the PSA expression level of untreated cells;
b. comparing the first PSA expression level and the second PSA expression level; and
c. selecting one or more compounds that reduce the first PSA expression level by at least 10% compared to the second PSA expression level.

50. The method of claim 44, wherein the step of exposing to a compound selected to reduce PSA expression in the cells further comprises:
a. measuring a first reporter protein activity level and a second reporter protein activity level, wherein the first reporter protein activity level is the reporter protein activity level of cells treated with a compound selected for binding to an androgen receptor, or variant thereof, and the second reporter protein activity level is the reporter protein activity level of untreated cells, and wherein the first reporter protein activity level and the second reporter activity level are relative luciferase activity;
b. comparing the first reporter protein activity level and the second reporter protein activity level; and
c. selecting one or more compounds that reduce the first reporter protein activity level by at least 10% compared to the second reporter protein activity level.

51-60. (canceled)

61. A compound identified by the method of claim 44, wherein the compound is a modulator of an androgen receptor, or variant thereof.
